SEQUENCE LISTING (4 sequences)

SEQ ID NO: 1 - protein, 5 aa, Artificial Sequence
  Synthetic construct
KLVFF

SEQ ID NO: 2 - protein, 5 aa, Artificial Sequence
  Synthetic construct
  MISC_FEATURE (1)..(5): All amino acids in this sequence are non-natural enantiomers of the listed amino-acids.
KLVFF

SEQ ID NO: 3 - protein, 5 aa, Artificial Sequence
  Synthetic construct
FFVLK

SEQ ID NO: 4 - protein, 12 aa, Artificial Sequence
  Synthetic construct
YCDGFYACYMDV

(19) United States
(12) Patent Application Publication
(10) Pub. No.: US 2022/0387633 A1
(43) Pub. Date: Dec. 8, 2022

(54) SMART PEPTIDES AND TRANSFORMABLE NANOPARTICLES FOR CANCER IMMUNOTHERAPY

(71) Applicant: The Regents of the University of California, Oakland, CA (US)

(72) Inventors: Kit S. LAM, Davis, CA (US); Lu ZHANG, Sacramento, CA (US)

(21) Appl. No.: 17/634,410

(22) PCT Filed: Aug. 14, 2020
(86) PCT No.: PCT/US20/46495

§ 371 Date: Feb. 10, 2022

Related U.S. Application Data
(60) Provisional application No. 62/886,698, filed on Aug. 14, 2019.
(60) Provisional application No. 62/886,718, filed on Aug. 14, 2019.

(51) Int. Cl. A61K 49/00; A61K 47/64; A61K 47/69; A61K 47/54; A61P 35/00

(52) U.S. Cl. CPC A61K 49/0056; A61K 47/64; A61K 47/6929; A61K 47/546; A61P 35/00; A61K 49/0093; A61K 49/0036

(57) ABSTRACT

The present invention provides a compound of formula (I): A-B-C (I), wherein A is a hydrophobic moiety; B is a peptide, wherein the peptide forms a beta-sheet; and C is a hydrophilic targeting ligand, wherein the hydrophilic targeting ligand is a LLP2A prodrug, LLP2A, LXY30, LXW64, DUPA, folate, a LHRH peptide, a HER2 ligand, an EGFR ligand, or a toll-like receptor agonist CpG oligonucleotides. The present invention also provides nanocarriers comprising compounds of the present invention, nanofibril formation from the nanocarriers, and methods of using the nanocarriers for treating diseases and imaging.

### CROSS-REFERENCES TO RELATED APPLICATIONS

This application is a U.S. National Phase Application of PCT International Application No. PCT/US2020/046495, filed Aug. 14, 2020, which claims priority to U.S. Provisional Application Nos. 62/886,698 and 62/886,718, both filed on Aug. 14, 2019, each of which is incorporated herein in its entirety for all purposes.

### STATEMENT AS TO RIGHTS TO INVENTIONS MADE UNDER FEDERALLY SPONSORED RESEARCH AND DEVELOPMENT

This invention was made with Government support under Grant Nos. R01EB012569 and U01CA198880, awarded by the National Institutes of Health. The Government has certain rights in the invention.

### BACKGROUND OF THE INVENTION

Clinical success in cancer immunotherapy in recent years has brought great enthusiasm to our war against cancer. Immune checkpoint receptor pathway blockade monoclonal antibodies such as anti-PD-1, anti-PD-L1, and anti-CTLA-4 can reverse T effector cell (Teff) dysfunction and exhaustion, resulting in dramatic tumour shrinkage and sometimes complete remission in some patients, even with late stage metastatic diseases. However, the response rate varies greatly between tumour types: up to 40% in melanoma, 25% in non-small cell lung cancer, but <10% in most other tumour types. To date, US Food and Drug Administration (FDA) had approved seven immune checkpoint blockade monoclonal antibodies (ICB-Ab): one CTLA-4 inhibitor (ipilimumab), three PD-1 inhibitors (nivolumab, pembrolizumab, and cemiplimab), and three PD-L1 inhibitors (atezolizumab, durvalumab, and avelumab), used either alone, or in combination with other chemotherapies, against a range of tumour types.

Tumour microenvironment (TME), comprised of immune and stromal cells, vasculature, extracellular matrix, cytokines, chemokines, and growth factors, can all influence tumour response to immune checkpoint blockage (ICB) therapies. Emerging data indicates that defects in Teff cell homing to the tumour sites is a critical factor in resistance to ICB therapy. Other mechanisms of ICB resistance include the presence of immunosuppressive regulatory T cells (Tregs), myeloid-derived suppressor cells (MDSCs), and M2 macrophages at the tumour sites. Elevated level of CCL5, CCL17, CCL22, CXCL8 and CXCL12 facilitates the recruitment of Tregs and MDSCs to the TME, resulting in a diminished ICB response. In contrast, CXCL9 and CXCL10 promote homing of cytotoxic T-cells (CTLs) to the tumour sites, boosting anti-tumour immune response; transforming growth factor beta (TGF-β) does the opposite and also upregulates Tregs. VEGF upregulates inhibitory receptors on CTLs, contributing to their exhaustion. Upregulation of other immune checkpoint receptors such as mucin domain-3 protein (TIM-3), lymphocyte-activation gene 3 (LAG-3), B and T lymphocyte attenuator (BTLA), T-cell immunoreceptor, tyrosine-based inhibition motif domain (TIGIT), and V-domain immunoglobulin-containing suppressor of T-cell activation (VISTA) has been implicated in ICB resistance. Co-expression of these checkpoint receptors can lead to T cell exhaustion. Oncogenic or tumour suppressor pathways, such as mitogen-activated protein kinase (MAPK) and PI3K-γ in the cancer cells can also influence TME by altering the immune cell compositions and cytokine profile, contributing to ICB resistance. Inhibitors against these pathways have been found to improve ICB response.

In an attempt to overcome ICB resistance, many combination therapeutic strategies have been tried preclinically and clinically. These include the addition of the following drugs to a ICB-Ab: one other ICB-Ab (antibodies against CTLA-4, PD-1, PD-L1, LAG-3 and TIM-3), chemotherapeutic agents (paclitaxel, gemcitabine and carboplatin), radiation therapy, targeted therapy (inhibitors against PI3K, VEGF, BRAF/MEK, IDO, A2AR, FGFR, EGFR, PARP and mTOR), macrophage inhibitors (inhibitors against CSF1R and ARG1), cytokine/chemokine inhibitors (inhibitors against CXCR4, CXCR2 and TGF-β), epigenetic modulators (histone deacetylase inhibitors and hypomethylating agents), immunomodulatory agents (antibodies against OX40, 41BB, GITR, CD40 and ICOS), adoptive cell transfer therapy (car T, TIL and TCR), and modulation of gut microbiome.

Advancement and optimization of nano-immunotherapy lie in the development of innovative approaches to enhance the specificity and controllability of immunotherapeutic interventions, targeting desired cell types at the TME. Advanced bionanomaterials or approaches in a more controlled manner could enhance immunotherapeutic potency by increasing the accumulation and prolonging the retention of immunomodulatory and immune cell homing agents at the TME while sparing the normal tissues and organs, thus reducing off-target adverse effects such as systemic cytokine storm. In situ assembly of nanomaterial has been demonstrated to improve the performance of bioactive molecules. One plausible explanation is that T cell targeting ligands and/or immunomodulatory agents incorporated into in situ fibrillar-transformable nanoplatform, will generate nanofibrillar networks at the TME, enhancing Teff cells homing to the tumour sites and improving immunotherapeutic efficacy, with or without additional ICB therapy.

Human epidermal growth factor receptor 2 (HER2) is overexpressed in over 20% breast cancers, and to a lesser degree in gastric cancers, colorectal cancer, ovarian cancers and bladder cancers. Unlike those cancers caused by mutated or fusion oncogenes (e.g. EGFR in lung cancers and Bcr-Abl in chronic myelocytic leukemia) which respond well to monotherapy, cancers with HER2 overexpression often require drug combinations. It is because this latter group of tumours are driven by gene amplification and massive overexpression of HER2. HER2 is a receptor tyrosine kinase that is normally activated via induced dimerization with itself or with its family members EGFR, HER3 or HER4. In HER2 positive tumours, HER2s are massively overexpressed and constitutively dimerized, leading to unrelenting activation of down-stream proliferation and survival pathways and malignant phenotype.

Because of the high expression level of HER2, trastuzumab and pertuzumab, the two anti-HER2 monoclonal antibodies are ineffective as monotherapy against these tumours. They need to be given in combinations with other HER2-targeted therapy, chemotherapy or hormonal therapy. Herein, some embodiments describe a novel HER2-mediated, peptide-based, and non-toxic transformative nano-agent that is highly efficacious as a monotherapy against HER2+ breast cancer xenograft models. This receptor-mediated transformable nanotherapy (RMTN) is comprised of peptide with unique domains that allow self-assembly to form micelles under aqueous condition and transformation into nanofibrils at the tumour site, where HER2 is encountered. The resulting nanofibrillar network effectively suppresses HER2 dimerization and downstream signaling, and facilitates tumour cell death.

Herein, smart supramolecular materials for cancer immunotherapy were constructed.

### BRIEF SUMMARY OF THE INVENTION

In one embodiment, the present invention provides a compound of formula (I): A-B-C (I), wherein A is a hydrophobic moiety; B is a peptide, wherein the peptide forms a beta-sheet; and C is a hydrophilic targeting ligand, wherein the hydrophilic targeting ligand is a LLP2A prodrug, LLP2A, LXY30, LXW64, DUPA, folate, a LHRH peptide, a HER2 ligand, an EGFR ligand, or a toll-like receptor agonist CpG oligonucleotides.

In another embodiment, the present invention provides a compound of formula (I): A-B-C (I), wherein A is a hydrophobic moiety; B is a peptide, wherein the peptide forms a beta-sheet; and C is a hydrophilic targeting ligand, wherein the hydrophilic targeting ligand is a LLP2A prodrug, LLP2A, LXY30, LXW64, DUPA, a LHRH peptide, a HER2 ligand, an EGFR ligand, or a toll-like receptor agonist CpG oligonucleotides and wherein when the hydrophobic moiety is bis-pyrene, then C is a LLP2A prodrug, LLP2A, LXY30, LXW64, DUPA, a LHRH peptide, an EGFR ligand, or a toll-like receptor agonist CpG oligonucleotides.

In another embodiment, the present invention provides a nanocarrier having an interior and an exterior, the nanocarrier comprising a plurality of compounds of the present invention, wherein each compound self-assembles in an aqueous solvent to form the nanocarrier such that a hydrophobic pocket is formed in the interior of the nanocarrier, and a hydrophilic group self-assembles on the exterior of the nanocarrier.

In another embodiment, the present invention provides a nanocarrier having an interior and an exterior, the nanocarrier comprising a plurality of a first conjugate and a second conjugate wherein the first conjugate comprises formula (I): A-B-C (I); and the second conjugate comprises formula (II): A′-B′-C′ (II) wherein: A and A′ are each independently a hydrophobic moiety; B and B′ are each independently a peptide, wherein each peptide independently forms a beta-sheet; and C and C′ are each independently a hydrophilic targeting ligands, wherein each hydrophilic targeting ligand is independently a LLP2A prodrug, LLP2A, LXY30, LXW64, DUPA, folate, a LHRH peptide, a HER2 ligand, an EGFR ligand, or a radiometal chelator; and wherein A and A′ are different hydrophobic moieties and/or C and C′ are different hydrophilic targeting ligands.

In another embodiment, the present invention provides a method of forming nanofibrils, comprising contacting a nanocarrier of the present invention with a cell surface or acellular component at a tumor microenvironment, wherein the nanocarrier undergoes in situ transformation to form fibrillary structures, thereby forming the nanofibrils.

In another embodiment, the present invention provides a method of treating a disease, comprising administering to a subject in need thereof, a therapeutically effective amount of a nanocarrier of the present invention, wherein the nanocarrier forms nanofibrils in situ after binding to a cell surface or acellular component at the tumor microenvironment, thereby treating the disease.

in another embodiment, the present invention provides a method of imaging, comprising administering to a subject to be imaged, an effective amount of a nanocarrier of the present invention.

### BRIEF DESCRIPTION OF THE DRAWINGS

FIGS. 1A-1F show assembly and fibrillar-transformation of transformable peptide monomer 1 (TPM1′) BP-FFVLK-YCDGFYACYMDV. FIGS. 1A-1B show changes in UV-vis absorption (FIG. 1A) and fluorescence (FIG. 1B) of NPs1 upon gradual addition of water into DMSO solution of NPs1 with the H2O:DMSO from 0:100, 20:80, 40:60, 60:40, 80:20, 90:10, 98:2 and 99.5:0.5. Ex=380 nm. FIG. 1C TEM images of initial NPs1 and nanofibers (NFs1) transformed by NPs1 interaction with HER2 protein (Mw≈72 KDa) at different time points (0.5, 6, 24 h). The scale bar in d: 100 nm. FIGS. 1D-1F—show variation of size distribution (FIG. 1D), CD spectra (FIG. 1E) and fluorescence signal (FIG. 1F) of initial NPs1 and NFs1 at the different time points. The molar ratio of HER2 peptide/HER2 protein was approximately 1000:1.

FIGS. 2A-2H show the morphological characterizations of fibrillar-transformable NPs1 co-culture with HER2 positive cancer cells. FIGS. 2A-2C show cellular fluorescence distribution images of NPs1 interaction with SKBR-3 cells (HER2+) (FIG. 2A), BT474 cells (HER2+) (FIG. 2B) and MCF-7 cells (HER2−) (FIG. 2C) at 6 h time point. Scale bar in FIGS. 2A-2C: 50 μm. FIG. 2D shows Western blot and quantitative analysis of relative HER2 protein expression in MCF-7 cells and MCF-7/C6 cells. ***P<0.001. FIG. 2E shows cellular fluorescence distribution images of NPs1 interaction with MCF-7/C6 cells (HER2+) at the different time points (0.5, 6, 24 h). Scale bar in e: 50 μm. FIG. 2F shows fluorescence binding distribution images of the nanofibrillar network of NFs1 and HER2 antibody (29D8, rabbit, different receptor binding site with HER2 peptide of NPs1) on the cell membrane of MCF-7/C6 cells. HER2 antibody was used to label HER2 receptors. FIG. 2G shows SEM images of untreated MCF-7/C6 cells and cells treated by NPs1 for 6 h and 24 h. FIG. 2H shows TEM images of untreated MCF-7/C6 cells and cells treated by NPs1 for 24 h. The red arrow shows fibrillar network. The concentration of NPs1 was 50 μM.

FIGS. 3A-3G show the extracellular and intracellular mechanisms of fibrillar-transformable NPs interaction with MCF-7/C6 breast cancer cells. FIG. 3A shows cellular fluorescence distribution images of NPs1, NPs2 and HER2 antibody (29D8, rabbit, different receptor binding site with HER2 peptide of NPs1 and NPs2) binding HER2 receptors of MCF-7/C6 cells, respectively. HER2 antibody was used to label HER2 receptors. The concentration of NPs1 and NPs2 were 50 μM. The scale bar in a: 20 μm. FIG. 3B shows the viability of MCF-7/C6 cells incubated with NPs1-4 at the different concentration (n=3). *P<0.05, **P<0.01. FIG. 3C shows Western blot analysis of apoptosis related proteins and HER2 total protein in MCF-7/C6 cells treated by NPs1 for 24 h with different concentration. FIGS. 3D-3E show Western blot analysis of inhibition and disaggregation mechanism of HER2 protein dimer in MCF-7/C6 cells treated by NPs1 for 24 h with different concentration (FIG. 3D) and at 50 μM under different time point (FIG. 3E). FIG. 3F shows Western blot analysis of inhibition mechanism of proliferation protein in MCF-7/C6 cells treated by NPs1 at 50 μM under different time point and at 24 h under different concentration. FIG. 3G shows Western blot analysis of inhibition mechanism of proliferation protein in MCF-7/C6 cells treated by NPs1-4 and Herceptin (HP) at 36 h. The concentration of NPs1-4 were 50 μM, and the concentration of Herceptin was 15 μg/mL as a positive control group.

FIG. 4A-4F show in vivo evaluation of fibrillar-transformable NPs. FIG. 4A show time-dependent ex vivo fluorescence images and FIG. 4B show quantitative analysis of tumour tissues and major organs (heart, liver, spleen, lung, kidney, intestine, muscle and skin) collected at 10, 24, 48, 72 and 168 h post-injection of NPs1. In FIG. 4B ***P<0.001, the fluorescence signal in tumour tissue at 72 h and 168 h compared with other organs displays tumour accumulation and in situ transformation of fibrillar network with long retention time; ***P<0.001, the fluorescence signal in liver and kidney at 10 h compared with that at 72 and 168 h displays that NPs1 could be removed rapidly from liver and kidney. FIG. 4C shows the fluorescence distribution images and H&E image of NPs1 in tumour tissue and normal skin tissue at 72 h post-injection (green color: BP of NPs1; blue color: DAPI; scale bar in c: 100 μm). FIG. 4D shows time-dependent ex vivo fluorescence images of tumour tissues and major organs collected at 72 h post-injection of NPs2-4. FIG. 4E shows quantitative analysis of tumour tissues and livers collected at 72 h post-injection of NPs1-4. In FIG. 4E ***P<0.001, the fluorescence signal of tumour tissue in NPs1 group compared with that in other control groups displays that fibrillar networks in NPs1 group promote long retention time in tumour site. FIG. 4F shows TEM images of distribution in tumour tissue and in situ fibrillar transformation of NPs1-4 at 72 h post-i.v. injection and untreated group. The dose of NPs1-4 were 8 mg/Kg per injection. In FIG. 4F, “C” means MCF-7/C6 cell; “N” means cell nucleus.

FIG. 5A-5K show anti-tumour activity of NPs in Balb/c nude mice bearing HER2 positive breast tumour. FIG. 5A shows schematic illustration of tumour inoculation and treatment protocol of mice. FIGS. 5B-5C show observation of the tumour inhibition effect (FIG. 5B) and weight change of mice (FIG. 5C) in subcutaneous tumour model during the 40 days of treatment (n=8 per group; the dose of NPs1-4 were 8 mg/Kg per injection). **P<0.01, ***P<0.001. FIG. 5D shows cumulative survival of different treatment groups of mice bearing MCF-7/C6 breast tumours. FIG. 5E shows schematic illustration of three times treatment protocol of mice for tumour tissue analysis. FIG. 5F shows the fluorescence distribution images in tumour tissue and H&E anti-tumour image post three times injection of NPs1 (green color: BP of NPs1; blue color: DAPI; scale bar in f: 100 μm). FIG. 5G shows representative TEM images of late membrane rapture and cell death by the nanofibrillar network after injection of NPs1 three times. The red arrow shows fibrillar network. FIG. 5H shows Ki-67 stain images of tumour tissues treated by different groups after injection three times. Scale bar in h: 25 μm. FIG. 5I shows Western blot analysis of inhibition mechanism of HER2 protein and proliferation proteins in MCF-7/C6 tumour tissues treated by different groups after injection three times. FIGS. 5J-5K shows observation of the tumour inhibition effect in subcutaneous tumour SKBR-3 (FIG. 5J) and BT474 HER2 positive breast cancer (FIG. 5K) models during the 40 days of treatment (n=8 per group; the dose of NPs1 were 8 mg/Kg per injection). ***P<0.001 compared with PBS control group.

FIG. 6 shows chemical structure and mass spectra via MALDI-TOF of transformable peptide monomer 1 BP-FFVLK-YCDGFYACYMDV.

FIG. 7 shows chemical structure and mass spectra via MALDI-TOF of transformable peptide monomer 2 BP-GGAAK-YCDGFYACYMDV.

FIG. 8 shows chemical structure and mass spectra via MALDI-TOF of transformable peptide monomer 3 BP-FFVLK-PEG.

FIG. 9 shows chemical structure and mass spectra via MALDI-TOF of transformable peptide monomer 4 BP-GGAAK-PEG.

FIG. 10 shows effect of HER2 protein/peptide ligand ratio on fibrillar transformation. TEM images and particle size measurements of NPs1 were obtained after incubation with soluble HER2 protein for 24 h in PBS solution. NPs1 concentration was maintained constant at 20 μM. The scale bar is 200 nm. The HER2 protein/peptide ligand ratio is labeled for each micrograph. Experiments were repeated three times.

FIG. 11A shows observation on the anti-tumour effect in subcutaneous SKBR-3 tumour during the 40 days of treatment (n=6 per group; the dose of NPs1-4 were 8 mg/kg per injection, q.o.d.; data are presented as the mean±s.d.). The statistical significance was calculated via one-way ANOVA with a Tukey post-hoc test. *P<0.05. FIGS. 11B-11C show body weight of mice bearing subcutaneous BT474 tumour (FIG. 11B) and SKBR-3 tumour (FIG. 11C) during the 40 days of treatment (n=6 per group; data are presented as the mean s.d.). Red arrows depict each single i.v. injection.

FIG. 12 shows nanofibrillar networks promote T cell homing and reprogram tumour microenvironment for enhanced immunotherapy. Schematic illustration of self-assembly and fibrillar transformation of TPMs, and (I), (II), (III) process in tumour tissue: in situ fibrillar transformation of NPs, LLP2A conversion from proLLP2A, followed attracting and targeting T cells, and TAMs re-education of from M2 to M1 phenotype. TPMs, NPs, NFs, M1-TAM and M2-TAM represent transformable peptide monomers, nanoparticles, nanofibrils, M1-like tumour-associated microphage and M2-like tumour-associated microphage, respectively.

FIG. 13A-13H shows assembly and fibrillar transformation of transformable peptide TPM1 (LXY30-KLVFFK(Pa)) and TPM2 (proLLP2A-KLVFFK(R848)). FIG. 13A shows schematic illustration of molecular structure and function of TPM1 and TPM2. FIG. 13B shows changes in fluorescence (FL) of T-NPs following the gradual addition of water (from 0 to 99%) to a solution of T-NPs in DMSO comprised of TPM1 and TPM2 at a 1:1 ratio; excitation wavelength, 405 nm. FIG. 13C shows TEM images of initial T-NPs and T-NPs transformed into nanofibrils (T-NFs) after interaction with soluble α₃β₁ integrin protein for 24 h (H₂O to DMSO ratio of 99:1). The concentration of T-NPs used in the experiment was 20 μM. The scale bars in c are 100 nm. FIG. 13D shows variation in fluorescence signal of Pa in the fibrillar-transformation process of T-NPs to T-NFs over time. FIG. 13A E shows TEM images of initial T-NPs and T-NFs after interaction with esterase, soluble α₄β₁ integrin protein or α₄β₁ integrin protein plus esterase for 24 h (H₂O to DMSO ratio of 99:1). The concentration of T-NPs used in the experiment was 20 μM. The scale bars in e are 100 nm. FIGS. 13F-3G show variation in size distribution (FIG. 13F) and circular dichroism spectra (FIG. 13G) of initial T-NPs and T-NFs under different conditions. FIG. 13H shows Tte in vitro release profile of R848 from T-NFs over time. The molar ratio of α₃β₁ or α₄β₁ integrin protein to peptide ligand was approximately 1:1000. a.u., arbitrary units; mdeg, millidegrees.

FIG. 14 shows DLS experiment to confirm transformation of T-NPs to T-NFs. The peak at 20 nm gradually went down in the solution, while the peak around 700 nm went up.

FIG. 15A-15H shows morphological characterization of fibrillar-transformable nanoparticles after incubation with 4T1 murine breast cancer cells. FIG. 15A shows cellular fluorescence distribution images of T-NPs and UT-NPs interaction for 6 h with 4T1 cells. Scale bar is 10 μm. Experiments were repeated three times. FIG. 15B shows cellular fluorescence signal retention images of 4T1 cells after exposure to T-NPs and UT-NPs for 6 h followed by incubation with fresh medium without NPs for 18 h. Scale bar is 10 μm. Experiments were repeated three times. FIG. 15C shows representative TEM images of 4T1 cells treated with T-NPs and UT-NPs for 24 h, showing abundance of nanofibrils around cells treated with T-NPs. Scale bar is 200 nm. Experiments were repeated three times. The concentration of T-NPs was 50 μM. FIG. 15D shows cellular fluorescence distribution images of Jurkat T-lymphoma cells (GFP labeled) after incubation with esterase-treated T-NPs. Jurkat cells were used to mimic T-lymphocytes, which also express α₄β₁ integrin. Scale bar is 10 μm. Experiments were repeated three times. FIG. 15E shows representative SEM images of untreated 4T1 and Jurkat cells, and cells treated with T-NPs for 6 h. Scale bar is 10 μm. Experiments were repeated three times. FIG. 15F shows experimental scheme and cellular fluorescence distribution images of T-NPs (fluorescent red), after interaction with 4T1 and GFP-labeled Jurkat cells. It shows nanofibrillar networks covering 4T1 cells, which in turn could attract and bind Jurkat malignant T-cells. Scale bar is 10 μm. Experiments were repeated three times. FIG. 5G shows representative SEM images of 4T1 and Jurkat cells after treatment with T-NPs (see FIG. 15F). Experiments were repeated three times. FIG. 15H shows representative images of M2-like murine macrophages and subsequent re-education by T-NFs, T-NFs plus esterase, or R848 at different time points. Scale bar is 20 μm. Experiments were repeated three times. Statistical significance was calculated using a two-sided unpaired t test; *P<0.05, **P<0.01, ***P<0.001.

FIG. 16A-16M shows in vivo evaluation of fibrillar-transformable nanoparticles. FIG. 16A-16B show time-dependent ex vivo fluorescence (FL) images (FIG. 16A) and quantitative analysis (FIG. 16B) of tumour tissues and major organs (heart (H), liver (Li), spleen (Sp), lung (Lu), kidney (K), intestine (I), muscle (M) and skin (Sk)) collected at 10, 24, 48, 72, 120 and 168 h post-injection of T-NPs. Data are presented as mean±s.d., n=3 independent experiments. FIG. 16C shows time-dependent ex vivo fluorescence (FL) images of tumour tissues collected at 10, 24, 48, 72, 120 and 168 h post-injection of UT-NPs. Data are presented as mean±s.d., n=3 independent experiments. FIG. 16D shows fluorescence (FL) quantification of tumour tissues collected at 10, 24, 48, 72, 120 and 168 h post-injection of T-NPs and UT-NPs. FIG. 16E shows representative TEM images of distribution in tumour tissue and in situ fibrillar transformation of T-NPs, UT-NPs and untreated control group at 72 h post-injection. “N” depicts nucleus. FIG. 16F shows fluorescence (FL) distribution images of T-NPs in tumour tissue and normal skin tissue at 72 h post-injection (red, Pa of T-NPs; blue, DAPI; scale bars, 50 μm). FIG. 16G shows R484 distribution retention in tumour tissues at different time points post injection of T-NPs and UT-NPs. Dose of R848: 0.94 mg kg⁻¹; data were mean±s.d., n=3 for each time point. FIG. 16H shows the expression of CXCL10 chemokine within the tumour tissues after 3 days of T-NPs, UT-NPs and saline treatment (n=3; data were mean±s.d.). FIG. 16I-16K show representative flow cytometric analysis images of CD45⁺CD3⁺ (FIG. 16I), CD8⁺/CD4⁺ (FIG. 16J) and CD4⁺Foxp3⁺ (FIG. 16K) T cell within the 4T1 tumours excised from mice treated with T-NPs, UT-NPs or saline control. FIG. 16L shows immunohistochemistry (IHC) of tumours excised from mice after treatment with T-NPs or UT-NPs. Representative images are shown for the IHC staining of T cells (CD8⁺, CD4⁺, Foxp3⁺) and macrophage markers (CD68, CD163). Scale bar is 100 μm. FIG. 16M shows the expression levels (qPCR assay) of IFN-γ, TGF-β, IL12, IL10, Nos2 and Arg-1 in 4T1 tumours excised from mice 15 days after treatment with T-NPs or UT-NPs (n=3; data were mean±s.d.). Statistical significance was calculated using a two-sided unpaired t test; *P<0.05, **P<0.01, ***P<0.001.

FIG. 17A-17G shows anti-tumour efficacy of fibrillar-transformable nanoparticles in Balb/c mice bearing 4T1 breast tumour. FIG. 17A shows experimental design: orthotopic tumour inoculation and treatment protocol; regimen 6 is T-NPs with all the 4 critical components. FIG. 17B-17C show Oservation of tumour inhibitory effect (FIG. 17B) and weight change (FIG. 17C) of mice bearing orthotopic 4T1 tumour over 21 d after initiation of treatment (n=8 per group). Data are presented as mean±s.d. FIG. 17D shows cumulative survival of different treatment groups of mice bearing 4T1 breast tumours. FIG. 17E shows representative flow cytometric analysis images of CD3⁺CD8⁺ T cell within the 4T1 tumours excised from treated mice on day 21. FIG. 17F shows H&E and IHC images of excised tumors. Representative images are shown for the IHC staining of Ki67, T cells (CD8, Foxp3) and macrophage markers (CD68, CD163). Scale bar is 100 μm. FIG. 17G shows the expression levels (analyzed by qPCR) of IFN-γ, TNF-α, IL12, IL6, TGF-β, IL10, Nos2 and Arg-1 in 4T1 tumours excised from mice on day 21 (data were mean±s.d.). Statistical significance was calculated using a two-sided unpaired t test; *P<0.05, **P<0.01, ***P<0.001.

FIG. 18A-18L shows anti-tumour efficacy of fibrillar-transformable nanoparticles plus anti-PD-1 therapy in mice bearing 4T1 breast tumour or Lewis lung tumour. FIG. 18A shows experimental design: orthotopic tumour inoculation and treatment protocol (4 treatment arms; regimen 4, 5 and 6 are the same as those shown in FIG. 4a). FIG. 18B shows tumor response in mice bearing orthotopic 4T1 tumour over 21 d of treatment (n=8 per group). Data are presented as mean±s.d. ***P<0.001. FIG. 18C shows cumulative survival of the four treatment groups. FIG. 18D shows experimental design: Mice previously treated with T-NPs (regimen 6) plus anti-PD-1 Ab were rechallenged with re-inoculation of cancer cells on day 90, followed by three q.o.d i.p. doses of anti-PD-1 Ab. FIG. 18E shows no anti-tumor immune memory effect was observed in same age naïve mice. FIG. 18F shows anti-tumor immune memory effect was observed in mice previously treated with T-NPs and anti-PD-1 Ab. FIG. 18G shows cumulative survival of naïve mice and previously T-NPs plus anti-PD-1 treated mice. FIG. 18H-18I show IFN-γ (FIG. 18H) and TNF-α (FIG. 18I) level in mouse sera 6 days after mice were rechallenged with 4T1 tumor cells and a day after the last dose of anti-PD-1 Ab. FIG. 18J-18K shows Observation of tumour inhibitory effect (FIG. 18J) and weight change (FIG. 18K) of mice bearing subcutaneous murine Lewis lung tumour over 21 d after initiation of treatment (n=8 per group); Treatment protocol followed experiment design in FIG. 18A, 5 cycles (i.v. regimen 4-6 and i.p. anti-PD-1. Data are presented as mean±s.d. FIG. 18L shows cumulative survival of different treatment groups of mice bearing murine Lewis lung tumours. Statistical significance was calculated using a two-sided unpaired t test; *P<0.05, **P<0.01, ***P<0.001.

FIG. 19A shows structure of CPTNPs (BP-k-l-v-f-f-k-(r)₈) where Green—Bispyrene. Blue—hydrophobic bonding motif. Red—Cell-penetrating peptide. FIG. 19B shows GG-CPTNP (BP-k-l-v-g-g-k-(r)₈) with similar coloration to A where the duel phenylalanine motif is replaced with a duel glycine motif. FIG. 19C shows DLS of CPTNPs (FF) and GG-CPTNPs (GG) in various pH. FIG. 19D shows fluorescence of CPTNP nanoparticles and CPTNP monomers where the AIEE effect of BP may be observed. FIG. 19E shows Zeta potential of FF and GG CPTNPs measured at 50 μM. (a:b, p<0.0005) FIG. 19F shows TEM images of CPTNPs in various specified environments. Scale bar is 100 μm in each image.

FIG. 20 shows Chemical structure and mass spectra via MALDI-TOF of transformable peptide monomer (TPM) 1 LXY30-KLVFFK(Pa), 2 proLLP2A-KLVFFK(R848), 3 LXY30-KAAGGK(Pa), 4 proLLP2A-KAAGGK(R848). Experiments were repeated three times.

FIG. 21A shows TEM images and size distribution of NPsTPM1, NPsTPM1 and T-NPs at the H2O and DMSO ratio of 99:1. Experiments were repeated three times. FIG. 21B shows the critical aggregation concentration (CAC) of T-NPs was measured by using pyrene as a probe. Experiments were repeated three times. FIG. 21C shows nanoparticle stability of T-NPs in serum and protease (PBS solution of pH 7.4 with/without 10% FBS and protease) at 37° C. was measured by dynamic light scattering. Data are presented as the mean±s.d., n=3 independent experiments. FIG. 21D shows TEM images of freshly prepared T-NPs and T-NPs after 24 h in PBS solution. Experiments were repeated three times. FIG. 21E show Tte CAC of T-NFs was measured by using pyrene as a probe. Experiments were repeated three times. The scale bar in all TEM images is 100 nm. The concentration of T-NPs used in FIGS. 21A, 21C, and 21D was 20 μM.

FIG. 22 shows TEM images of initial UT-NPs and UT-NPs interaction with α₃β₁ integrin protein for 24 h. The molar ratio of α₃β₁ integrin protein/peptide ligand was approximately 1:1000. The scale bar is 100 nm. The concentration used in the experiment was 20 μM. Experiments were repeated three times.

FIG. 23 shows biotinylated LXY30 peptide (blue curve) and negative control (red curve) incubation with 4T1 cells were analyzed with flow cytometry. Experiments were repeated three times. 3×105 cells incubated with 1 μM biotinylated LXY30 for 30 min on ice, after washing with PBS followed by incubation with 1:500 streptavidin-PE (1 mg/mL) for 30 min, then run with flow cytometry.

FIG. 24 shows viability of 4T1 cells after incubation with T-NPs and UT-NPs at different concentrations for 48 h. Data are presented as mean±s.d., n=3 independent experiments.

FIG. 25 shows blood test parameters in terms of red blood cells (RBC), white blood cells (WBC), platelets, hemoglobin, lymphocyte and total protein of healthy Balb/c mice, after 8 q.o.d. intravenous injections of T-NPs and UT-NPs (13 mg/kg per injection). Data are presented as the mean±s.d., n=3 independent experiments.

FIG. 26 shows blood test parameters in terms of liver function creatinine, alanine transaminase, aspartate transaminase, albumin, alkaline phosphatase, total bilirubin of healthy Balb/c mice after 8 q.o.d. intravenous injection of T-NPs and UT-NPs (13 mg/kg per injection). Data are presented as the mean±s.d., n=3 independent experiments.

FIG. 27 shows in vivo blood pharmacokinetics and parameter of T-NPs and UT-NPs (Data are presented as the mean±s.d., n=3 independent experiments). The C-max, AUC and T1/2 (hours) were calculated by Kinetica 5.0.

### DETAILED DESCRIPTION OF THE INVENTION

### I. General

The present invention provides compounds comprising a hydrophobic moiety, a beta-sheet peptide, and a hydrophilic targeting ligand, which can form nanocarriers. The nanocarriers can comprise a plurality of one conjugate or two different conjugates. The nanocarriers can transform in situ to form nanofibrils for treatment of diseases and imaging.

### II. Definitions

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

“A,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

“Hydrophobic moiety” refers to the part of the compound which is substantially insoluble in water. For example, when a plurality of compounds are present which comprise a hydrophobic and hydrophilic moiety, the hydrophobic moiety will orient themselves in such a way as to avoid and minimize interaction with water molecules. Hydrophobicity of a moiety can be determined by one of ordinary skill in the art by using the octanol-water reference system to measure the logarithm of the partition coefficient (log P value). Log P values greater than 0 indicate the compound is hydrophobic, with greater values indicating greater hydrophobicity.

“Peptide” refers to a compound comprising two or more amino acids covalently linked by peptide bonds. As used herein, the term includes amino acid chains of any length, including full-length proteins.

“Beta-sheet”, also known as beta-pleated sheet, refers to the secondary structure in proteins and comprises beta strands stabilized by hydrogen bonds. Beta-strands can stack parallel or antiparallel to each other to form beta-sheets.

“Beta-sheet peptide domain” refers to a domain within a protein structure comprising beta-sheets.

“Beta-amyloid peptide” refers to peptides that form amyloid plaques in the brain. The formation of amyloid plaques in the brain is found in subjects with Alzheimer's disease.

“Hydrophilic targeting ligand” refers to a portion of the compound that can target cell surface receptors, cell surface proteins, or extracellular components and are hydrophilic. Hydrophilicity can be determined by measuring the log P value of a compound, wherein values less than 0 indicate hydrophilicity. Lower values indicate higher hydrophilicity. Targeting ligands can be used to target transmembrane receptors such as, but not limited to integrins and epidermal growth factor receptors, to delivery compounds, drugs, or components of interest to the cell or extracellular environment. Hydrophilic targeting ligands can include, but are not limited to peptides.

“Prodrug” refers to a compound that is biologically inactive, which becomes biologically active after being metabolized in situ. The prodrug can be metabolized by spontaneous reactions or enzymes within a mammal, resulting in an active compound. Functional groups useful in prodrugs include, but are not limited to esters, amides, carbamates, oximes, imines, ethers, phosphates, or beta-amino-ketones.

“LLP2A”, “LXY30”, and “LXW64” refer to compounds that can bind to an integrin protein. The structures of the three individual compounds are known by one of skill in the art.

“DUPA” refers to a glutamate urea compound and can be used to deliver cytotoxic drugs to prostate cancer cells. DUPA, 2-[3-(1,3-dicarboxypropyl)ureido]pentanedioic acid, has the following structure:

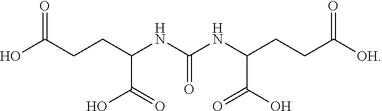 [SMILES: O=C(O)CCC(NC(=O)NC(CCC(=O)O)C(=O)O)C(=O)O]

“LHRH peptide” refers to luteinizing hormone releasing hormone peptide, and is commercially available. LHRH peptide can be used to target ovarian and prostate cancer cells.

“HER2 ligand” refers to a ligand that can bind to the HER2 protein. Examples include, but are not limited to anti-HER2 monoclonal antibodies, such as, but not limited to trastuzumab and pertuzumab and the EGFR ligands listed below.

“EGFR ligand” refers to a ligand that can bind to the EGFR protein. Examples include, but are not limited to EGF, TGF-alpha, HB-EGF, amhiregulin, betacellulin, epigen, epiregulin, neuregulin 1, neuregulin 2, neuregulin 3, and neuregulin 4.

“Toll-like receptor agonist” refers to a compound that binds to the toll-like receptor on cells, which plays a key role in the immune system. Binding to the receptor can activate the receptor to produce a biological response. An example of a toll-like receptor agonist includes, but is not limited to CpG oligonucleotides.

“CpG oligonucleotides”, also known as CpG ODN, refer to cytosine-guanosine dinucleotide motifs. The two nucleotides can be linked by a phosphodiester linker, or a modified phosphorothioate linker.

“Dye” or “fluorescent dye” refers to a chemical molecule which emits lights, commonly in the 300-700 nm range, after excitation of the chemical molecule. Upon absorption of transferred light energy (e.g., photon), a dye molecule goes into an excited state. As the molecule exits the excited state, it emits the light energy in the form of lower energy photon (e.g., emits fluorescence) and returns the dye molecule to its ground state. A dye can be a natural chemical compound or a synthetic chemical compound. Dyes include, but are not limited to cyanines, porphyrins, and bis-pyrenes.

“Porphyrin” refers to any compound, with the following porphin core:

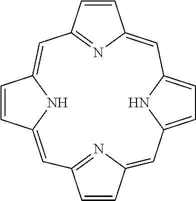 [SMILES: C1=Cc2cc3ccc(cc4nc(cc5ccc(cc1n2)[nH]5)C=C4)[nH]3]

wherein the porphin core can be substituted or unsubstituted.

“Bis-pyrene” refers to a compound which comprises two pyrene subunits covalently linked to each other. The two pyrene subunits can be linked directly or through a linker. The linker can be any linker known to one of skill in the art, such as but not limited to, alkylenes, alkenylenes, alkynylenes, aryls, heteroaryls, aryl ketones, ketones, amines, amides, and ureas, wherein the linker can be substituted.

“Radiometal chelator” refers to a polydentate ligand binding to a single central metal atom or ion. The metal atom or ion can be a radioactive isotope of the metal. Radiometal chelators include, but are not limited to Gd(III) chelators, DOTA chelator and NOTA chelator. Gd(III) chelators include, but are not limited to gadopentetic acid, gadoteric acid, gadodiamide, gadobenic acid, gadoteridol, gadoversetamide, and gadobutrol.

“Cyanine” or “cyanine dye” refers to a synthetic dye family belonging to a polymethine group. Cyanines can be used as fluorescent dyes for biomedical imaging. Cyanines can be streptocyanines (also known as open chain cyanines), hemicyanines, and closed chain cyanines. Closed chain cyanines have nitrogens which are each independently part of a heteroaromatic moiety.

“Drug” refers to an agent capable of treating and/or ameliorating a condition or disease. A drug may be a hydrophobic drug, which is any drug that repels water. Hydrophobic drugs useful in the present invention include, but are not limited to, deoxycholic acid, taxanes, doxorubicin, etoposide, irinotecan, SN-38, cyclosporin A, podophyllotoxin, Carmustine, Amphotericin, Ixabepilone, Patupilone (epothelone class), rapamycin and platinum drugs. Other drugs includes non-steroidal anti-inflammatory drugs, and vinca alkaloids such as vinblastine and vincristine. The drugs of the present invention also include prodrug forms. One of skill in the art will appreciate that other drugs are useful in the present invention.

“Chemotherapeutic agent” refers to chemical drugs that can be used in the treatment of diseases such as, but not limited to, cancers, tumors and neoplasms. In some embodiments, a chemotherapeutic agent can be in the form of a prodrug which can be activated to a cytotoxic form. Chemotherapeutic agents commonly known by one of ordinary skill in the art can be used in the present invention. Chemotherapeutic agents include, but are not limited to resiquimod, gardiquimod, and imiquimod.

“Immunomodulatory agent” refers to a type of drug which can modify immune responses by stimulating or suppressing the immune system. Immunomodulatory agents include, but are not limited to resiquimod, gardiquimod, and imiquimod.

“Anti-HER2 rhumAb 4D5” refers to a type of HER2 antibody, and is also known as trastuzumab. Trastuzumab is commonly used to treat breast and stomach cancer and is commercially available. Trastuzumab comprises at least 50% peptide sequence identity of SEQ ID NO: 4. The peptide sequence of trastuzumab is described in “Rationally designed anti-HER2/neu peptide mimetic disables P185HER2/neu tyrosine kinases in vitro and in vivo” (Park et al. Nat Biotechnol. 2000 February; 18(2):194-8.)

“CDR-H3 loop” refers to a region inside a HER2 antibody involved with antigen binding.

“Nanocarrier” or “nanoparticle” refers to a micelle resulting from aggregation of the compounds of the invention. The nanocarrier of the present invention can have a hydrophobic core and a hydrophilic exterior.

“Nanofibrils” refer to tubular, rod-like fibrils which have a diameter ranging from tens to hundreds of nanometers. Nanofibrils can have high length-to-diameter ratios. Nanofibrils of the present invention can be formed by an in situ transformation of the nanoparticles after binding at the targeted site.

“Fibrillary structures” refer to linear, rod-like fibrils with diameters on the order of nanometers to micrometers and have a high length-to-diameter ratio. Fibrillary structures may include biopolymers. Fibrillary structures include, but are not limited to, nanofibrils and microfibrils.

“Cell surface” refers to the plasma membrane, which separates the extracellular space from the interior of the cell. The cell surface comprises the lipid bilayer, proteins, and carbohydrates.

“Acellular component” refers to the extracellular environment of a cell and includes, but is not limited to the extracellular matrix, extracellular vesicles, and cytokines surround a cell. The extracellular matrix comprises collagens, fibronectin, and other matrix proteins. Ligands and compounds can interact with an acellular component of cancerous cells to affect the growth of cancer cells.

“Tumor microenvironment” refers to tumor cells and the acellular environment surrounding it, including, but not limited to the extracellular matrix, signaling molecules, immune cells, stromal cells, vasculature, blood vessels, cytokines, chemokines, growth factors, and fibroblasts. Tumors can interact with the surround cells in the microenvironment through the lymphatic and circulatory systems to affect the growth and evolution of cancer cells.

“Treat”, “treating” and “treatment” refers to any indicia of success in the treatment or amelioration of an injury, pathology, condition, or symptom (e.g., pain), including any objective or subjective parameter such as abatement; remission; diminishing of symptoms or making the symptom, injury, pathology or condition more tolerable to the patient; decreasing the frequency or duration of the symptom or condition; or, in some situations, preventing the onset of the symptom. The treatment or amelioration of symptoms can be based on any objective or subjective parameter; including, e.g., the result of a physical examination.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

“Subject” refers to animals such as mammals, including, but not limited to, primates (e.g., humans), cows, sheep, goats, horses, dogs, cats, rabbits, rats, mice and the like. In certain embodiments, the subject is a human.

“Therapeutically effective amount” or “therapeutically sufficient amount” or “effective or sufficient amount” refers to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can often be lower than the conventional therapeutically effective dose for non-sensitized cells.

“Cancer” refers to diseases with abnormal cell growth and divides without control. Cancer cells can spread locally or through the bloodstream and lymphatic system to other parts of the body. The term is also intended to include any disease of an organ or tissue characterized by poorly controlled or uncontrolled multiplication of normal or abnormal cells in that tissue and its effect on the body as a whole.

“Imaging” refers to using a device outside of the subject to determine the location of an imaging agent, such as a compound of the present invention. Examples of imaging tools include, but are not limited to, fluorescence microscopy, positron emission tomography (PET), magnetic resonance imaging (MRI), ultrasound, single photon emission computed tomography (SPECT) and x-ray computed tomography (CT). The positron emission tomography detects radiation from the emission of positrons by an imaging agent.

### III. Compounds

In some embodiments, the present invention provides a compound of formula (I): A-B-C (I), wherein A is a hydrophobic moiety; B is a peptide, wherein the peptide forms a beta-sheet; and C is a hydrophilic targeting ligand. The hydrophilic targeting ligand can include a HER2 ligand, and any other suitable target ligand.

In some embodiments, the present invention provides a compound of formula (I): A-B-C (I), wherein A is a hydrophobic moiety; B is a peptide, wherein the peptide forms a beta-sheet; and C is a hydrophilic targeting ligand, wherein the hydrophilic targeting ligand is a LLP2A prodrug, LLP2A, LXY30, LXW64, DUPA, folate, a LHRH peptide, a HER2 ligand, an EGFR ligand, or a toll-like receptor agonist CpG oligonucleotides.

In some embodiments, the present invention provides a compound of formula (I) wherein A is bis-pyrene; B is a peptide, wherein the peptide forms a beta-sheet; and C is a HER2 ligand.

In some embodiments, the present invention provides a compound of formula (I): A-B-C (I), wherein A is a hydrophobic moiety; B is a peptide, wherein the peptide forms a beta-sheet; and C is a hydrophilic targeting ligand, wherein when the hydrophobic moiety is bis-pyrene, then C is other than a HER2 ligand.

In some embodiments, the present invention provides a compound of formula (I): A-B-C (I), wherein A is a hydrophobic moiety; B is a peptide, wherein the peptide forms a beta-sheet; and C is a hydrophilic targeting ligand, wherein the hydrophilic targeting ligand is a LLP2A prodrug, LLP2A, LXY30, LXW64, DUPA, folate, a LHRH peptide, a HER2 ligand, an EGFR ligand, or a toll-like receptor agonist CpG oligonucleotides and wherein when the hydrophobic moiety is bis-pyrene, then C is a LLP2A prodrug, LLP2A, LXY30, LXW64, DUPA, folate, a LHRH peptide, an EGFR ligand, or a toll-like receptor agonist CpG oligonucleotides.

In some embodiments, the present invention provides a compound of formula (I): A-B-C (I), wherein A is a hydrophobic moiety; B is a peptide, wherein the peptide forms a beta-sheet; and C is a hydrophilic targeting ligand, wherein the hydrophilic targeting ligand is a LLP2A prodrug, LLP2A, LXY30, LXW64, DUPA, a LHRH peptide, a HER2 ligand, an EGFR ligand, or a toll-like receptor agonist CpG oligonucleotides and wherein when the hydrophobic moiety is bis-pyrene, then C is a LLP2A prodrug, LLP2A, LXY30, LXW64, DUPA, a LHRH peptide, an EGFR ligand, or a toll-like receptor agonist CpG oligonucleotides.

In some embodiments, the present invention provides a compound of formula (I): A-B-C (I), wherein A is a hydrophobic moiety; B is a peptide, wherein the peptide forms a beta-sheet; and C is a hydrophilic targeting ligand, wherein the hydrophilic targeting ligand is a LLP2A prodrug, LLP2A, LXY30, LXW64, DUPA, folate, a LHRH peptide, a HER2 ligand, an EGFR ligand, or a toll-like receptor agonist CpG oligonucleotides and wherein when the hydrophobic moiety is bis-pyrene, then C is a LLP2A prodrug, LLP2A, LXY30, LXW64, DUPA, folate, a LHRH peptide, an EGFR ligand, or a toll-like receptor agonist CpG oligonucleotides.

Hydrophobic moieties useful in the present invention includes any suitable hydrophobic moiety known by one of skill in the art. Hydrophobicity and hydrophilicity are commonly measured by the log P values of the compounds using the octane-water reference system. Values lower than 0 indicate hydrophilicity whereas values higher than 0 indicate hydrophobicity. Hydrophobic moieties useful in the present invention includes moieties with log P values of at least 1. In some embodiments, hydrophobic moieties useful in the present invention have a log P value of at least 1.5. In some embodiments, hydrophobic moieties useful in the present invention have a log P value of 1.5-15. Hydrophobic moieties include, but are not limited to cholesterol, vitamin D, vitamin D derivatives, vitamin E, vitamin E derivatives, dyes, drugs, and radiometal chelators. In some embodiments, the hydrophobic moiety is cholesterol, vitamin D, vitamin D derivatives, vitamin E, vitamin E derivatives, a dye, or a drug. In some embodiments, the hydrophobic moiety is cholesterol, vitamin D, vitamin E, a dye, or a drug. In some embodiments, the hydrophobic moiety is cholesterol, vitamin D, or vitamin E. In some embodiments, the hydrophobic moiety is a dye or drug.

Dyes useful in the present invention include any dye described in, but not limited to, Johnson, I., Histochemical Journal, 20:123-140 (1998), and The Molecular Probes® Handbook, 11^{th} Edition, ed. Johnson and Spence, Life Technologies, Carlsbad, Calif., 2010. The dyes can be fluorescent dyes, triarylmethane dyes, cyanine dyes, benzylidene imidazolinone dyes, indigo dyes, bis-pyrenes and porphyrins. In some embodiments, the hydrophobic moiety is a dye. In some embodiments, the hydrophobic moiety is a fluorescent dye, porphyrin, or bis-pyrene. In some embodiments, the hydrophobic moiety is a cyanine dye, porphyrin, or bis-pyrene.

Drugs useful in the present invention include chemotherapeutic agents and immunomodulatory agents. For example, the drugs can be, but are not limited to, deoxycholic acid, or the salt form deoxycholate, pembrolizumab, nivolumab, cemiplimab, a taxane (e.g., paclitaxel, docetaxel, cabazitaxel, Baccatin III, 10-deacetylbaccatin, Hongdoushan A, Hongdoushan B, or Hongdoushan C), doxorubicin, etoposide, irinotecan, SN-38, cyclosporin A, podophyllotoxin, Carmustine, Amphotericin, Ixabepilone, Patupilone (epothelone class), rapamycin and platinum drugs. Other drugs include non-steroidal anti-inflammatory drugs, and vinca alkaloids such as vinblastine and vincristine. In some embodiments, the drug is paclitaxel, resiquimod, gardiquimod, or deoxycholate.

In some embodiments, the hydrophobic moiety is a chemotherapeutic agent, a fluorescent dye, an immunomodulatory agent, a toll-like receptor agonist, a small molecule agonist of stimulator of interferon gene (STING), porphyrin, deoxycholate, cholesterol, vitamin D, or vitamin E. In some embodiments, the hydrophobic moiety is a chemotherapeutic agent, a fluorescent dye, an immunomodulatory agent, a small molecule agonist of stimulator of interferon gene (STING), porphyrin, cholesterol, vitamin D, or vitamin E. In some embodiments, the hydrophobic moiety is a chemotherapeutic agent, a fluorescent dye, an immunomodulatory agent, a small molecule agonist of stimulator of interferon gene (STING), porphyrin or deoxycholate. In some embodiments, the hydrophobic moiety is a chemotherapeutic agent, a fluorescent dye, an immunomodulatory agent, porphyrin or deoxycholate. In some embodiments, the hydrophobic moiety is paclitaxel, bis-pyrene, cyanine dye, resiquimod, gardiquimod, amidobenzimidazole, porphyrin, or deoxycholate. In some embodiments, the hydrophobic moiety is paclitaxel, bis-pyrene, cyanine dye, resiquimod, gardiquimod, porphyrin, or deoxycholate. In some embodiments, the hydrophobic moiety is resiquimod or porphyrin.

Porphyrins useful in the present invention include any porphyrin known by one of skill in the art. In some embodiments, the porphyrin is a substituted or unsubstituted porphin, protoporphyrin IX, octaethylporphyrin, tetraphenyl porphyrin, pyropheophorbide-a, pheophorbide, chlorin e6, purpurin or purpurinimide. In some embodiments, the porphyrin is pyropheophorbide-a, pheophorbide, chlorin e6, purpurin or purpurinimide. In some embodiments, the porphyrin is pheophorbide-a. In some embodiments, the porphyrin has the following structure:

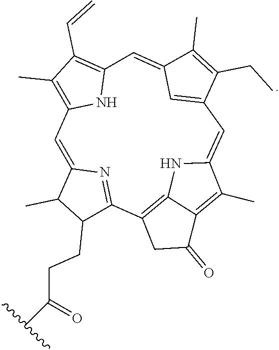 [SMILES: *C(=O)CCC1c2nc(cc3[nH]c(cc4cc(cc5[nH]c6c2CC(=O)c6c5C)C(CC)=C4C)c(C=C)c3C)C1C]

In some embodiments, the hydrophobic moiety is bis-pyrene. Bis-pyrenes useful in the present invention include any bis-pyrene known by one of skill in the art. In some embodiments, the bis-pyrene comprises the following moieties:

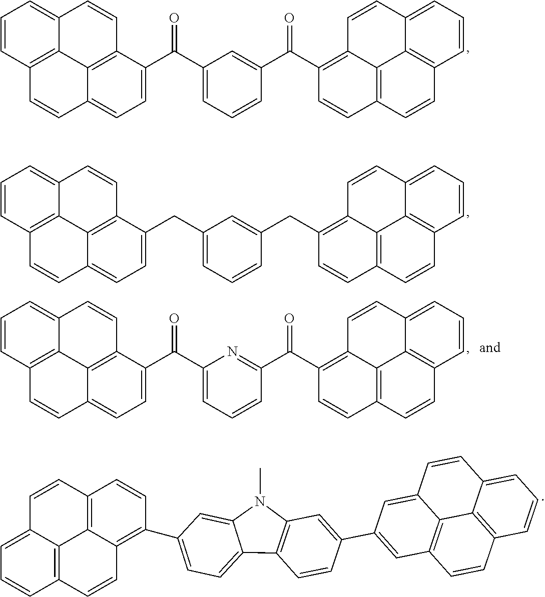

In some embodiments, the bis-pyrene comprises the following:

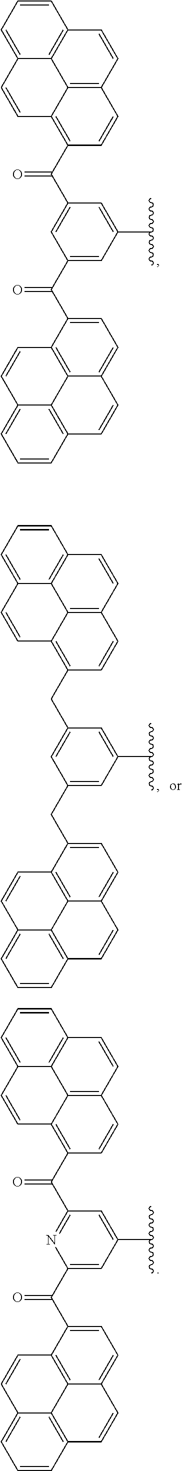

In some embodiments, the bis-pyrene has the following structure:

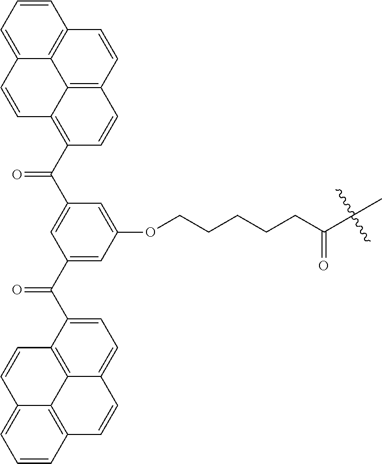 [SMILES: *C(=O)CCCCCOc1cc(C(=O)c2ccc3ccc4cccc5ccc2c3c45)cc(C(=O)c2ccc3ccc4cccc5ccc2c3c45)c1]

The peptides useful in the present invention can be any suitable peptide, and have any suitable peptide sequence length known by one of skill in the art. In some embodiments, the peptide is a peptide sequence 5-50 amino acids in length. In some embodiments, the peptide is a peptide sequence 5-40 amino acids in length. In some embodiments, the peptide is a peptide sequence 5-30 amino acids in length. In some embodiments, the peptide is a peptide sequence 5-25 amino acids in length. In some embodiments, the peptide is a peptide sequence 5-20 amino acids in length. In some embodiments, the peptide is a peptide sequence 5-15 amino acids in length. In some embodiments, the peptide is a peptide sequence about 5-10 amino acids in length.

Adjacent beta-strand peptides form hydrogen bonds between each strand resulting in beta sheet peptides. The beta-sheet peptide sequences useful in the present invention can be any suitable peptide sequence known by one of skill in the art. For example, commonly known beta-sheet peptides are described in “Branched KLVFF tetramers strongly potentiate inhibition of beta-amyloid aggregation” (Chafekar et al., Chembiochem. 2007 Oct. 15; 8(15):1857-64). In some embodiments, the peptide comprises a peptide sequence from a beta-sheet peptide domain of green fluorescent protein, interleukins, immunoglobulins, or beta-amyloid peptide. In some embodiments, the peptide comprises a peptide sequence from a beta-sheet peptide domain of a beta-amyloid peptide. In some embodiments, the beta-amyloid peptide is beta-amyloid 40 or beta-amyloid 42. In some embodiments, the beta-amyloid peptide is beta-amyloid 40.

In some embodiments, the peptide comprises at least 40% sequence identity to SEQ ID NO:1. In some embodiments, the peptide comprises at least 50% sequence identity to SEQ ID NO:1. In some embodiments, the peptide comprises at least 60% sequence identity to SEQ ID NO:1. In some embodiments, the peptide comprises at least 80% sequence identity to SEQ ID NO:1. In some embodiments, the peptide comprises SEQ ID NO:1.

In some embodiments, the peptide comprises at least 40% sequence identity to SEQ ID NO:2. In some embodiments, the peptide comprises at least 50% sequence identity to SEQ ID NO:2. In some embodiments, the peptide comprises at least 60% sequence identity to SEQ ID NO:2. In some embodiments, the peptide comprises at least 80% sequence identity to SEQ ID NO:2. In some embodiments, the peptide comprises SEQ ID NO:2.

In some embodiments, the peptide comprises at least 40% sequence identity to SEQ ID NO:3. In some embodiments, the peptide comprises at least 50% sequence identity to SEQ ID NO:3. In some embodiments, the peptide comprises at least 60% sequence identity to SEQ ID NO:3. In some embodiments, the peptide comprises at least 80% sequence identity to SEQ ID NO:3. In some embodiments, the peptide comprises SEQ ID NO:3.

Hydrophilic targeting ligands useful in the present invention can target receptors on the cell surface, or the acellular component of the tumor microenvironment. Hydrophilicity and hydrophobicity are commonly measured by the log P values of the compounds using the octane-water reference system. Values lower than 0 indicate hydrophilicity whereas values higher than 0 indicate hydrophobicity. In some embodiments, the hydrophilic targeting ligand includes peptides which target cell surface receptors or acellular components in the tumor microenvironment, which includes, but is not limited to immune cells such as macrophages, T cells, and B cells. In some embodiments, the hydrophilic targeting ligand targets cell surface receptors such as, but not limited to, integrins and epidermal growth factor receptors. In some embodiments, the hydrophilic targeting ligand targets integrins, epidermal growth factors, and toll-like receptors.

In some embodiments, the hydrophilic targeting ligand is a HER2 ligand, a prodrug for a HER2 ligand, a receptor tyrosine-protein kinase-targeting ligand, an integrin-targeting ligand, epidermal growth factor receptor-targeting ligand, ovarian cancer cell-targeting ligand, or prostate cancer cell-targeting ligand. In some embodiments, the hydrophilic targeting ligand is a HER2 ligand, a prodrug for a HER2 ligand, an integrin-targeting ligand, epidermal growth factor receptor-targeting ligand, ovarian cancer cell targeting ligand, or prostate cancer cell targeting ligand.

In some embodiments, the hydrophilic targeting ligand is a HER2 ligand. In some embodiments, the HER2 ligand is an anti-HER2 antibody peptide. In some embodiments, the hydrophilic targeting ligand is the HER2 ligand, wherein the HER2 ligand is an anti-HER2 antibody peptide mimic derived from the primary sequence of the CDR-H3 loop of the anti-HER2 rhumAb 4D5. In some embodiments, the HER2 ligand is as described in “Rationally designed anti-HER2/neu peptide mimetic disables P185HER2/neu tyrosine kinases in vitro and in vivo” (Park et al. Nat Biotechnol. 2000 February; 18(2):194-8.)

In some embodiments, the HER2 ligand has at least 40% sequence identity to SEQ ID NO:4. In some embodiments, the HER2 ligand has at least 50% sequence identity to SEQ ID NO:4. In some embodiments, the HER2 ligand has at least 60% sequence identity to SEQ ID NO:4. In some embodiments, the HER2 ligand has at least 80% sequence identity to SEQ ID NO:4. In some embodiments, the HER2 ligand is SEQ ID NO:4.

In some embodiments, the hydrophilic targeting ligand is an integrin-targeting ligand, epidermal growth factor receptor-targeting ligand, ovarian cancer cell targeting ligand, or prostate cancer cell targeting ligand. In some embodiments, the hydrophilic targeting ligand is a prodrug for an integrin-targeting ligand, epidermal growth factor receptor-targeting ligand, ovarian cancer cell targeting ligand, or prostate cancer cell targeting ligand.

In some embodiments, the hydrophilic targeting ligand is a LLP2A prodrug, LLP2A, LXY30, DUPA, folate, a LHRH peptide, or an EGFR ligand. Any one of the carboxylic acid groups in the DUPA structure can be used to link to the beta-sheet peptide. LHRH analog peptide comprises the following peptide sequence: H-Glp-His-Trp-Ser-Thr-Lys-Leu-Arg-Pro-Gly-NH₂ or H-Glp-His-Trp-Ser-His-Asp-Trp-Lys-Pro-Gly-NH₂. The Lys side chain NH2 group of the LHRH peptides can be used to link to the beta-peptide sheet. In some embodiments, the NH₂ group is used to covalently link to the beta-peptide sheet.

EGFR ligands useful in the present invention includes any EGFR ligand known by one of skill in the art. In some embodiments, the EGFR ligand can be EGF, TGF-alpha, HB-EGF, amphiregulin, betacellulin, epigen, epiregulin, neuregulin 1, neuregulin 2, neuregulin 3, and neuregulin 4.

In some embodiments, the hydrophilic targeting ligand is a LLP2A prodrug, LLP2A, or LXY30. The LLP2A prodrug can include any cleavable functional group to be metabolized in situ known by one of skill in the art. In some embodiments, the LLP2A prodrug comprises an ester, amide, carbamate, oxime, imine, ether, phosphate, or beta-amino-ketone functional group. In some embodiments, the LLP2A prodrug comprises an ester, amide, carbamate, ether, or phosphate functional group. In some embodiments, the LLP2A prodrug comprises an ester, amide, carbamate or phosphate functional group. In some embodiments, the LLP2A prodrug comprises an ester group.

In some embodiments, the hydrophilic targeting ligand is a LLP2A prodrug, with the following structure:

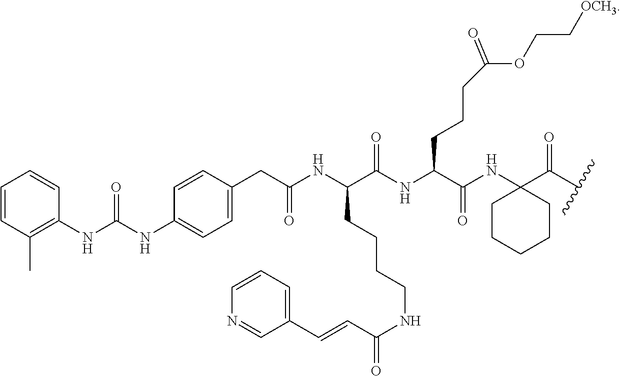 [SMILES: *C(=O)C1(NC(=O)[C@H](CCCC(=O)OCCOC)NC(=O)[C@@H](CCCCNC(=O)/C=C/c2cccnc2)NC(=O)Cc2ccc(NC(=O)Nc3ccccc3C)cc2)CCCCC1]

In some embodiments, the hydrophilic targeting ligand is LLP2A, with the following structure:

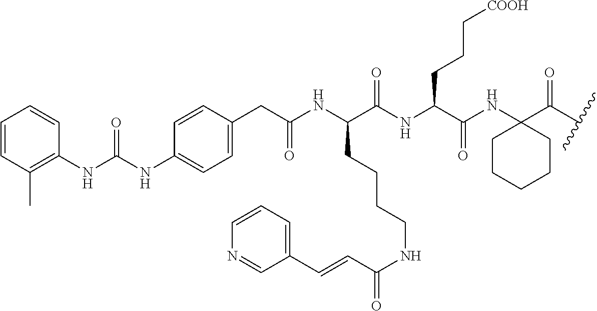 [SMILES: *C(=O)C1(NC(=O)[C@H](CCCC(=O)O)NC(=O)[C@@H](CCCCNC(=O)/C=C/c2cccnc2)NC(=O)Cc2ccc(NC(=O)Nc3ccccc3C)cc2)CCCCC1]

In some embodiments, the hydrophilic targeting ligand is LXY30, with the following structure:

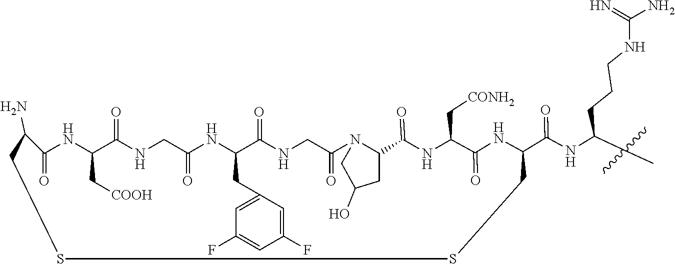

In some embodiments, the compound of the present invention has the following structure:

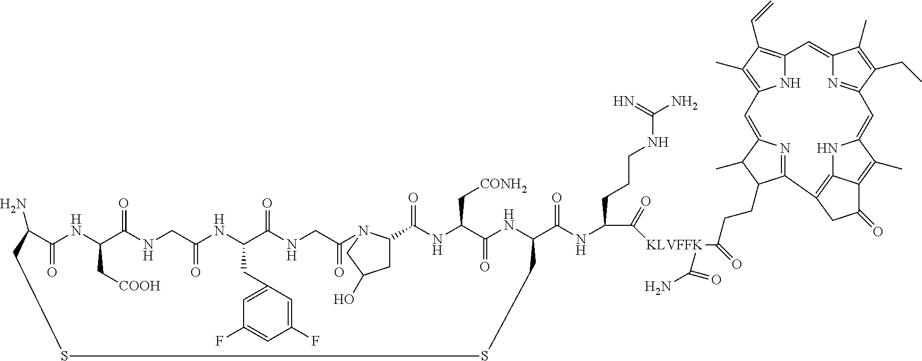

In some embodiments, the compound of the present invention has the following structure:

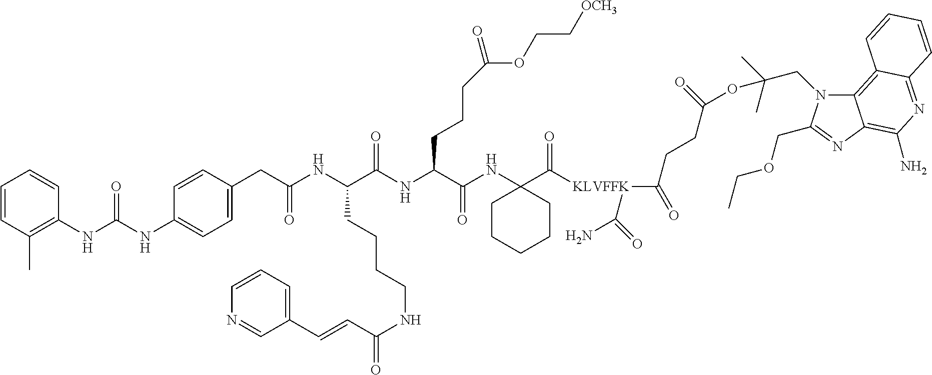

In some embodiments, the compound of the present invention has the following structure:

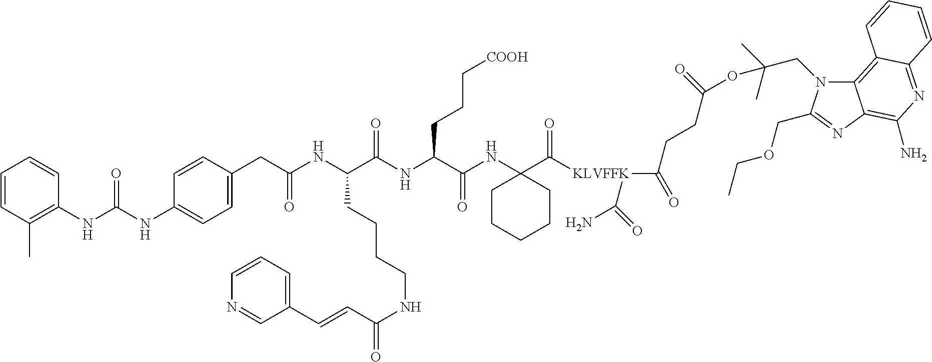

In some embodiments, the compound of the present invention has the following structure:

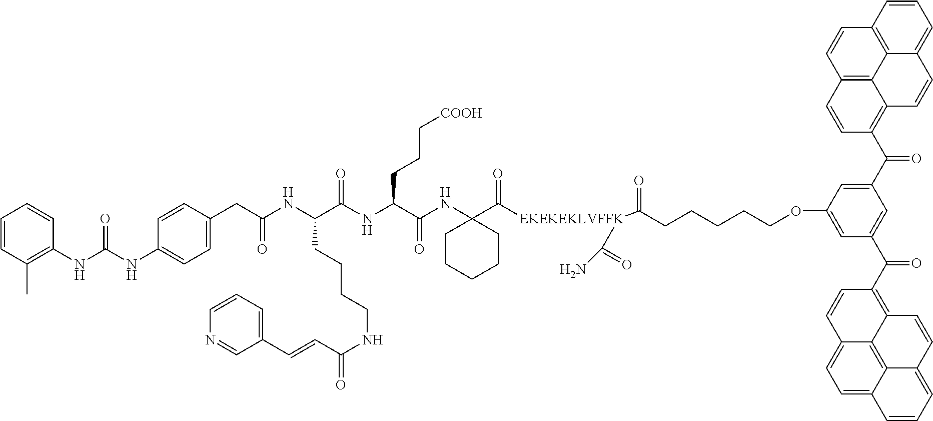

In some embodiments, the compound of the present invention has the following structure:

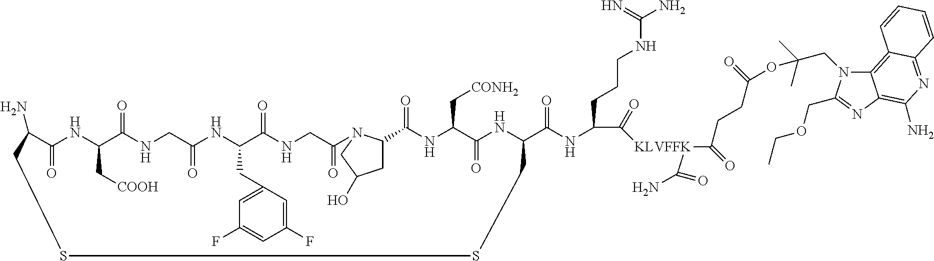

In some embodiments, the compound of the present invention has the following structure:

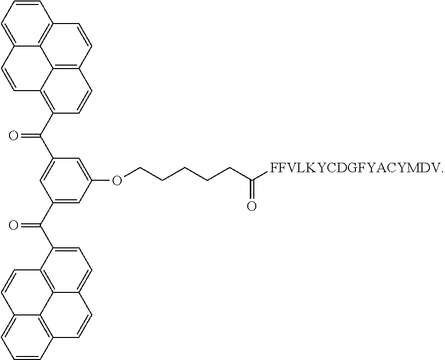

### IV. Nanocarriers

In some embodiments, the present invention provides a nanocarrier having an interior and an exterior, the nanocarrier comprising a plurality of compounds of the present invention, wherein each compound self-assembles in an aqueous solvent to form the nanocarrier such that a hydrophobic pocket is formed in the interior of the nanocarrier, and a hydrophilic group self-assembles on the exterior of the nanocarrier.

The diameter of the nanocarrier of the present invention can be any suitable size known by one of skill in the art. In some embodiments, the nanocarrier can have a diameter of 5 to 100 nm. In some embodiments, the nanocarrier can have a diameter of 10 to 100 nm. In some embodiments, the nanocarrier can have a diameter of 15 to 80 nm. In some embodiments, the nanocarrier can have a diameter of 25 to 60 nm. In some embodiments, the nanocarrier can have a diameter of about 20 nm, 30 nm, 40 nm, 50 nm, 60 nm, or about 70 nm. In some embodiments, the nanocarrier can have a diameter of about 20 nm or about 30 nm. In some embodiments, the nanocarrier can have a diameter of about 20 nm. In some embodiments, the nanocarrier can have a diameter of about 30 nm.

The exterior of the nanocarrier can be used for cell targeting. The nanocarrier of the present invention can target cell surface receptors and proteins such as, but not limited to integrins, human epidermal growth factor receptor 2 (HER2), epidermal growth factor receptors, and G protein-coupled receptors. In some embodiments, the nanocarrier can target integrins and HER2.

The nanocarrier can transform in situ after binding to the receptors or proteins on the cell surface to form a nanofibrillar structure. In some embodiments, the nanocarrier can transform in situ after binding to HER2 on the cell surface.

In some embodiments, the nanocarrier further comprises a hydrophobic drug or an imaging agent sequestered in the hydrophobic pocket of the nanocarrier.

The hydrophobic drugs useful in the present invention can be any hydrophobic drug known by one of skill in the art. Hydrophobic drugs useful in the present invention include, but are not limited to, deoxycholic acid, deoxycholate, resiquimod, gardiquimod, imiquimod, a taxane (e.g., paclitaxel, docetaxel, cabazitaxel, Baccatin III, 10-deacetylbaccatin, Hongdoushan A, Hongdoushan B, or Hongdoushan C), doxorubicin, etoposide, irinotecan, SN-38, cyclosporin A, podophyllotoxin, Carmustine, Amphotericin, Ixabepilone, Patupilone (epothelone class), rapamycin and platinum drugs. Other drugs includes non-steroidal anti-inflammatory drugs, and vinca alkaloids such as vinblastine and vincristine.

The imaging agents useful in the present invention can be any imaging agent known by one of skill in the art. Imaging agents include, but are not limited to, paramagnetic agents, optical probes, and radionuclides. Paramagnetic agents are imaging agents that are magnetic under an externally applied field. Examples of paramagnetic agents include, but are not limited to, iron particles including nanoparticles. Optical probes are fluorescent compounds that can be detected by excitation at one wavelength of radiation and detection at a second, different, wavelength of radiation. Optical probes useful in the present invention include, but are not limited to, Cy5.5, Alexa 680, Cy5, DiD (1,1′-dioctadecyl-3,3,3′,3′-tetramethylindodicarbocyanine perchlorate) and DiR (1,1′-dioctadecyl-3,3,3′,3′-tetramethylindotricarbocyanine iodide). Other optical probes include quantum dots. Radionuclides are elements that undergo radioactive decay. Radionuclides useful in the present invention include, but are not limited to, ³H, ¹¹C, ¹³N, ¹⁸F, ¹⁹F, ⁶⁰Co, ⁶⁴Cu, ⁶⁷Cu, ⁶⁸Ga, ⁸²Rb, ⁹⁰Sr, ⁹⁰Y, ⁹⁹Tc, ^{99m}Tc, ¹¹¹In, ¹²³I, ¹²⁴I, ¹²⁵I, ¹²⁹I, ¹³¹I, ¹³⁷Cs, ¹⁷⁷Lu, ¹⁸⁶Re, ¹⁸⁸Re, ²¹¹At, Rn, Ra, Th, U, Pu and ²⁴¹Am.

The nanocarrier can include a plurality of conjugates. For example, the nanocarrier can include a plurality of two, three, four, five, six, or more, different conjugates. In some embodiments, the nanocarrier comprises a plurality of two different conjugates. In some embodiments, the nanocarrier comprises a plurality of three different conjugates. In some embodiments, the nanocarrier comprises a plurality of four different conjugates.

In some embodiments, the present invention provides a nanocarrier having an interior and an exterior, the nanocarrier comprising a plurality of a first conjugate and a second conjugate wherein the first conjugate comprises formula (I): A-B-C (I); and the second conjugate comprises formula (II): A′-B′-C′ (II) wherein: A and A′ are each independently a hydrophobic moiety; B and B′ are each independently a peptide, wherein each peptide independently forms a beta-sheet; and C and C′ are each independently a hydrophilic targeting ligands, wherein each hydrophilic targeting ligand is independently a LLP2A prodrug, LLP2A, LXY30, LXW64, DUPA, folate, a LHRH peptide, a HER2 ligand, an EGFR ligand, or a radiometal chelator; and wherein A and A′ are different hydrophobic moieties and/or C and C′ are different hydrophilic targeting ligands.

In some embodiments, the nanocarrier comprises a plurality of a first conjugate and a second conjugate as described above, and further comprises a third conjugate comprising formula (III): A″-B″-C″ (III) wherein A″ is a hydrophobic moiety, B″ is a peptide, wherein the peptide forms a beta sheet, and C″ is a hydrophilic targeting ligand, and wherein A, A′, and A″ are different hydrophobic moieties and/or C, C′, and C″ are different hydrophilic targeting ligands. In some embodiments, the nanocarrier further comprises a fourth, a fifth, or a sixth conjugate where each additional conjugate is independently of formula III.

The nanocarrier of the present invention can comprise a plurality of two different conjugates. The nanocarriers comprising a plurality of two different conjugates can have diameters as described above. The nanocarriers comprising a plurality of two different conjugates can have similar targeting and transformative properties as described above.

Suitable hydrophobic moieties for the nanocarriers of the present invention are described above. In some embodiments, each hydrophobic moiety is independently a dye, a drug, or a radiometal chelator. In some embodiments, each hydrophobic moiety is independently a bis-pyrene, porphyrin, resiquimod, or gardiquimod.

In some embodiments, each hydrophobic moiety is independently a porphyrin or resiquimod. In some embodiments, the porphyrin is pyropheophorbide-a, pheophorbide, chlorin e6, purpurin or purpurinimide. In some embodiments, the porphyrin is pheophorbide-a. In some embodiments, the porphyrin has the following structure:

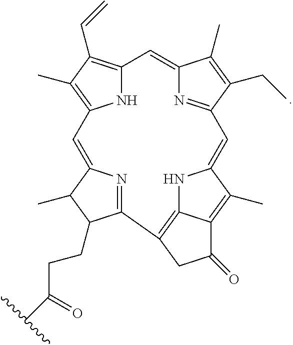 [SMILES: *C(=O)CCC1c2nc(cc3[nH]c(cc4nc(cc5[nH]c6c2CC(=O)c6c5C)C(CC)=C4C)c(C=C)c3C)C1C]

In some embodiments, the resiquimod has the following structure:

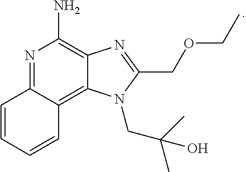 [SMILES: CCOCc1nc2c(N)nc3ccccc3c2n1CC(C)(C)O]

Radiometal chelators useful in the present invention include any radiometal chelator known by one of skill in the art. In some embodiments, the radiometal chelator is a Gd(III) chelator, diethylenetriaminepentaacetic anhydride (DTPA), 1,4,8,11-tetraazacyclotetradecane-1,4,8,11-tetraacetic acid (TETA), 1,4,7,10-tetraazacyclododecane-1,4,7,10-tetracetic acid (DOTA), or 1,4,7-triazacyclononane-1,4,7-triacetic acid (NOTA). In some embodiments, the radiometal chelator is a Gd(III) chelator, DOTA chelator, or a NOTA chelator.

Suitable peptide sequence lengths for the nanocarriers of the present invention are described above. In some embodiments, each peptide is independently a peptide sequence 5-30 amino acids in length. In some embodiments, each peptide is independently a peptide sequence 5-25 amino acids in length. In some embodiments, each peptide is independently a peptide sequence 5-20 amino acids in length.

Suitable peptide sequence for the nanocarriers of the present invention are described above. In some embodiments, each peptide independently comprises a peptide sequence from a beta-sheet peptide domain of a beta-amyloid peptide. In some embodiments, the beta-amyloid peptide is beta-amyloid 40 or beta-amyloid 42. In some embodiments, the beta-amyloid peptide is beta-amyloid 40.

In some embodiments, each peptide independently comprises at least 40% sequence identity to SEQ ID NO:1. In some embodiments, each peptide independently comprises at least 50% sequence identity to SEQ ID NO:1. In some embodiments, each peptide independently comprises at least 60% sequence identity to SEQ ID NO:1. In some embodiments, each peptide independently comprises at least 80% sequence identity to SEQ ID NO:1. In some embodiments, each peptide independently comprises SEQ ID NO:1.

In some embodiments, each peptide independently comprises at least 40% sequence identity to SEQ ID NO:2. In some embodiments, each peptide independently comprises at least 50% sequence identity to SEQ ID NO:2. In some embodiments, each peptide independently comprises at least 60% sequence identity to SEQ ID NO:2. In some embodiments, each peptide independently comprises at least 80% sequence identity to SEQ ID NO:2. In some embodiments,

Suitable hydrophilic targeting ligands for the nanocarriers of the present invention are described above. In some embodiments, each hydrophilic targeting ligand is independently a LLP2A prodrug, LLP2A, LXY30, folate, a LHRH peptide, a HER2 ligand, an EGFR ligand, a Gd(III) chelator, a DOTA chelator, or a NOTA chelator. In some embodiments, each hydrophilic targeting ligand is independently a LLP2A prodrug, LLP2A, LXY30, a LHRH peptide, a HER2 ligand, an EGFR ligand, a DOTA chelator, or a NOTA chelator. In some embodiments, each hydrophilic targeting ligand is independently a LLP2A prodrug, LLP2A or LXY30.

In some embodiments, each hydrophilic targeting ligand is independently a LLP2A prodrug, with the following structure:

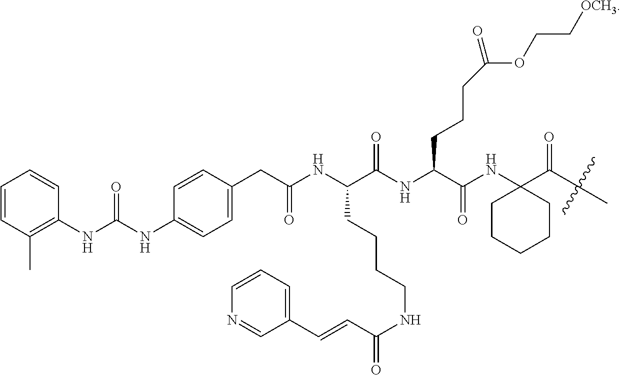 [SMILES: *C(=O)C1(NC(=O)[C@H](CCCC(=O)OCCOC)NC(=O)[C@H](CCCCNC(=O)/C=C/c2cccnc2)NC(=O)Cc2ccc(NC(=O)Nc3ccccc3C)cc2)CCCCC1]

In some embodiments, each hydrophilic targeting ligand is independently LLP2A, with the following structure:

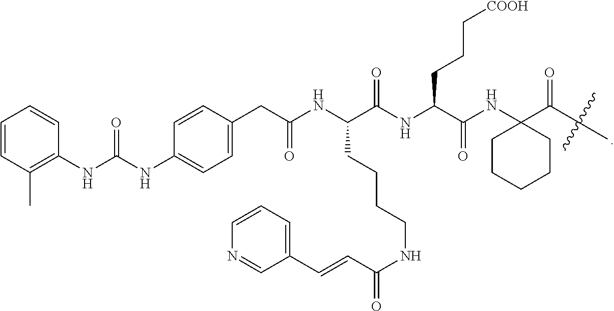 [SMILES: *C(=O)C1(NC(=O)[C@H](CCCC(=O)O)NC(=O)[C@H](CCCCNC(=O)/C=C/c2cccnc2)NC(=O)Cc2ccc(NC(=O)Nc3ccccc3C)cc2)CCCCC1]

In some embodiments, each hydrophilic targeting ligand is independently LXY30, with the following structure:

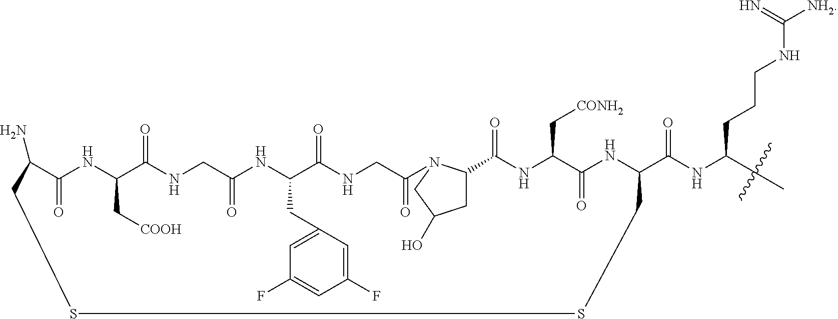

In some embodiments, the first conjugate has the structure:

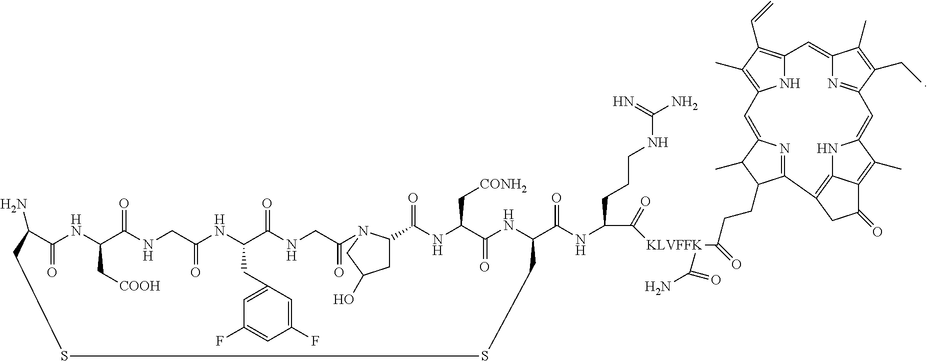

In some embodiments, the second conjugate has the structure:

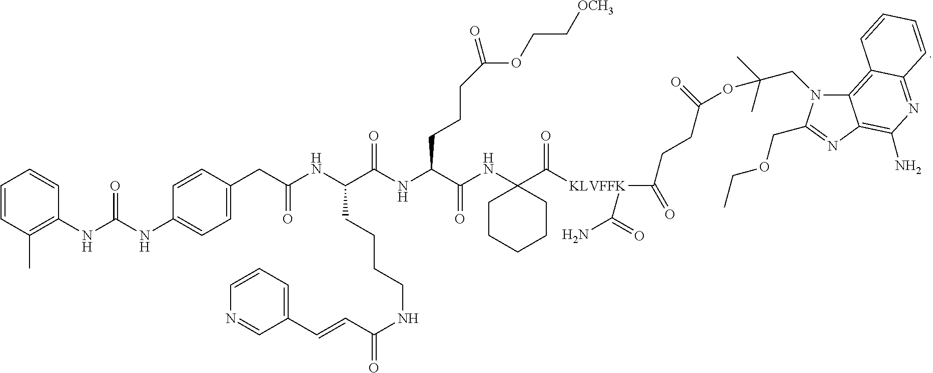

In some embodiments, the second conjugate is converted in situ to the following structure:

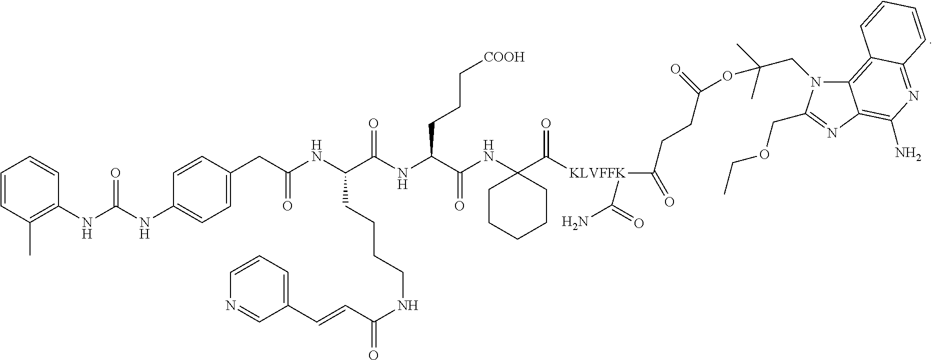

The ratio of the first conjugate to the second conjugate of the nanocarriers of the present invention can be any suitable ratio known by one of skill in the art. In some embodiments, the ratio of the first conjugate to the second conjugate is about 25:1 to 1:25. In some embodiments, the ratio of the first conjugate to the second conjugate is about 25:1 to 1:10. In some embodiments, the ratio of the first conjugate to the second conjugate is about 10:1 to about 1:10. In some embodiments, the ratio of the first conjugate to the second conjugate is about 10:1, 8:1, 5:1, 3:1, or 1:1. In some embodiments, the ratio of the first conjugate to the second conjugate is about 1:1.

### V. Nanofibrils

In some embodiments, the present invention provides a method of forming nanofibrils, comprising contacting a nanocarrier of the present invention with a cell surface or acellular component at a tumor microenvironment, wherein the nanocarrier undergoes in situ transformation to form fibrillary structures, thereby forming the nanofibrils.

When the nanocarrier of the present invention binds with the cell surface or acellular component at a tumor microenvironment, it can undergo an in situ transformation to form nanofibrils, which can disrupt the cells and/or the tumor microenvironment. Transformation of the nanocarrier occurs when the hydrophilic targeting ligands of the nanocarriers bind to the cell surface or acellular component of interest, triggering formation of fibrillary structures which form the nanofibrils.

The tumor microenvironment comprises tumor cells and the surrounding environment, including, but is not limited to, the extracellular matrix, infiltrating host cells, secreted factors, signaling molecules, immune cells, stromal cells, dendritic cells, T cells, myeloid derived suppressor cells, vasculature, blood cells, cytokines, chemokines, growth factors, fibroblast and macrophages, any of which the nanocarrier of the present invention can interact with to form nanofibrils.

Nanocarriers of the present invention can form highly ordered beta-sheet fibrillary structures of the nanofibrils. Without being bound by any particular theory, one possible explanation for forming the beta-sheet fibrillary structures is that the beta-sheet forming peptides in the conjugates influence formation of the beta-sheet fibrillary structures of the nanofibrils.

Nanofibrils of the present invention can have any suitable diameter known by one of skill in the art. In some embodiments, the diameter of the nanofibril is 5 to 50 nm. In some embodiments, the diameter of the nanofibril of the nanofibril is 5 to 30 nm. In some embodiments, the diameter of the nanofibril is 5 to 15 nm. In some embodiments, the diameter of the nanofibril is 5 to 10 nm. In some embodiments, the diameter of the nanofibril is about 5 nm, 6 nm, 7 nm, 8 nm, 9 nm, 10 nm, 11 nm, or about 12 nm.

Transformation of the nanocarrier to the nanofibril can be determined by imaging techniques known by one of skill in the art and by measuring the particle size of the nanocarrier. For example, transformation of the nanocarrier to nanofibril can be determined using TEM imaging wherein the round nanocarrier shapes are transformed into nanofibril structures following binding of the nanocarrier to the cell surface or acellular component at a tumor microenvironment. In another example, nanocarrier size can be determined using dynamic light scattering (DLS). In DLS studies, when the nanocarrier is transformed into nanofibrils, the peak around the diameter of a nanocarrier, for example 10-100 nm, will decrease over time, as the peak around 500 nm-1000 nm increase over time, indicating formation of the nanofibrils.

### VI. Method of Treatment and Imaging

In some embodiments, the present invention provides a method of treating a disease, comprising administering to a subject in need thereof, a therapeutically effective amount of a nanocarrier of the present invention, wherein the nanocarrier forms nanofibrils in situ after binding to a cell surface or acellular component at the tumor microenvironment, thereby treating the disease.

Binding to the cell surface or acellular component can be determined by one of ordinary skill in the art using fluorescence microscopy. Binding to the cell surface or acellular component can be determined when the nanocarrier comprises conjugates with a fluorescent dye as the hydrophobic moiety and the cell is labeled with any fluorescent dye known by one of skill in the art. One of skill in the art can select a suitable dye to use based on which fluorescent dye is used as the hydrophobic moiety. For example, when the nanocarrier comprises conjugates wherein the hydrophobic moiety comprises bis-pyrenes, which is a green fluorescent dye, then the cell can be labeled with a non-green fluorescent dye, such as, but not limited to, a red fluorescent dye or a blue fluorescent dye. In another example, if the hydrophobic moiety comprises a red fluorescent dye, such as, but not limited to, porphyrin, then one of skill in the art can chose a non-red fluorescent dye, such as a green fluorescent dye or blue fluorescent dye.

The tumor microenvironment comprises tumor cells and the surrounding environment, including, but not limited to, the extracellular matrix, infiltrating host cells, secreted factors, signaling molecules, immune cells, stromal cells, dendritic cells, T cells, myeloid derived suppressor cells, vasculature, blood cells, cytokines, chemokines, growth factors, fibroblast and macrophages. Tumor growth and progression can be influenced by interactions of the cancer cells with the microenvironment, which can result in eradication of cancer cells, metastasize of cancer cells, or establishing dormant micrometastases cancer cells. The tumor microenvironment can be targeted for therapeutic responses.

Binding to the acellular component at the tumor microenvironment includes, but is not limited to, binding to the proteins within the extracellular matrix and other ligands, compounds, or dendritic cells which are directly attached to the tumor cell or surrounding cells.

The nanocarriers of the present invention can be administered to a subject for treatment, of diseases including cancer such as, but not limited to: carcinomas, gliomas, mesotheliomas, melanomas, lymphomas, leukemias, adenocarcinomas, breast cancer, ovarian cancer, cervical cancer, glioblastoma, leukemia, lymphoma, prostate cancer, and Burkitt's lymphoma, head and neck cancer, colon cancer, colorectal cancer, non-small cell lung cancer, small cell lung cancer, cancer of the esophagus, stomach cancer, pancreatic cancer, hepatobiliary cancer, cancer of the gallbladder, cancer of the small intestine, rectal cancer, kidney cancer, bladder cancer, prostate cancer, penile cancer, urethral cancer, testicular cancer, cervical cancer, vaginal cancer, uterine cancer, ovarian cancer, thyroid cancer, parathyroid cancer, adrenal cancer, pancreatic endocrine cancer, carcinoid cancer, bone cancer, skin cancer, retinoblastomas, multiple myelomas, Hodgkin's lymphoma, and non-Hodgkin's lymphoma (see, CANCER: PRINCIPLES AND PRACTICE (DeVita, V. T. et al. eds 2008) for additional cancers).

Other diseases that can be treated by the nanocarriers of the present invention include: (1) inflammatory or allergic diseases such as systemic anaphylaxis or hypersensitivity responses, drug allergies, insect sting allergies; inflammatory bowel diseases, such as Crohn's disease, ulcerative colitis, ileitis and enteritis; vaginitis; psoriasis and inflammatory dermatoses such as dermatitis, eczema, atopic dermatitis, allergic contact dermatitis, urticaria; vasculitis; spondyloarthropathies; scleroderma; respiratory allergic diseases such as asthma, allergic rhinitis, hypersensitivity lung diseases, and the like, (2) autoimmune diseases, such as arthritis (rheumatoid and psoriatic), osteoarthritis, multiple sclerosis, systemic lupus erythematosus, diabetes mellitus, glomerulonephritis, and the like, (3) graft rejection (including allograft rejection and graft-v-host disease), and (4) other diseases in which undesired inflammatory responses are to be inhibited (e.g., atherosclerosis, myositis, neurological conditions such as stroke and closed-head injuries, neurodegenerative diseases, Alzheimer's disease, encephalitis, meningitis, osteoporosis, gout, hepatitis, nephritis, sepsis, sarcoidosis, conjunctivitis, otitis, chronic obstructive pulmonary disease, sinusitis and Behcet's syndrome).

In some embodiments, the disease is cancer. In some embodiments, the disease is selected from the group consisting of bladder cancer, brain cancer, breast cancer, cervical cancer, cholangiocarcinoma, colorectal cancer, esophageal cancer, gall bladder cancer, gastric cancer, glioblastoma, intestinal cancer, head and neck cancer, leukemia, liver cancer, lung cancer, melanoma, myeloma, ovarian cancer, pancreatic cancer and uterine cancer. In some embodiments, the disease is selected from the group consisting of bladder cancer, breast cancer, colorectal cancer, esophageal cancer, glioblastoma, head and neck cancer, leukemia, lung cancer, myeloma, ovarian cancer, and pancreatic cancer.

In some embodiments, the nanocarrier of the present invention can be used for combination therapy. In some embodiments, the combination therapy includes a nanocarrier of the present invention and at least one checkpoint inhibitor. Representative checkpoint inhibitors include, but are not limited to, anti-CTLA-4 therapy, an anti-PD-1 therapy, or an anti-PD-L1 therapy, for example. Examples include ipilimumab, nivolumab, pembrolizumab, pidilizumab, atezolizumab, Ipilimumab, and/or tremelimumab, and may include combination therapies, such as nivolumab+ipilimumab.

In some embodiments, the present invention provides a method of imaging, comprising administering to a subject to be imaged, an effective amount of a nanocarrier of the present invention.

Suitable imaging agents for the nanocarriers of the present invention are described above. For example imaging agents include, but are not limited to, paramagnetic agents, optical probes, and radionuclides. Opitcal probes include, but are not limited to fluorescent dyes such as cyanine dyes, bis-pyrenes, and porphyrin.

### VII. Examples

### Example 1: Nanocarriers of BP-FFVLK-YCDGFYACYMDV

This example describes design and synthesis of a smart supramolecular peptide, BP-FFVLK-YCDGFYACYMDV, capable of (1) assembling into nanoparticles (NPs) under aqueous condition and in blood circulation, and (2) in situ transformation into nanofibrillar (NFs) structure upon binding to the cell surface HER2 at the tumour sites. This transformable peptide monomer (TPM), a supramolecular material, was comprised of three discrete functional domains: (1) the bis-pyrene (BP) moiety with aggregation induced emission (AIE) property for fluorescence reporting, and as a hydrophobic core to induce the formation of micellar NPs, (2) the KLVFF β-sheet forming peptide domain originated from β-amyloid (Aβ) peptide, and (3) the YCDGFYACYMDV disulfide cyclic peptide HER2-binding domain, an anti-HER2/neu antibody peptidic mimic derived from the primary sequence of the CDR-H3 loop of the anti-HER2 rhumAb 4D5. Under aqueous condition, the supramolecular peptide would self-assemble into spherical NPs, in which BP and KLVFF domains constituted the hydrophobic core and YCDGFYACYMDV peptide constituted the negatively charged hydrophilic corona. NPs, injected intravenously (i.v.) into mice bearing HER2+ tumours, were found to be preferentially accumulated at the tumour site. Upon interaction with HER2 displayed on the tumour cell surface, the NPs would undergo in situ transformation into fibrillar structural network, with long retention time. Such HER2 binding extracellular fibrillar network was found to greatly suppress the dimerization of HER2 and prevent downstream cell signaling and expression of proliferation and survival genes in the nucleus. These structural transformation-based supramolecular peptides represent a novel class of receptor-mediated targeted therapeutics against cancers.

### Materials and Methods

The preparation of transformable peptide monomers (TPMS) 1′-4′. The hydrophobic bis-pyrene unit (BP-COOH) was synthetized as previously reported (Qiao, S.-L. et al. Thermo-Controlled in Situ Phase Transition of Polymer-Peptides on Cell Surfaces for High-Performance Proliferative Inhibition. ACS Appl. Mater. Interfaces 8, 17016-17022 (2016)). The TPMs 1′-4′ were synthesized by standard solid phase peptide synthesis techniques. The BP-COOH as a hydrophobic part was linked to TPMs 1′-4′ chain. For TPMs 3′ and 4′, PEG₁₀₀₀ as a hydrophilic unit was linked to the peptide to replace HER2 ligand of molecules 1 and 2. The molecular structures of BP dye and peptides were confirmed by matrix-assisted laser desorption ionization time-off light mass spectrometry (ESI and MALDI-TOF mass spectra, Bruker Daltonics).

Self-assembly preparation and characterization of NPs. The TPMs 1′-4′ were dissolved in DMSO to form a solution, respectively. Peptide solution (5 μL) was further diluted with DMSO (995, 795, 595, 395, 195, 95, 15, 0 μL) and mixed with deionized water (0, 200, 400, 600, 800, 900, 980, 995 μL), respectively. The UV-vis absorption and fluorescence spectra (Thermo Scientific, Waltham, Mass.) of different water content mixture solution were measured to validate the formation of NPs. The fresh NPs (99% water content, 20 μM) were used for the measurement as an initial state. The morphology transformation of NPs to NFs was administrated by adding HER2 extracellular receptor protein (expressed in HEK 293 cells, Sigma-Aldrich) and cultured for several hours at 37° C. At different time point (0.5, 6 and 24 h), the solution was used for size/zeta potential (Microtrac, America), CD (JASCO Inc, Easton, Md., USA), and TEM measurement (Philips CM-120 TEM, America). The TEM sample was dyed by uranyl acetate.

Stability of NPs1 in human plasma. The stability of NPs1 was studied in 10% (v/v) plasma from healthy human volunteers. The mixture was incubated at physiological body temperature (37° C.) followed by size measurements at predetermined time intervals up to 168 h.

MCF-7/C6 cells induction process. The induction method of MCF-7/C6 cells was obtained from Professor Jian Jian Li's lab (Departments of Radiation Oncology, University of California Davis). The MCF-7/C6 radioresistant cell line was survived from 25 fractionated ionizing radiations with a total dose of 50 Gy γ rays (2 Gy per fraction, five times per week).

CLSM and SEM validation of NPs structural transformation on cell surfaces. The cells were cultured in glass bottom dishes for 12 h. NPs1-4 (50 μM) was incubated with cells in DMEM at 37° C. for 0.5, 6 and 24 h, respectively. For confocal laser scanning microscope (CLSM, Zeiss LSM710, Jena, Germany) imaging, the specimens were solidified with glutaraldehyde (4%) for 10 min, washed with PBS for 3 times and examined with a 40× or 63× immersion objective lens using a 405 nm laser. To further validate the binding of NPs1 to HER2, rabbit anti-HER2 (29D8) monoclonal antibody (MAb) (Sigma Aldrich, USA) was used to detect the extracellular domain of HER2 on the surface of MCF-7/C6 cells. For SEM (Philips XL30 TMP, FEI Company, Hillsboro), the cells were solidified with glutaraldehyde (4%) overnight and then coated with gold for 2 min.

In vitro cytotoxic assay. MCF-7/C6, MCF-7, SKBR-3 and BT474 cells were used to evaluate the cytotoxicity of NPs1-4. Cells per well were seeded in the 96-well plates (n=3) cultured with DMEM supplemented with 10% FBS and 1% penicillin at 37° C. in a humidified environment containing 5% CO₂. DMSO solution of 1-4 were diluted by DMEM (1.5, 7.5, 15, 75, 150, 300 μM) and then added into each well to incubate with cells. After 48 h of incubation, MTS reagent was added into each well. The relative cell viabilities were measured by Micro-plate reader (SpectraMax M2). Percentage of cell viability represented drug effect, and 100% means all cells survived. Cell viability was calculated using the following equation: Cell viability (%)=(OD490 nm of treatment/OD490 nm of blank control)×100%.

Western blot analysis. MCF-7/C6 cells were treated by different conditions and then collected by centrifugation at 14,000 rpm for 10 min and lysed with a 1% (v/v) Triton X-100 containing lysis buffer (50 mM Tris-HCl, pH 8.0, 150 mM NaCl) with protease inhibitor. Total cellular proteins were estimated using a BCA kit (Applygen). Each sample (50 μg of protein) was subjected to SDS-PAGE and transferred to nitrocellulose membranes. After blocking for 2 h at room temperature with 5% (wt/v) nonfat dry milk in blotto solution (20 mM Tris-HCl, pH 7.5, 150 mM NaCl and 0.1% Tween 20), the membranes were incubated with primary antibody overnight at 4° C. Then the membranes were washed (3×5 min) with TBST solution and incubated with second antibodies for 2 h at room temperature. Signals were visualized by chemiluminescence on a Typhoon Trio Variable Mode Imager. Band density was calculated using NIH Image J software.

For HER2 dimer western blot analysis, MCF-7/C6 cells were treated with indicated protocols and then lysed in buffer containing 137 mM NaCl, 2.7 mM KCl, 10 mM Na₂HPO₄, 1.8 mM KH₂PO₄, 1% Triton X-100 and protease inhibitors cocktail (Sigma-Aldrich). The lysis supernatant was collected after centrifugation at 12,000 rpm for 15 minutes. 0.2% glutaraldehyde was added to the lysis supernatant for 10 minutes at 37° C. The lysis was collected for western blot analysis.

Animal model. All animal experiments were in accordance with protocols No. 19724, which was approved by the Animal Use and Care Administrative Advisory Committee at the University of California, Davis. Female BALB/c nude mice were 6-8 weeks of age (weight 22±2 g), which were purchased from Harlan (Livermore, Calif., USA). MCF-7/C6 cells (5×106 cells per mouse) were inoculated subcutaneously into the flank of each female BALB/c nude mice, respectively. After around 10 days, NPs1-4 (8 mg/Kg) were injected via the tail vein and ex vivo images of tumour, heart, liver, spleen, lung, kidney, intestine, muscle, skin were collected at 10, 24, 48, 72, 168 h post injection. The images were collected by in vivo fluorescence imaging system (Carestream In-Vivo Imaging System FXPRO, USA). Tumour and Main organs (heart, liver, spleen, lung, kidney and brain) were collected and solidified with glutaraldehyde (4%) at 72 h post injection of NPs for TEM imaging.

In vivo therapeutic effect. BALB/c nude mice with MCF-7/C6 cells (5×10⁶ cells per mouse) tumours inoculated subcutaneously into the flank were used in our experiments. The mice were randomly divided into five groups at 10 days post-tumour inoculation. Each of them treated with PBS, NPs1, NPs2, NPs3 and NPs4 every 48 h via i.v. administration. During the process of the treatment (40 days), the tumour volumes and body weight were measured twice per week. In parallel, the therapeutic effect of NPs1 was verified in the mice bearing SKBR-3 and BT474 tumours with similar experimental method mentioned above. For Haematoxylin and eosin (H&E) staining test and Ki-67 test, MCF-7/C6 tumour-bearing mice were sacrificed after three times treatment and tumour tissues were collected.

Statistical analysis. Data are presented as the mean±standard deviation (SD). The comparison between groups was analyzed with the student's t-test (two-tailed). One-way analysis of variance (ANOVA) was used for multiple-group analysis. The level of significance was defined at *p<0.05, **p<0.01, and ***p<0.001. All statistical tests were two-sided.

### Results and Discussion

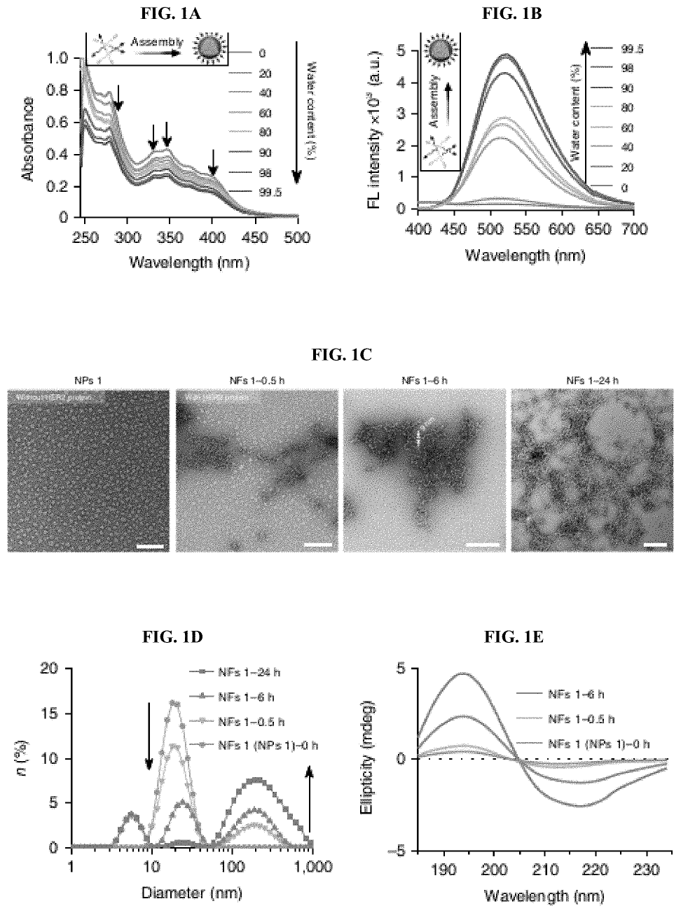

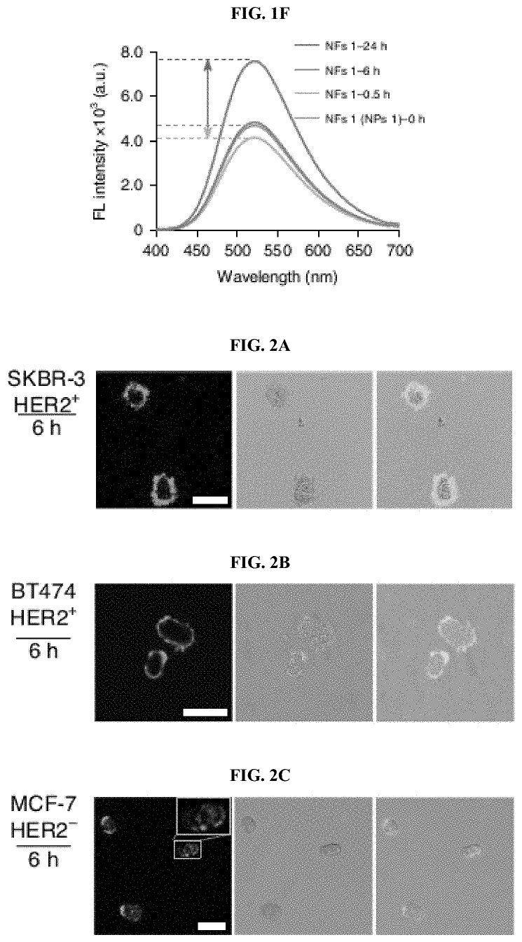

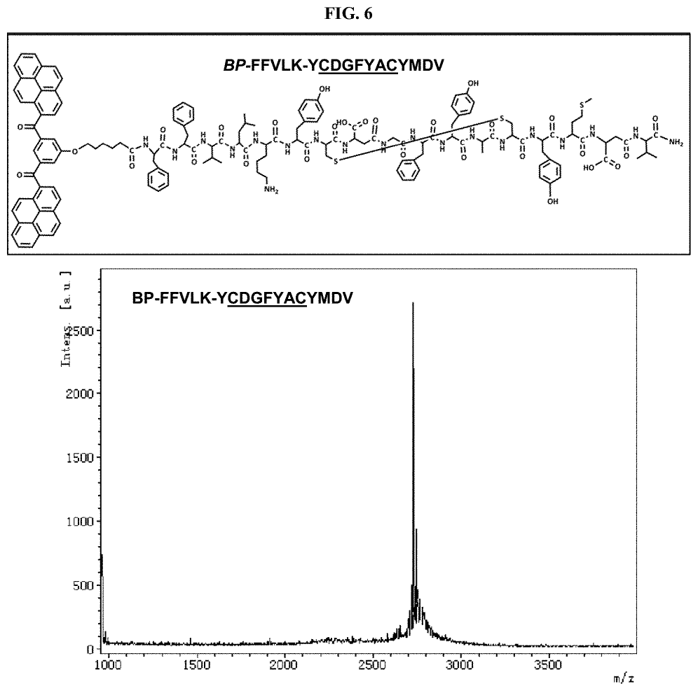

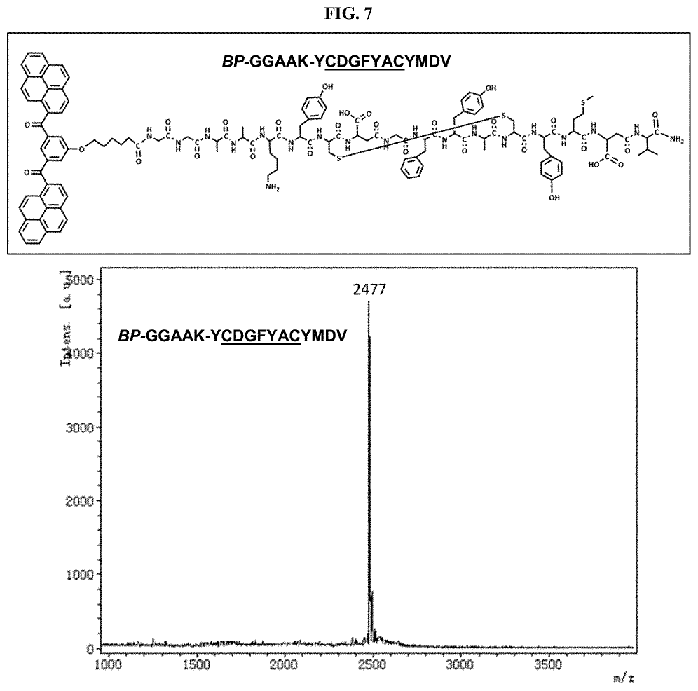

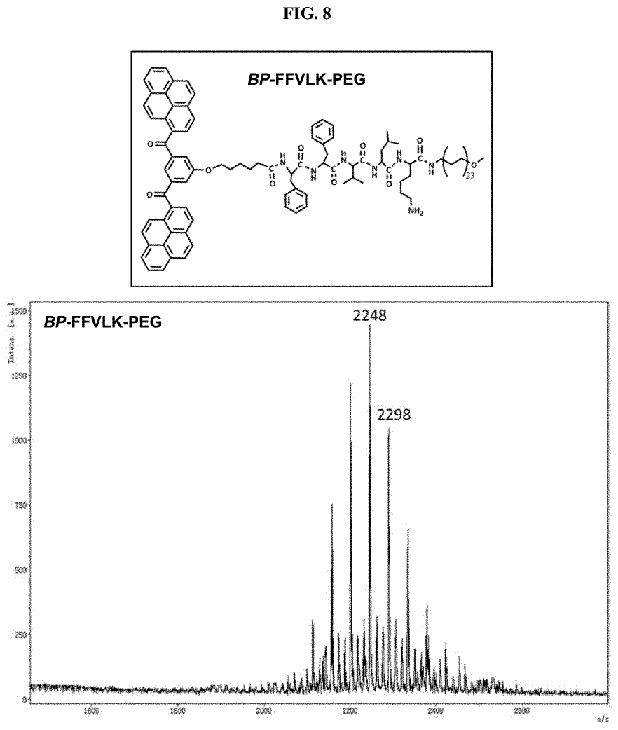

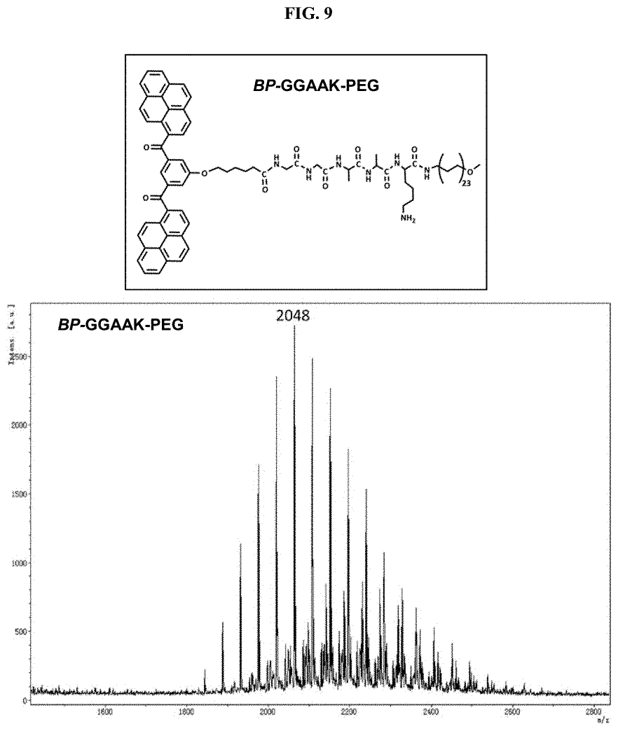

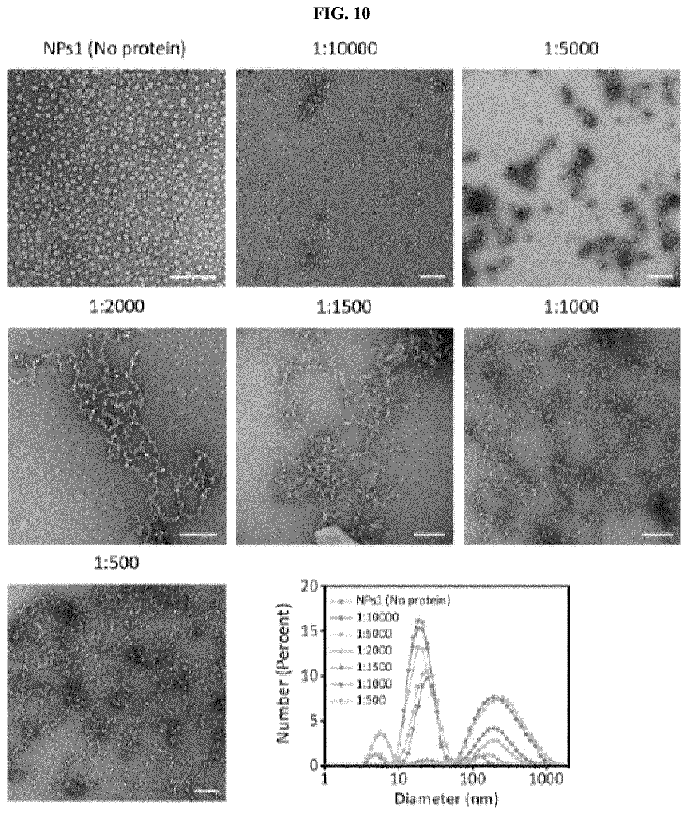

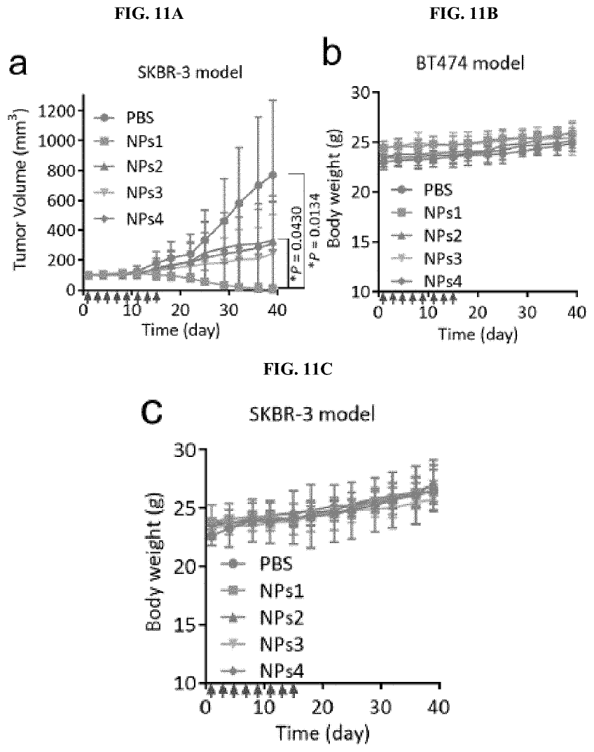

Self-assembly and fibrillar-transformation of supramolecular material. The transformable peptide monomer 1 (TPM1′), BP-FFVLK-YCDGFYACYMDV, was prepared with standard solid-phase peptide synthesis techniques followed by N-terminal capping with bis-pyrene, and its identity was confirmed by MALDI-TOF-MS (FIG. 6). For the purpose of comparisons, TPM2′ (BP-GGAAK-YCDGFYACYMDV), TPM3′ (BP-FFVLK-PEG₁₀₀₀) and TPM4′ (BP-GGAAK-PEG₁₀₀₀) were synthesized as negative controls (Table land FIG. 7-9). As the proportion of water in the mixed solvent (water and DMSO) of TPM1′ solution was increased, there was a gradual decrease in absorption peaks (250-450 nm), reflecting the gradual formation of nanoparticles NPs1 via self-assembly, caused by π-π interaction and strong hydrophobicity of BP and β-sheet forming peptide sequence (FIG. 1A). Concomitantly, the fluorescence peak at 520 nm was found to increase dramatically, due to the AIE fluorescence properties of BP dye (FIG. 1B). TPM2′, TPM3′ and TPM4′ all showed similar self-assembling property. Nanoparticles (NPs1, NPs2, NPs3, and NPs4), assembled from the four TPMs by rapid aqueous dilution method, were analyzed by dynamic light scattering (DLS) and transmission electron microscopy (TEM) (FIG. 1C). The diameters of NPs1-4 were found to be around 20 nm, 30 nm, 25-60 nm and 20 nm, respectively.

TABLE 1
Molecular composition of transformable peptide monomers (TPMs) 1′-4′

| TPM | BP | FFVLK | GGAAK | YCDGFYACYMDV | PEG₁₀₀₀ |
|---|---|---|---|---|---|
| 1′ | + | + | − | + | − |
| 2′ | + | − | + | + | − |
| 3′ | + | + | − | − | + |
| 4′ | + | − | + | − | + |

TPM1′ BP-FFVLK-Y CDGFYACYMDV (with HER2 binding peptide, but without β-sheet forming peptide);
TPM2′ BP-GGAAK-YCDGFYACYMDV (with HERZ binding peptide, but without β-sheet forming peptide);
TPM3′ BP-FFVLK-PEG1000 (without HER2 binding peptide, but with β-sheet forming peptide);
TPM4′ BP-GGAAK-PEG1000 (without HER2 binding peptide nor β-sheet forming peptide).

To investigate the interaction of NPs1 with HER2 in vitro, soluble extracellular domain of HER2 protein as the transformation inducer was chosen. As shown by the TEM images in FIG. 1C, NPs1 was found to maintain a spherical structure at around 20 nm before interaction with HER2. After incubation at room temperature with HER2 protein for only 30 min (molar ratio of HER2 peptide/HER2 protein≈1000:1), a small number of particulate nanofibrillar structures (NFs1, width diameter about 10 nm) became apparent; more NFs1 were detected at 6 h. By 24 h, a fibrillar network with a broad size distribution was clearly detected, indicating that the transformation process was receptor-mediated and time-dependent. No transformation was observed in the NPs1 preparation without the addition of HER2 protein, even after 24 h. The structural transformation from NPs1 to NFs1 was also confirmed in solution by DLS (FIG. 1D), with the gradual decrease in the 20 nm peak and corresponding increase in the 100 to 1000 nm peak over time. In contrast, similar treatment of NPs2, NPs3 and NPs4 solutions with HER2 did not reveal any significant changes over 24 h. Common features of the TPMs that formed these three negative control NPs were the lack of concurrent presence of the two essential domains for receptor-mediated transformation in NPs1: HER2 ligand and KLVFF β-sheet forming peptide. Circular dichroism (CD) spectroscopy was used to monitor the conformation and secondary structure of TPM1′ upon transformation (FIG. 1E). In the initial stage of rapid self-assembly to form NPs1, no obvious secondary structure was observed, probably because hydrophobic interactions induced by BP was too fast to form any intermolecular hydrogen bonds. As NPs1 began to transform to NFs1 over the 24 h period in the presence of HER2, the negative CD signal at 216 nm and positive CD signal at 195 nm progressed gradually over time, indicating (3-sheet formation via hydrogen bonding. In addition to CD, the unique AIE fluorescent property of BP to monitor the kinetics of TPM1′ transformation was exploited. As shown in FIG. 1F, the fluorescence intensity of BP in NPs1 dropped about 10% 30 min after addition of HER2, but turned around and increased as transformation to NFs1 progressed, and eventually reached about 50% increase by 24 h. One plausible explanation for this interesting observation is that the packing density of BP or TPM1′ in the fibrillar networks (NFs1 at 24 h) was significantly higher than that in the initial spherical structure (NPs1). However, during the initial transformation process when the spherical NPs1 were exposed to HER2, there was a transient relaxation in the packing density prior to re-organization into the more densely packed nano-fibrillar network. It is also demonstrated that the particle size of NPs1 in PBS without HER2 remained unchanged over 7 d at 37° C., whether 10% fetal bovine serum (FBS) was present or not.

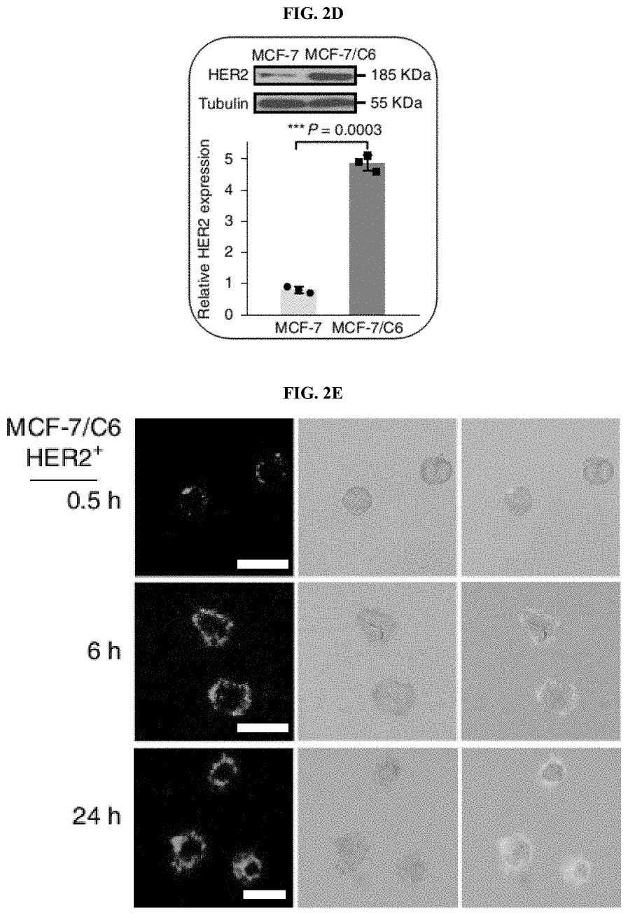

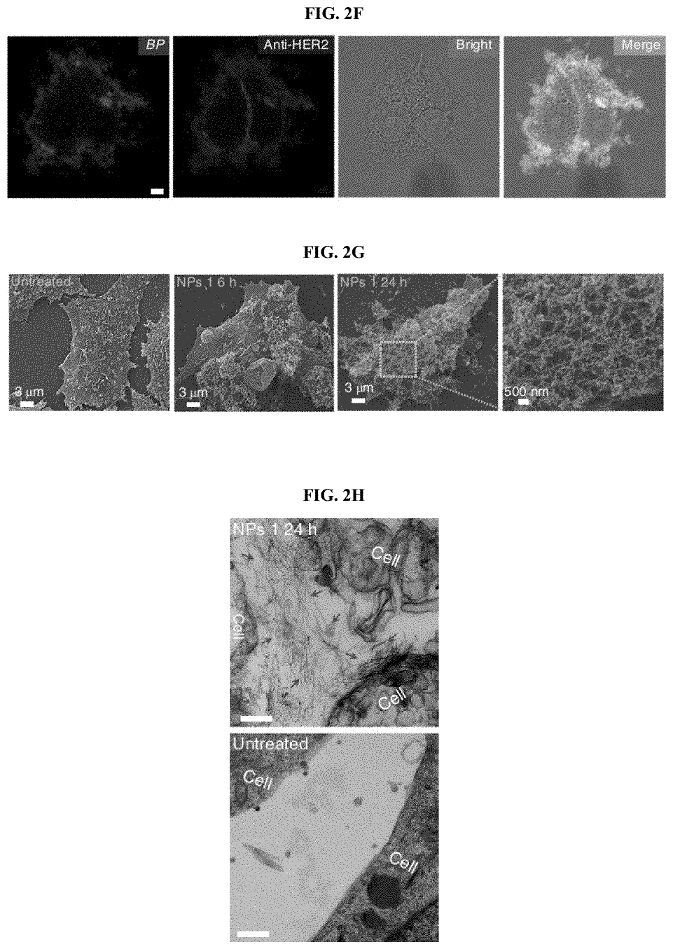

The morphological characterizations of fibrillar-transformation of NPs. To further characterize the interactions between the transforming peptides and cell surface receptors on living cells, HER2+ breast cancer cell lines (SKBR-3 and BT474 cells) were incubated with NPs1, and then used confocal laser scanning microscopy (CLSM) to track the fluorescent green signal emitted by BP (FIG. 2A-2B). After 6 h incubation of NPs1 with these two cell lines, green fluorescence signal was observed on the cell surface rather than inside the cells. In contrast, for MCF-7 breast cancer cells with low-expression level of HER2, the majority of the fluorescent signal was found to reside inside the cells after 6-24 h (FIG. 2C), indicating that cell surface display of HER2 protein was required for transformation of NPs1 to nanofibrillar network at the cell vicinity.

Radiotherapy is commonly used for the management of breast cancer patients. It has previously been reported that long-term fraction ionizing radiation (FIR) can induce HER2 expression, both clinically and in experimental models. In fact, the HER2+ MCF-7/C6 tumour cell line used was derived from HER2 negative human breast cancer MCF-7 cell line that had undergone 30 days of FIR induction, followed by colony formation and clonal isolation. MCF-7/C6 cells exhibit the characteristic of radiation resistance, high expression level of HER2, more aggressive phenotype, and enhanced levels of cancer stem cell properties. The relative expression level of HER2 protein, determined by Western blot, was found to be 5 times higher in MCF-7/C6 cells than in MCF-7 cells (FIG. 2D). After 30 min incubation of MCF-7/C6 cells with NPs1 (100 μM), green fluorescent dots were observed on the cell membrane (FIG. 2E). By 24 h, a luxuriant green fluorescent layer was found surrounding the entire cell.

To further validate the binding of NPs1 to HER2, rabbit anti-HER2 (29D8) monoclonal antibody (MAb) was used to detect the extracellular domain of HER2 on the surface of MCF-7/C6 cells. Anti-HER2 MAb was labeled fluorescent red by the secondary Ab. The NPs1 and the transformed nanofibrillar network (NFs1) were labeled fluorescent green by the intrinsic optical property of BP. As shown in FIG. 2F, green fluorescence overlapped completely with red fluorescence around the periphery of the two cells. The merge image showed overlapping green and red (to form yellow) around the cell surface, except the adhesion interface between the two cells, which was stained by just the anti-HER2 MAb (red fluorescence) and not by the NPs1. This data was consistent with our notion that transformation of NPs1 to NFs1 was triggered by its interaction with cell surface HER2 receptor exposed to the culture medium. The cellular distribution of negative control NPs (NPs2, NPs3 and NPs4) was also investigated in MCF-7/C6 cells. After 24 h incubation, the majority of the fluorescent signals were found inside the cells instead of on the cell surface. Scanning electron microscopy (SEM) confirmed the presence of nanofibrillar network (NFs1) on the surface of NPs1-treated MCF-7/C6 cells but not untreated cells (FIG. 2G). In contrast, no nanofibrillar structure was detected on the surface of cells treated with NPs2, NPs3 or NPs4. Transmission electron microscopy (TEM) were used to better define the ultra-structure of the nanofibrillar network. Similar to the result obtained by SEM, abundant bundles of nanofibrils were detected on the surface of and in between MCF-7/C6 cells after incubation with NPs1 for 24 h. No nanofibrillar structure was detected on untreated MCF-7/C6 cells or cells treated with the three negative control NPs for 24 h. In another negative control experiment in which MCF-7, a cell line with low level of HER2 expression, was incubated for 24 h with NPs1, only minimal amount of nanofibrils were detected on the cell membrane.

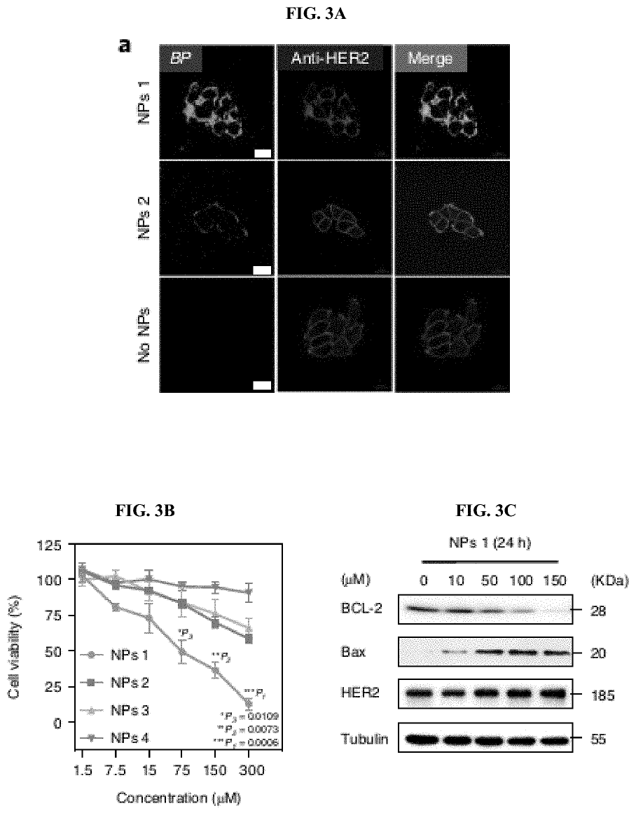

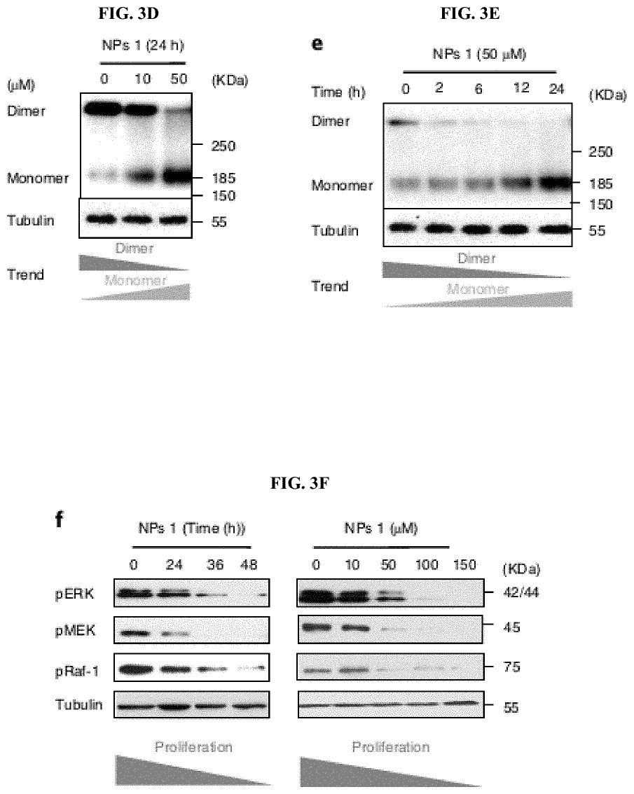

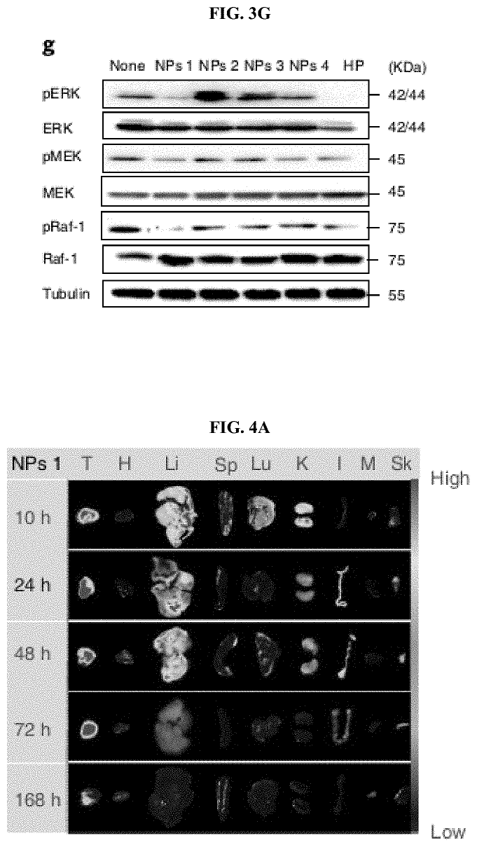

The extracellular and intracellular mechanisms of fibrillar-transformation. It is conceivable that HER2-mediated transformation of nanoparticle (NPs1) to nanofibrillar network (NFs1) could impair HER2 dimerization leading to suppression of downstream signal transduction. To demonstrate this plausible mechanism, MCF-7/C6 cells were incubated with NPs1, NPs2, or PBS for 8 h (FIG. 3A). For the NPs1 treated cells, most of the green fluorescence signal (BP) was found to co-localize with the red fluorescence (anti-HER2), indicating that the nanofibrillar network was closely associated with HER2 receptors displayed on the cell surface. For the cells treated with NPs2, in which the HER2 ligand was present but β-sheet forming peptide was mutated, cell surface green fluorescence was weak. Furthermore, the green/red fluorescent signals on the membrane of the NPs1 treated cells appeared to be significantly thicker and discontinuous, suggesting clustering of nanofibrillar structures and perhaps even disruption of cell membranes.

The cytotoxic effect of NPs1 and the three negative control NPs on MCF-7/C6 cells after 48 h incubation was determined by a MTS assay. As shown in FIG. 3B, treatment with NPs1 resulted in significant cell death at a dose dependent manner, with cell viability of 37% and 13% at 150 μM and 300 μM, respectively. Similar results were obtained for two other HER2+ breast cancer cell lines, SKBR-3 and BT474. However, when MCF-7 cells with low level of HER2 expression were treated by these four NPs, no obvious cytotoxicity was observed even at the highest concentration of 300 μM. This is consistent with our notion that nanotransformation and therefore cytotoxicity of NPs1 is HER2-mediated. To explore the mechanism by which NPs1 induced apoptosis, the expression levels of various pro-apoptotic and anti-apoptotic proteins were evaluated by Western blot. As shown in FIG. 3C, treatment of MCF-7/C6 cells with NPs1 resulted in down-regulation of anti-apoptotic protein Bcl-2 and up-regulation of apoptotic protein Bax, in a dose dependent manner. To study the effect of NPs1 on HER2 dimerization, a simple method of brief chemical crosslinking with 0.2% glutaraldehyde followed by Western blot analysis with anti-HER2 antibody was employed. This method has allowed us to differentiate dimeric HER2 from its monomeric form. It was clear from FIG. 3D and FIG. 3E that NPs1 was able to inhibit HER2 dimerization in a dose-dependent manner. Time course study indicated that NPs1 (50 μM), not only could inhibit HER2 dimerization, it could also promote conversion of HER2 from dimeric form to monomeric form. The effect of NPs1 on MAPK pathway was also studied by Western blot. A significant decrease in pErk, pMek and pRaf-1 level over time was observed when the cells were treated with 50 μM of NPs1; this inhibitory effect was dose-dependent (FIG. 3F). For the purpose of comparison, MCF-7/C6 cells were incubated with 50 μM of each NPs for 36 h, and Herceptin was used as a positive control (FIG. 3G). Like Herceptin, NPs1 was able to strongly inhibit phosphorylation of Erk, Mek and Raf-1. In contrast, the three negative control NPs did not significantly alter the phosphorylation level of Erk, Mek and Raf-1. Together, these data strongly support transformation of NPs1 to nanofibrillar network on the surface of HER2+ tumour cells causes inhibition of HER2 dimerization and conversion of HER2 dimers to monomers, leading to inhibition of downstream proliferation and survival cell signaling, and cell death.

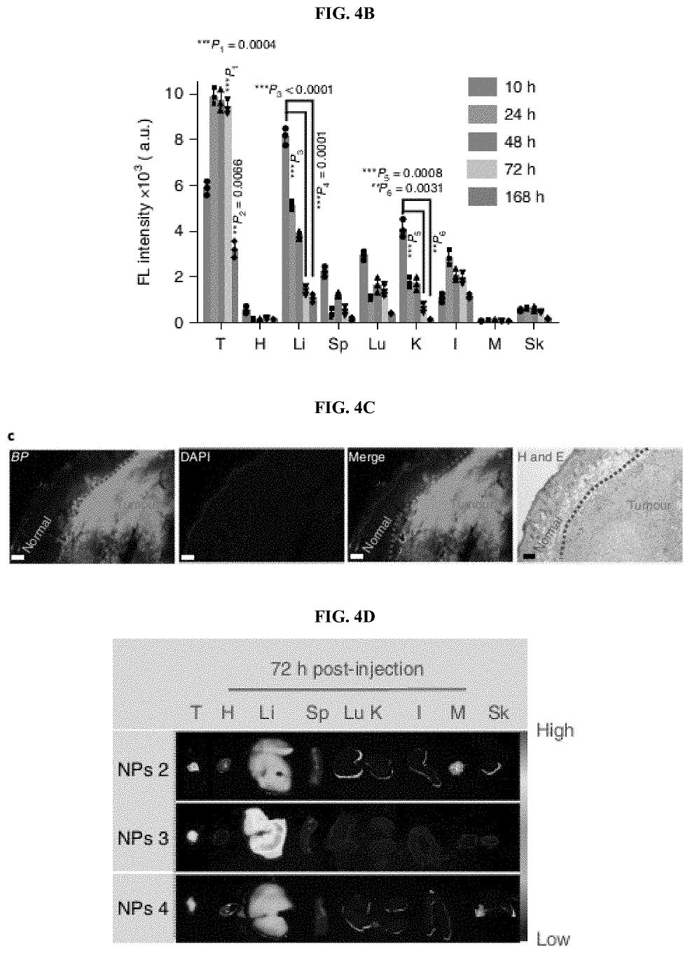

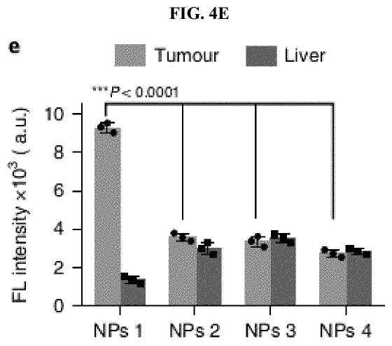

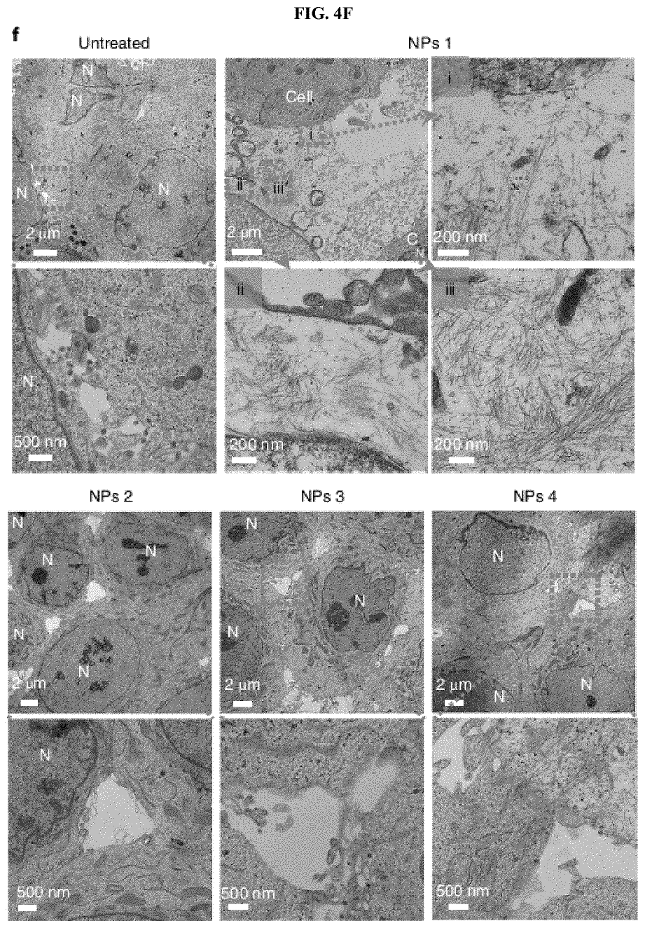

In vivo evaluation of fibrillar-transformation. NPs1 was found to be non-toxic; blood counts, platelets, total protein, creatinine and liver function tests obtained from normal Balb/c mice treated with 8 consecutive q.o.d. doses of NPs1 were within normal limit. For biodistribution studies, mice bearing MCF-7/C6 tumour were given i.v. NPs1; 10, 24, 48, 72 and 168 h later, main organs were collected for ex vivo fluorescent imaging study (FIG. 4A-4B). Fluorescent uptake by tumour and normal organs such as liver, lung and kidneys were high at 10 h. Fluorescent signal persisted in tumour for over 3 days, with significant residual signal even after 7 days. In contrast, fluorescent signal in normal organs began to drop after 10 h and was almost undetectable in main organs at 72 h. At 72 h, tumour and overlying skin were excised for fluorescent microscopy studies. It was clear that compared to intense fluorescent signal in tumour, negligible signal was detected in the normal skin (FIG. 4C). Histologic examination of excised normal organs did not reveal any pathology. Similar in vivo biodistribution studies on NPs2, NPs3 and NPs4 were also performed in the same tumour model. At 72 h, fluorescent signal of tumour derived from mice treated with NPs1 was found to be 2-3 times higher than that of mice treated with NPs2-4 (FIG. 4D-4E). Prolonged retention of fluorescent signal in NPs1 treated mice, even after 7 days, could be attributed to in situ receptor-mediated transformation of NPs1 into NFs1 networks at the tumour microenvironment. TEM studies on excised tumour, 72 h after i.v. administration, showed abundant bundles of nanofibrils in the extracellular matrix of tumour sections. No such nanofibrils were observed in the negative control NP-treated and untreated mice (FIG. 4F). In addition, many cells in the tumour excised from NPs1-treated mouse appeared to be dying with large intercellular spaces. The TEM images of other organs (heart, liver, spleen, lung, kidney and brain), excised from the same mouse were found to be normal, without any sign of nanofibrillar networks, which was consistent with the result of the optical imaging and histopathology studies mentioned above.

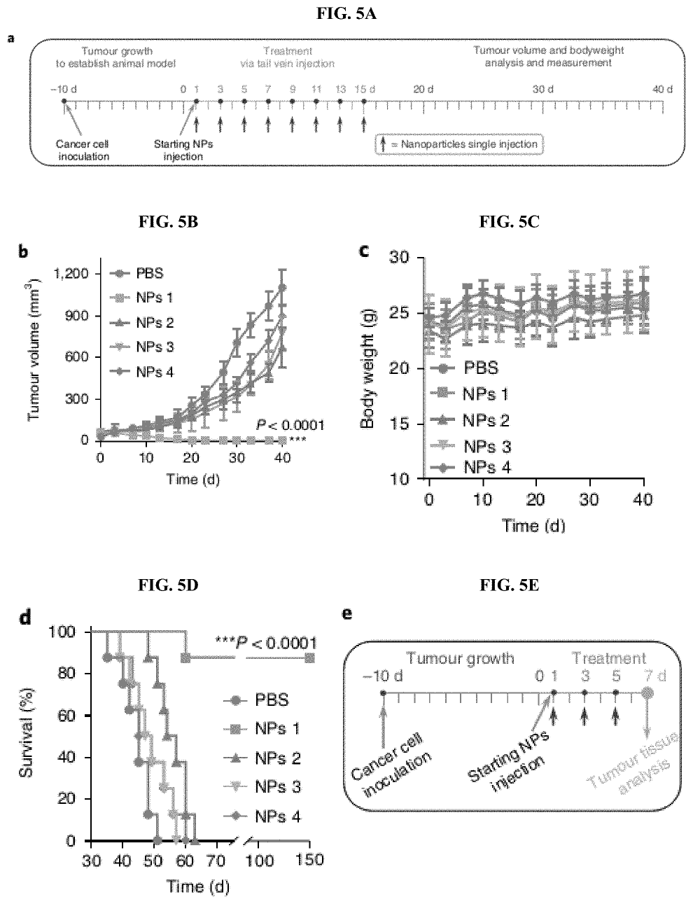

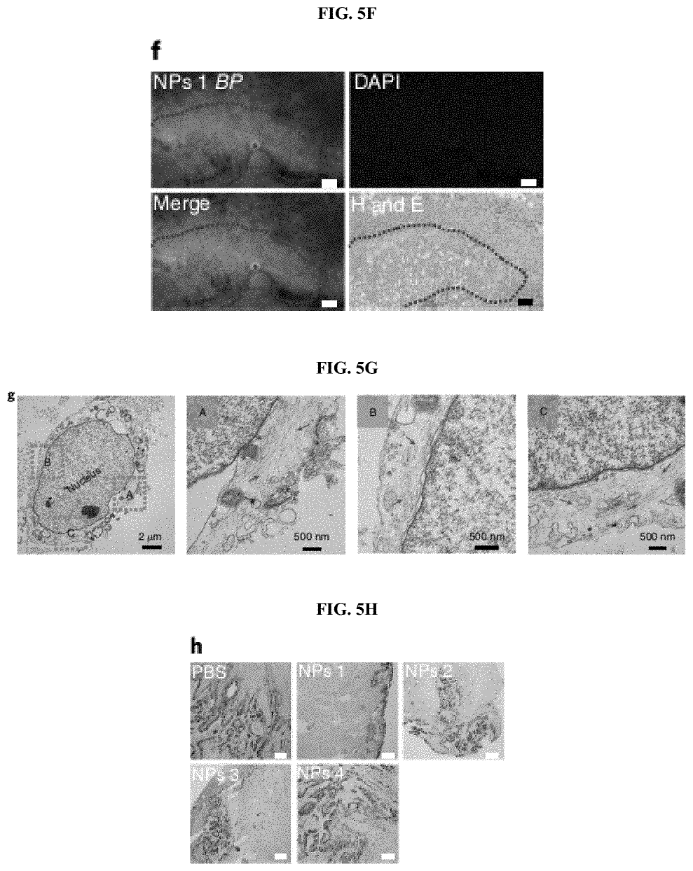

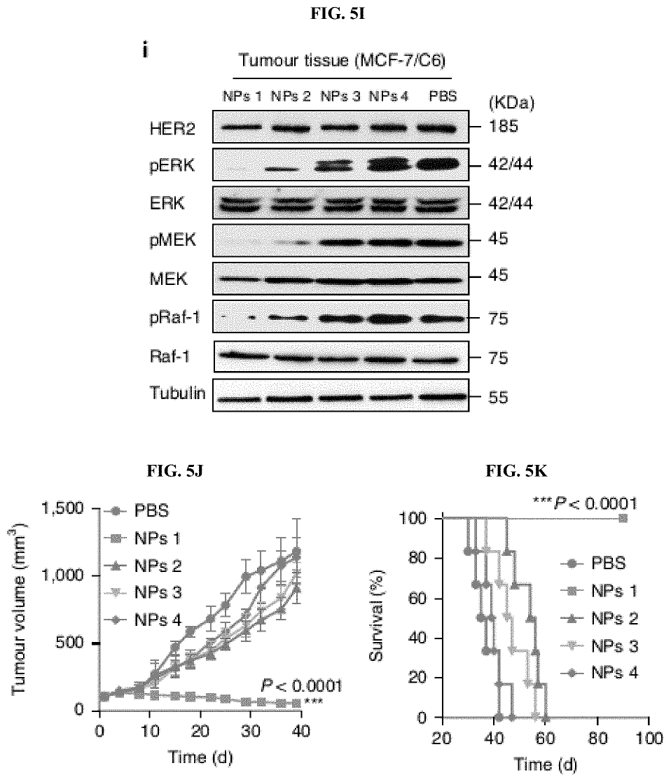

Anti-tumour activity of fibrillar transformable NPs. Therapeutic efficacy studies of NPs1, NPs2, NPs3 and NPs4 were performed in MCF-7/C6 HER2+ breast cancer bearing mice (FIG. 5A). When tumour volume of mice reached about 50-80 mm³, NPs were injected consecutively 8 times q.o.d. (day 1, 3, 5, 7, 9, 11, 13, 15) via tail vein and observed continuously for 40 days. As shown in FIG. 5B, tumour volume of NPs1 treated mice gradually shrunk and was totally eliminated after treatment without any sign of recurrence. In contrast, none of the other 3 negative control groups (NPs2, NPs3, and NPs4) elicited any significant tumour response. None of the mice in this therapeutic study showed any symptoms of dehydration and significant body weight loss during the entire 40 d therapeutic study (FIG. 5C). The survival curves correlated well with tumour growth results (FIG. 5D). Seven of the eight mice receiving NPs1 treatment survived over 150 days without any sign of tumour recurrence. One of these eight mice, no longer with detectable tumour, died at around day 60 for unknown reason. In contrast, all mice in the PBS, NPs2, NPs3 and NPs4 treated groups died within 51, 63, 57, and 60 days respectively. This result is highly encouraging and clearly demonstrates the clinical potential of receptor-mediated transformative supramolecular nanotherapeutics (e.g. NPs1) against solid tumour in general, and more specifically against HER2+ tumours.

To better understand the in vivo anti-tumour mechanism of NPs1, mice were sacrificed and residual tumours collected for biochemical and morphological assessment after 3 consecutive q.o.d injections of NPs1 (FIG. 5E). Frozen sections were obtained for fluorescent microscopy and hematoxylin and eosin (H&E) stain (FIG. 5F). The degree of cell kill was found to correlate well with that of fluorescent intensity; necrosis was detected in the tumour areas with strong fluorescence intensity. To understand how the nanofibrillar network kill the HER2+ tumour cells, high magnification TEM on tumours obtained from NPs1-treated mice was performed. The TEM image of a necrotic or necroptotic cell in FIG. 5G revealed that the plasma membrane was broken, with abundant fibrillar nanostructures present inside the broken cell. Some of the nanofibrillar bundles were found adjacent to the nuclear envelope of the nucleus. No significant cell kill was detected in tumour sections obtained from mice treated with PBS, NPs2, NPs3 or NPs4. Tissue section staining for Ki-67 marker is a good way to assess the anti-proliferative effects of NPs1 in vivo. After 3 treatments with NPs1, the expression level of Ki-67 in tumour tissue was markedly decrease, compared to the tumour obtained from mice treated with negative control NPs (FIG. 5H).

It has been shown above that NPs1 could inhibit HER2 dimerization and phosphorylation of Erk, Mek and Raf-1 in HER2+ cell line in cell culture. Here similar Western blot studies are performed on tumours excised from mice that had undergone 3 consecutives q.o.d. treatments of NPs1. As shown in FIG. 5I, total HER2 level remained unchanged, but phosphorylation of Erk, Mek and Raf-1 was found to be markedly decrease, compared to the other negative control groups. Together, the data clearly demonstrated that receptor-mediated transformative supramolecular nanotherapeutic NPs1 was highly effective in suppressing downstream proliferative and survival cell signaling at the tumour tissue level. To better investigate the universality of NPs1 as an efficacious therapeutic against HER2+ tumours, two other human HER2+ breast cancer xenograft models (SKBR-3 and BT474) were chosen for our studies. As shown in FIG. 5J-5K, the tumour volume of mice treated with NPs1 responded very well with complete elimination of SKBR-3 tumour, and almost completed elimination of BT474 tumour by day 40. In contrast, the tumour volumes of the PBS control groups had grown to 1200-1500 mm³ on day 40.

One known side-effect of Herceptin is cardiotoxicity. It cannot be given to patient together with cardiotoxic drug such as doxorubicin. Thus far, there was no observed cardiotoxic effects in our xenograft studies with NPs1. No uptake of NPs1 in the myocardium was detected. This is not surprising as the coronary vessels are expected to be intact and the 20 nm NPs1 will not be able to reach the myocardium. The fact that NPs1 was highly efficacious against three different HER2+ tumours warrants further preclinical and clinical development of NPs1 against HER2+ breast, ovarian, gastric, and bladder cancers. There is good clinical evidence that some originally HER2 negative breast cancers can be induced to express HER2 after long-term fraction ionizing radiation (FIR). This further expands the patient population who may benefit from this novel receptor-mediated transformable nanotherapy (RMTN).

It has been demonstrated that 8 consecutive q.o.d doses of NPs1 alone as a monotherapy was efficacious in curing a large percentage of mice bearing relatively small (≤100 mm³) HER2+ breast cancer xenografts.

### Example 2: Nanocarriers Comprising a Plurality of Two Different Conjugate

Immune checkpoint blockade (ICB) therapy has revolutionized clinical oncology. One of the main contributing factors for ICB resistance is defects in Teff cell homing to the tumour sites. This example describes a 28 nm non-toxic peptidic micellar nanoparticle, displaying LXY30, an α₃β₁ integrin targeting ligand. Upon interaction with α₃β₁ integrin over-expressed in many epithelial cancers, these nanoparticles would undergo in situ transformation at the tumour microenvironment (TME) into nanofibrillar structural network. The nanofibrillar network not only promotes cytotoxic CD8⁺ T cell homing to and macrophage re-education at the tumour sites, but also allowed sustain release of TLR 7/8 immunoagonist (resiquimod), via esterase at the TME, resulting in elimination of syngeneic 4T1 breast cancer and Lewis lung cancer models in mice, when given together with anti-PD-1 antibody. These structural transformation-based supramolecular peptides represent an innovative class of receptor-mediated targeted immunotherapeutics against cancer via enhancing T cell tumour homing and reprogramming of TME.

This example describes a ligand-receptor-mediated, peptide-based, and non-toxic dual-ligands fibrillar transformable nanoplatform, capable of mounting systemic anti-immune response against cancers. This nanoplatform, initially in nanoparticle form, is self-assembled from two smart transformable peptide monomers TPM1 and TPM2. TPM1, LXY30-KLVFFK(Pa), was comprised of three discrete functional domains: (1) the high-affinity and high-specificity LXY30 cyclic peptide (cdG-Phe(3,5-diF)-G-Hyp-NcR) ligand that targets α₃β₁ integrin heterodimeric transmembrane receptor expressed by many solid tumours, (2) the KLVFF β-sheet forming peptide domain originated from β-amyloid (Aβ) peptide, and (3) the pheophorbide a (Pa) moiety with fluorescence property, serving as a hydrophobic core to induce the formation of micellar nanoparticles. TPM2, proLLP2A-KLVFFK(R848), was also comprised of three discrete functional domains: (1) proLLP2A, the “pro-ligand” version of LLP2A, which is a high-affinity and high-specificity peptidomimetic ligand against activated α₄β₁ integrin of lymphocytes, (2) the same KLVFF β-sheet forming peptide domain, and (3) R848 (resiquimod), a hydrophobic toll-like receptors (TLRs) 7/8 agonist, grafted to TPM2 main chain via an ester-bond. In proLLP2A, the carboxyl group of LLP2A is esterized by 3-methoxy-1-propanol such that it will not interact with normal lymphocytes and mesenchymal stem cells during blood circulation. At the TME with abundant esterase, proLLP2A will be converted to LLP2A to facilitate homing of immune cells to the tumour sites. Similarly, esterase-responsive release of R848 would occur at the TME to activate antigen-presenting cells (APCs), promote immune cells to produce anti-tumour response factors, and reverse the phenotype of macrophage from M2 to M1.

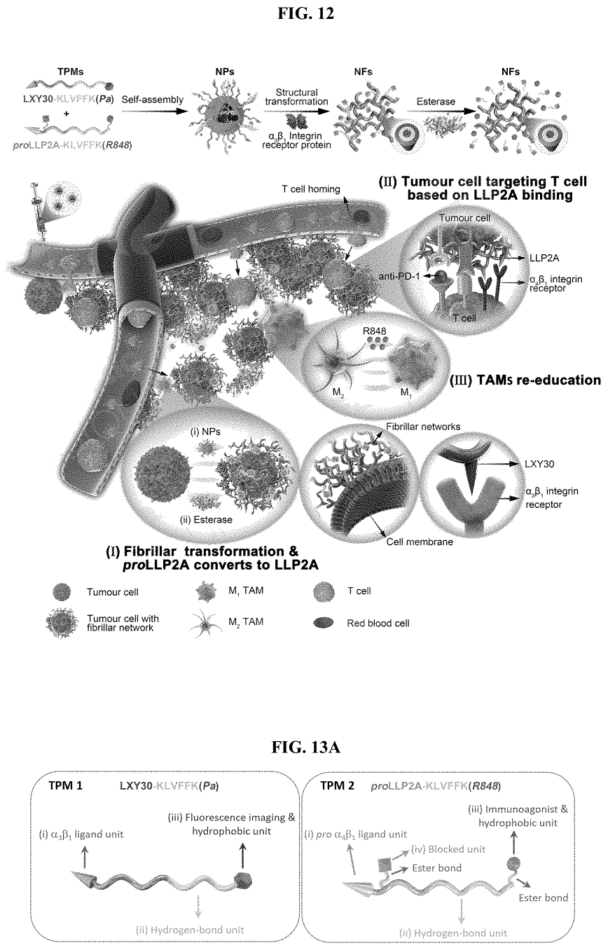

Under aqueous condition and in blood circulation, TPM1 and TPM2 would self-assemble into one spherical transformable nanoparticle (T-NP) at a ratio of 1:1, in which KLVFFK(Pa) and KLVFFK(R848) domains constituted the hydrophobic core, and LXY30 and proLLP2A ligand peptides constituted the hydrophilic corona. Upon interaction with α₃β₁ integrin receptor protein displayed on the tumour cell membrane, the T-NPs would undergo in situ transformation into nanofibrillar (T-NFs) structural network on the surface of tumour cells and within the TME where the tumour associated exosomes were abundant, thus maintaining a prolonged retention of the nanofibrillar network at the tumour sites (at least 7 days). In this case, the more hydrophilic proLLP2A peptide ligand would be displayed on the outer surface of the fibrils, while the hydrophobic Pa and R848 would be sequestered at the core of the fibrils. With the elevated esterase in the TME and on the tumour cells, proLLP2A would quickly be converted to LLP2A (T cell ligand) against activated α₄β₁ integrin. LLP2A displayed on the fibrils would facilitate the homing and retention of activated immune cells such as T_{eff} cells (e.g. CD8⁺ T) cells at the TME and adjacent to the tumour cells. It would also enhance the interaction between T cell receptor (TCR) of Teff and major histocompatibility complex (MHC) of tumour cells. The addition of anti-PD-1 ICB therapy would further enhance the anti-tumour immune response by activating the cytotoxic T cell and reversing the dysfunction and exhaustion of T_{eff}. In addition, the sustained release of R848 from the nanofibrillar network as a result of the elevated esterase at the tumour site would reverse the immunosuppressive TME. These structural transformation-based supramolecular peptides represent an innovative class of receptor-mediated targeted immunotherapeutics against cancer via enhancing T cell homing to the tumours and improving the TME from an immunosuppressive state to a durable immunoactive state (FIG. 12).

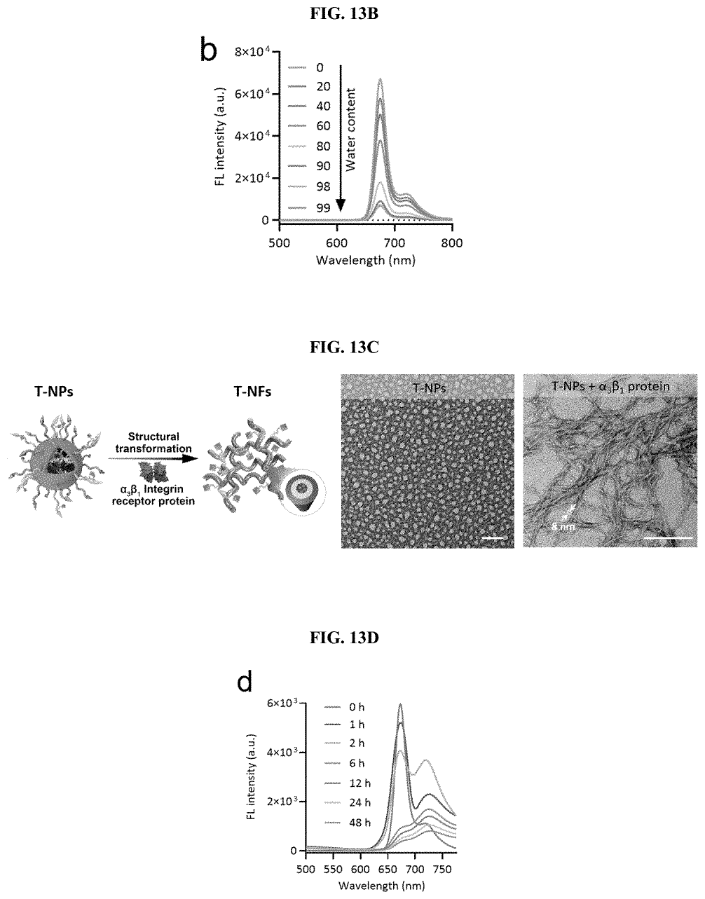

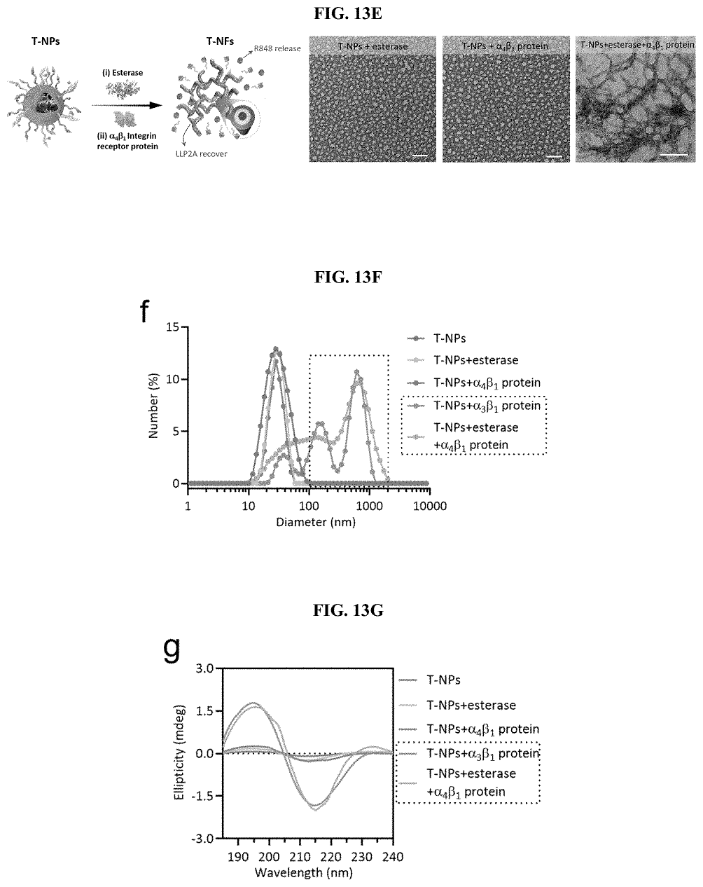

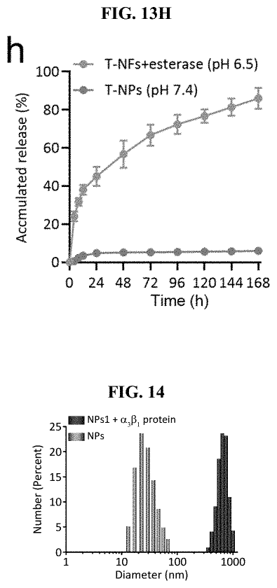

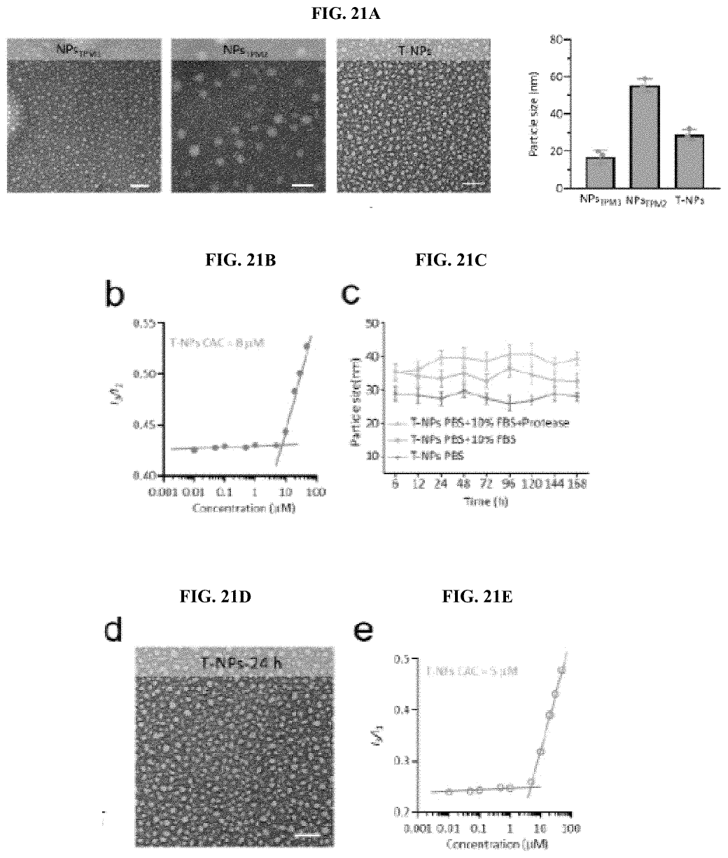

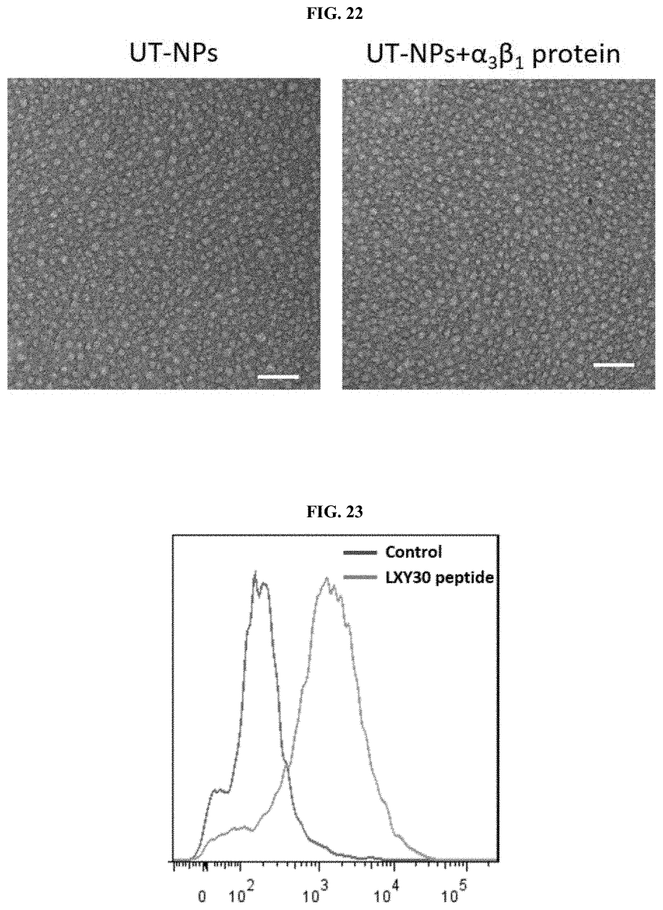

Self-assembly and fibrillar transformation of the nanoplatform. Two transformable peptide monomers (TPM1: LXY30-KLVFFK(Pa); TPM2: proLLP2A-KLVFFK(R848)) were synthesized and characterized (FIG. 13A and FIG. 20). As the proportion of water in the mixed solvent (water and DMSO) of the TPM1 and TPM2 mixture solution (the ratio of 1:1) was increased, there was a gradual decrease in fluorescence peak at 675 nm due to the ACQ properties of Pa dye (FIG. 13B), reflecting the gradual formation of transformable NPs (named as T-NPs) via self-assembly. Concomitantly, there was a modest decrease in the absorption peak at both 405 and 680 nm. Nanoparticles were analyzed by transmission electron microscopy (TEM) and dynamic light scattering (DLS). TPM1 and TPM2 each alone were able to self-assemble to form spherical nanoparticles (NPs_{TPM1} and NPs_{TPM2}) at 18 and 55 nm, respectively. T-NPs, assembled from 1:1 mix of TPM1 and TPM2, yielded a spherical structure at around 28 nm, which fell between the sizes of NPs_{TPM1} and NPs_{TPM2} (FIG. 21A). The critical aggregation concentrations (CAC) of T-NPs was determined to be 8 μM (FIG. 21B). It was also demonstrated that T-NPs could maintain good serum stability and proteolytic stability over 7 days at 37° C. (FIG. 21C).

To verify the receptor-mediated fibrillar transformable process of T-NPs in vitro, soluble α₃β₁ integrin protein (receptor for LXY30) was added to T-NPs solution. After 24 h of incubation at room temperature, a fibrillar network (T-NFs, width diameter about 8 nm) with a broad size distribution was clearly detected (FIGS. 13C, 13F). No transformation was observed in the T-NPs preparation without the addition of α₃β₁ integrin protein, even after 24 h (FIG. 21D). The CAC of T-NFs was determined to be 5 μM, which was lower than that of T-NPs (8 μM), indicating that T-NFs has higher propensity to form nanostructures than T-NPs (FIG. 21E). The fluorescence of Pa was also used to monitor the fibrillar-transformation process of T-NPs (FIG. 13D). Addition of α₃β₁ integrin protein to T-NPs solution resulted in a gradual decrease in the fluorescence intensity of Pa, and a remarkable shift of the fluorescent peak towards the red region from 680 nm to 725 nm within the first 2 h, consistent with the change in aggregation structure of Pa from spherical structure to fibrillar configuration during that time. Responsiveness of proLLP2A and LLP2A displayed on the T-NPs surface to soluble α₄β₁ integrin protein in the presence and absence of esterase was investigated (FIGS. 13E-13F). Soluble α₄β₁ integrin protein alone was not able to alter the spherical structure of T-NPs displaying proLLP2A, even after 24 h of incubation. In contrast, successive addition of esterase, followed by soluble α₄β₁ integrin protein was able to elicit conversion of spherical T-NPs to fibrillar network after 24 h of incubation. This result confirmed that esterase was able to convert pro-ligand proLLP2A to ligand LLP2A, which in turn was able to trigger receptor-mediated transformation of T-NPs to T-NFs. Circular dichroism (CD) spectroscopic analysis of the transformation process of T-NPs showed a gradual progression of a negative signal at 216 nm and a positive signal at 195 nm upon incubation with α₃β₁ integrin protein or combination esterase/α₄β₁ integrin protein, indicative of β-sheet formation (FIG. 2G) and consistent with TEM results shown in FIGS. 13C and 13E. In vitro release behaviour of R848 from T-NFs was studied at pH 6.5 with addition of esterase to simulate TME condition. As shown in FIG. 13H, about 45% of R848 was released the first 24 h, after which the release rate gradually slowed down and about 86% cumulative release was observed by 168 h, indicating that prolonged and sustained release of R848 could occur at the TME. To demonstrate the unique transformable property of T-NPs, a related control untransformable nanoparticle (UT-NP) was formed by assembly of two TPMs without β-sheet forming KLVFF peptide sequence, at a ratio of 1:1 (TPM3: LXY30-KAAGGK(Pa) and TPM4: proLLP2A-KAAGGK(R848)). As expected, α₃β₁ integrin protein was unable to transform UT-NPs to fibrillar structures even after 24 h, indicating that the β-sheet peptide was required for the transformation of T-NPs to T-NFs (FIGS. 20 and 21).

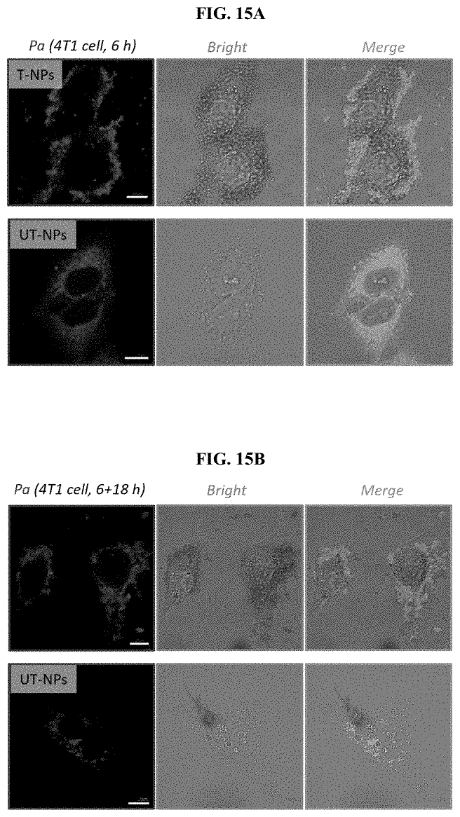

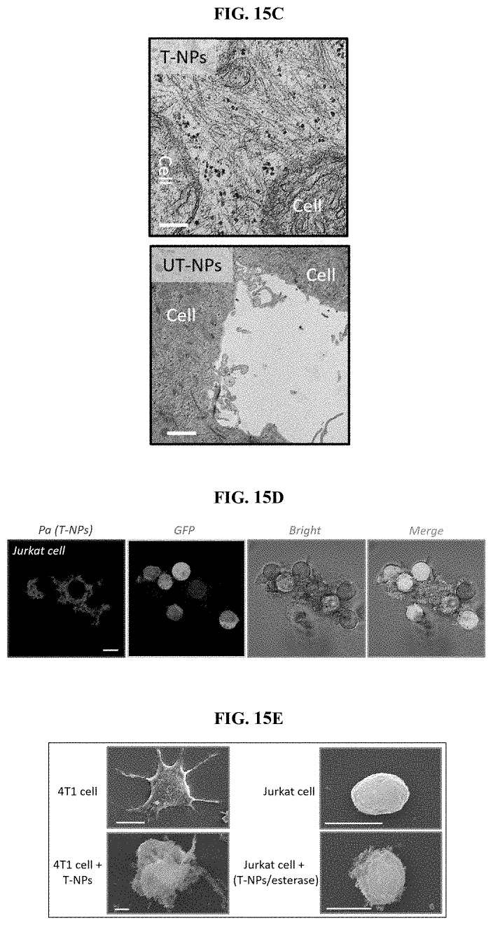

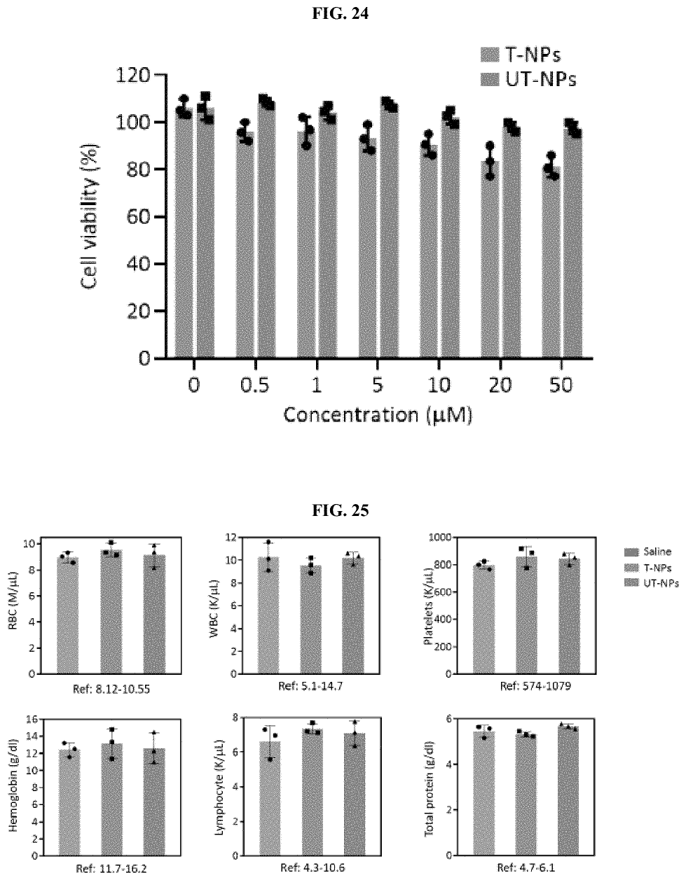

In vitro evaluation of fibrillar-transformation of nanoparticles and T effector cell homing to tumour sites. To further characterize the interaction between transformable nanoparticles and α₃β₁ integrin receptors on the surface of living cells, α₃β₁ integrin expressing 4T1 murine breast cancer cell was chosen. Flow cytometry analysis confirmed that LXY30, the high affinity α₃β₁ integrin ligand, did bind to 4T1 tumour cells (FIG. 23). It was also found that T-NPs was slightly cytotoxic against 4T1 cells, with 85% cell viability at 50 μM (FIG. 24). The distribution of NPs was investigated by tracking the red fluorescent signal emitted by Pa using confocal laser scanning microscopy (CLSM). Six hours after incubation of 4T1 cells with T-NPs, a strong red fluorescence signal was observed on the cell surface and its vicinity but not inside the cells (FIG. 15A). In contrast, the fluorescent signal of Pa in UT-NPs-treated group was found to be concentrated primarily in the cytoplasm of the cells. To study the retention and stability of formed nanofibrillar network on the surface of tumour cells, unbound NPs were washed off after 6 h of incubation and fresh medium without NPs was added to incubate cells for another 18 h. T-NPs treated cells still retained strong red fluorescence signals on the cell surface at 24 h (FIG. 15B). In sharp contrast, only weak fluorescence signal was observed inside the cells treated with UT-NPs after 24 h. This is probably due to the enzymatic degradation of the already endocytosed UT-NPs after 18 h of incubation, but without any new endocytic uptake during that time period. TEM images confirmed the presence of nanofibrillar network (T-NFs) on the surface of, and between 4T1 cells after incubation with T-NPs for 24 h, but absence of such nanofibrillar structures on cells treated with UT-NPs (FIG. 15C). The fibrillar structures further away from the cell surface were probably induced by the secreted tumour exosomes displaying α₃β₁ integrin proteins. The effect of esterase on the interactions between T-NPs and T-cell surface α₄β₁ integrin, after converting pro-ligand pro-LLP2A to LLP2A displayed on the surface of T-NPs was investigated. Live GFP transfected Jurkat T-lymphoid leukemia cells with high expression level of constitutively activated α₄β₁ integrin protein were used to mimic T cells. As shown in FIG. 15D, after 6 h incubation of Jurkat cells with T-NPs (pre-treated with esterase), a luxuriant red fluorescent layer was found surrounding the Jurkat cells, indicating that the conversion of pro-ligand to LLP2A ligand was successful. Scanning electron microscopy (SEM) confirmed the presence of fibrillar network on the surface of T-NPs-treated 4T1 cells and esterase pre-treated T-NPs-treated Jurkat cells (FIG. 15E).

To simulate the processes of initial fibrillar transformation of T-NPs on the 4T1 cells surface followed by T cell homing, first incubated 4T1 cells with T-NPs for 6 h, unbound T-NPs were then washed off, followed by addition of fresh medium containing esterase but without T-NPs. After 1 h of incubation, Jurkat cells were added and incubated with 4T1 cells for 2 or 4 h. After that, unbound Jurkat cells were gently removed prior to CLSM imaging (FIG. 15F). As expected, a fibrillar structure layer with red fluorescence was detected surrounding 4T1 cells surface, and Jurkat cells (GFP+) were found to interact with the red fluorescent fibrillar network and in close proximity to 4T1 breast tumour cells, after 2 h of incubation. As the incubation time was increased to 4 h, many more Jurkat cells were found clustered around the 4T1 tumour cells, which was consistent with our notion that fibrillar network would facilitate the homing of immune cells such as T-cells to the tumour sites. SEM imaging provided critical evidences that the nanofibrillar structures had played a significant role in direct physical contact between 4T1 cells and Jurkat cells through nanofibrillar network (FIG. 15G).

The conversion of TAMs from an immunosuppressive M2-polarized phenotype to an anti-tumorigenic M1-polarized phenotype is one of the major immunotherapeutic strategies for reversing the immunosuppressive tumour microenvironment. Macrophage polarization states demonstrate hallmark morphology, e.g., elongated projections for M2-like cells as opposed to a round and flattened morphology for M1-like counterparts. IL-4 has been used to induce bone marrow derived macrophages (BMDM) to M2-polarized macrophages, as reflected by the increase in expression level of the metabolic checkpoint enzyme arginase-1 (Arg1) and mannose receptor-1 (Mrc1). R848 has been reported to be a powerful driver of the M1-phenotypes in vitro, resulting in elevated level of interleukin 12 (TL-12) and nitric oxide synthase (Nos2) produced by these cells. The possibility of using T-NFs to re-educate macrophages from M2 phenotype to M1 phenotype was investigated. In the nanoplatform, R848 was covalently linked to TMP2 via an ester bond. Therefore, not unexpected, incubation of 4T1 cells with T-NFs, preformed from T-NPs with soluble α₃β₁ integrin protein, did not have significant effect on M2-polarized macrophages induced by IL-4 (FIG. 15H). No significant change in macrophage morphology and expression level of Arg1 and Mrc1 was observed even after 12 h, which can be explained by the lack of R848 released from T-NFs. In contrast, addition of esterase to the culture medium followed by 12 h incubation resulted in morphological change of M2-state macrophages towards M1-state, a decrease in Arg1 and Mrc1, and an increase in IL-12 and Nos2 expression as measured by qPCR. These changes were even more pronounced after 24 h, at which time the macrophages were completely transformed to a round and flattened morphology (M1-like), with further decrease in Arg1 and Mrc1, and increase in IL-12 and Nos2 expression. The ability of T-NFs to anchor at the TME, afforded the sustained release of R848 from the fibrillar network, will generate a durable anti-cancer immunoactive TME.

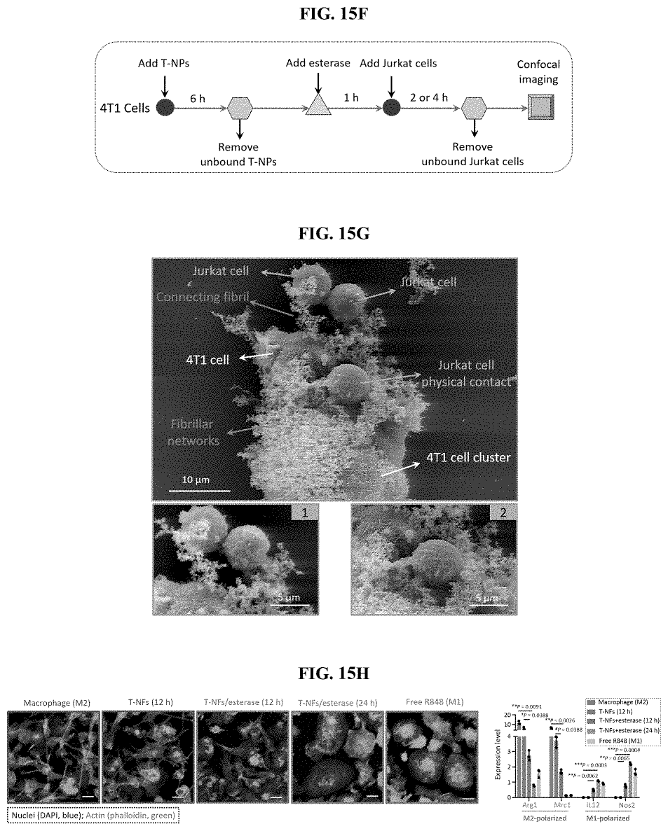

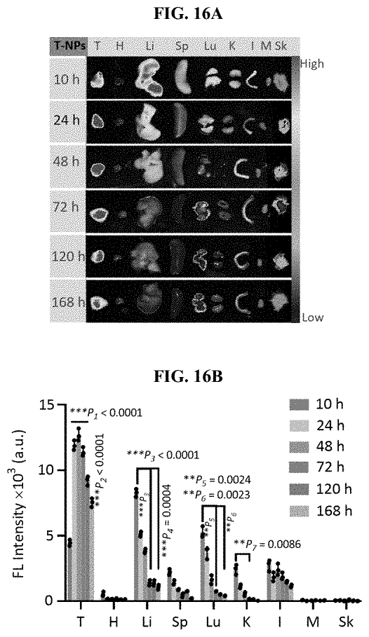

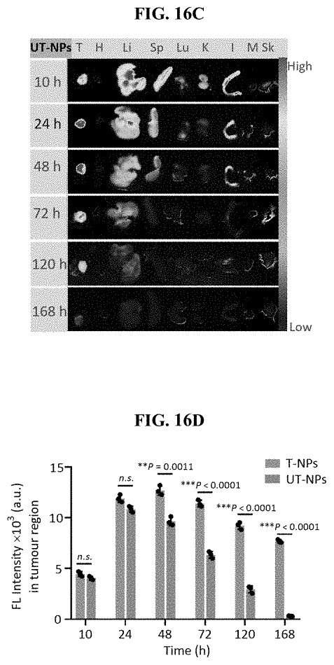

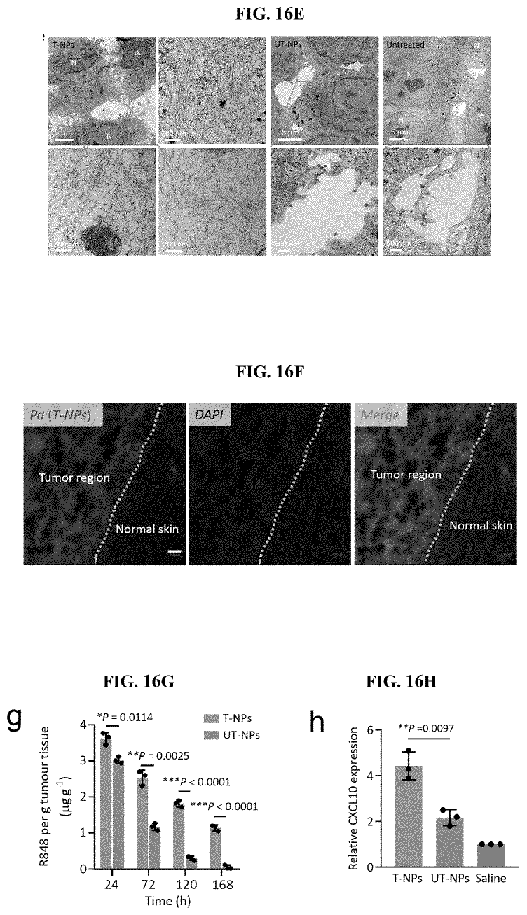

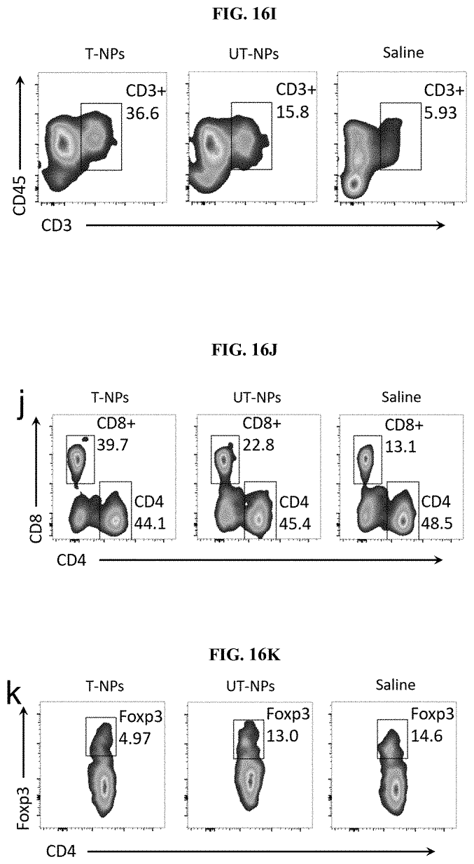

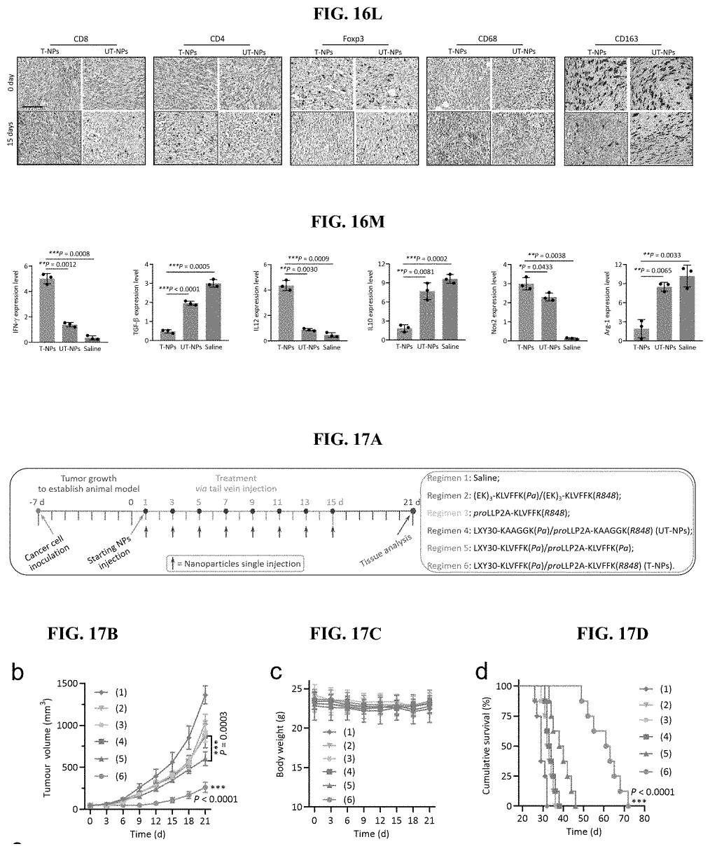

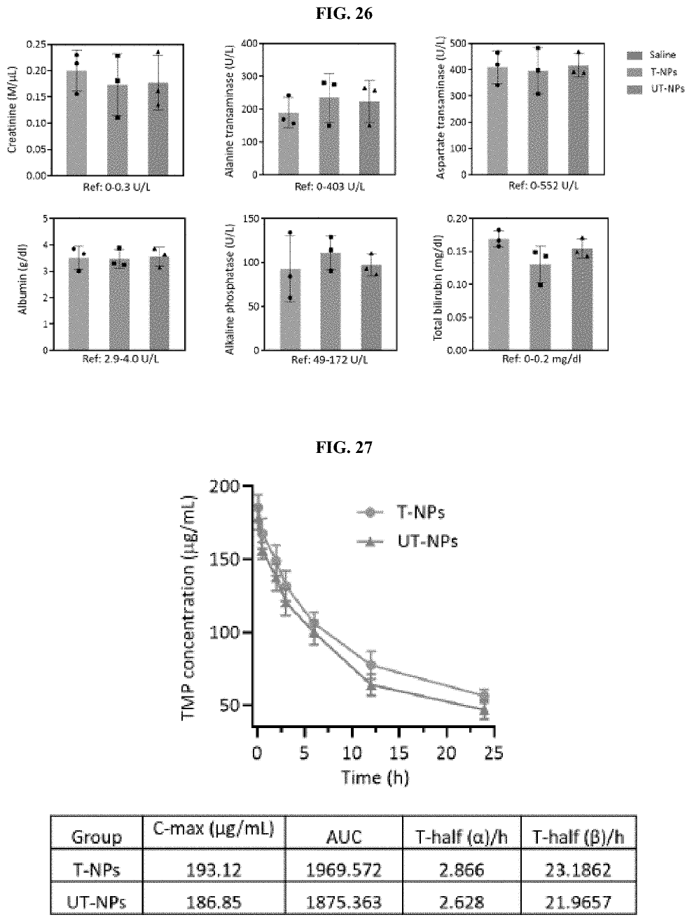

In vivo evaluation of fibrillar-transformation of nanoparticles and tumour homing of T effector cells. T-NPs was found to be non-toxic: blood counts, platelets, creatinine and liver function tests obtained from normal Balb/c mice treated with eight consecutive q.o.d. intravenous (i.v.) doses of T-NPs were within normal limits (FIGS. 25-26). In vivo blood pharmacokinetics (PK) studies indicated that T-NPs possessed a long circulation time (T-half (α): 2.866 h and T-half (β): 23.186 h), indicating its stability during circulation (FIG. 27). For biodistribution studies, T-NPs were tail vein injected into Balb/c mice bearing syngeneic orthotopic 4T1 breast cancer; 10, 24, 48, 72, 120 and 168 h later, tumour and main organs were excised for ex vivo fluorescent imaging (FIG. 16A-16B). Significant fluorescent signal of Pa was found to persist in tumour tissue for over 168 h, while fluorescent signal in normal organs began to decline after 10 h and was almost undetectable in the main organs at 72 h. In sharp contrast, fluorescent signal of Pa at tumour tissue treated by UT-NPs was found to gradually decline over time after peaking at 24 h (FIG. 16C-16D). By 168 h, less than 2.88% of the peak fluorescent signal for UT-NPs remained in the tumour, whereas for T-NPs, over 59.89% signal remained in the tumour (FIG. 16D). Prolonged retention of fluorescent signal in T-NPs-treated mice could be attributed to in situ receptor-mediated transformation of T-NPs into T-NFs networks in the TME. TEM studies on excised tumour sections, 72 h after i.v. administration, showed abundant bundles of nanofibrils in the extracellular matrix while no such nanofibrils were observed in negative control UT-NPs-treated and saline-treated mice (FIG. 16E). Fluorescent micrographs of tumour and overlying skin revealed intense fluorescent signal in tumour region but negligible signal in normal skin. This is consistent with our notion that (1) T-NPs would leak into the TME through leaky tumour vasculatures (EPR effect), followed by interaction with α₃β₁ integrin on tumour cells and tumour associated exosomes to generate T-NFs, and (2) blood vessels are not leaky in normal skin (FIG. 16F). The tissue distribution of R848 over time was also determined with high pressure liquid chromatography-mass spectroscopy (HPLC-MS). It was found that with T-NPs, R848 uptake by tumour was significantly higher than that of other normal organs at 24 h, and that the retention of R848 at tumour site was quite high at 1.18 μg per g tissue even at 7 days after injection (FIG. 16G). Although UT-NPs could also deliver significant amount of R848 to the tumour site (80% of what T-NPs could deliver), but retention of R848 at the tumour site was much lower than that of T-NPs. Prolonged retention of R848 in tumour site indicates that a sustained immune-active TME could be achieved with T-NPs.

To evaluate if the nanofibrillar networks displaying LLP2A and R848 at the TME could promote in vivo T cells homing to the tumour sites, tumours from T-NPs-treated mice were excised on day 15 after a single i.v. injection of T-NPs, and the immune cell populations within the tumours were analyzed by flow cytometry, immunohistochemistry (IHC) and qPCR. Experiment using UT-NPs as an untransformable/endocytic negative control group was also performed at the same time. It was found that tail-vein injection of T-NPs had resulted in a sustained immunoactive TME. First, T-NPs was found to significantly stimulate the production of chemokine CXCL10 at the tumour site (FIG. 16H), which was known to facilitate T effector cells recruitment. It was observed that the proportion of CD45⁺CD3⁺ and CD45⁺CD3⁺CD8⁺ T cells in the T-NPs-treated tumour tissue was substantially higher than those from mice treated with endocytic UT-NPs or saline alone (FIG. 16I-16J). More specifically, the percentage of CD3⁺CD8⁺ T effector cells in tumours was found to be 18 and 4-fold increase, relative to that of saline and UT-NPs-treated mice, respectively (FIG. 16J). Second, it was found that the relative abundance of CD4⁺Foxp3⁺ Tregs at the tumour site was substantially lower in mice that received T-NPs treatment than those in mice treated with UT-NPs, i.e. (4.97% versus 13.0%) or saline (4.97% versus 14.6%) (FIG. 16K). The ratios of tumour-infiltrating CD8⁺ killer T cells to immunosuppressive Tregs (CD3⁺CD4⁺Foxp3⁺), which could be an indicator of anti-tumour immune balance, were found to be the highest in T-NPs treated group (FIG. 16J-16K). IHC staining of tumour tissue sections also confirmed an increase in CD8/CD4 and decrease in Foxp3 (FIG. 16L). Third, IHC staining of tumour sections demonstrated an increase in M1-polarized macrophage marker CD68 and a decrease in M2-polarized macrophage marker CD163 in the T-NPs treated group, compared to the tumour tissue treated by UT-NPs. This could be explained by the sustained release of R848 at the tumour site, causing the phenotypic re-education of TAMs. Fourth, gene expression level of cellular immune related markers (IFN-γ, TGF-β) and macrophage markers (IL-12, IL-10, Nos2 and Arg-1) were also evaluated by qPCR. As shown in FIG. 16M, the high expression level of IFN-γ and low expression level of TGF-β in the tumour tissue confirmed that a strong tumour-specific immune response had been elicited. Furthermore, the secretion of IL-12 and Nos2 was found to be significantly upregulated, while the secretion of IL-10 and Arg-1 was significantly down-regulated, indicating a significant phenotypic conversion of TAMs from M2 state to M1 state, with T-NPs treatment, but not UT-NPs treatment nor saline control.

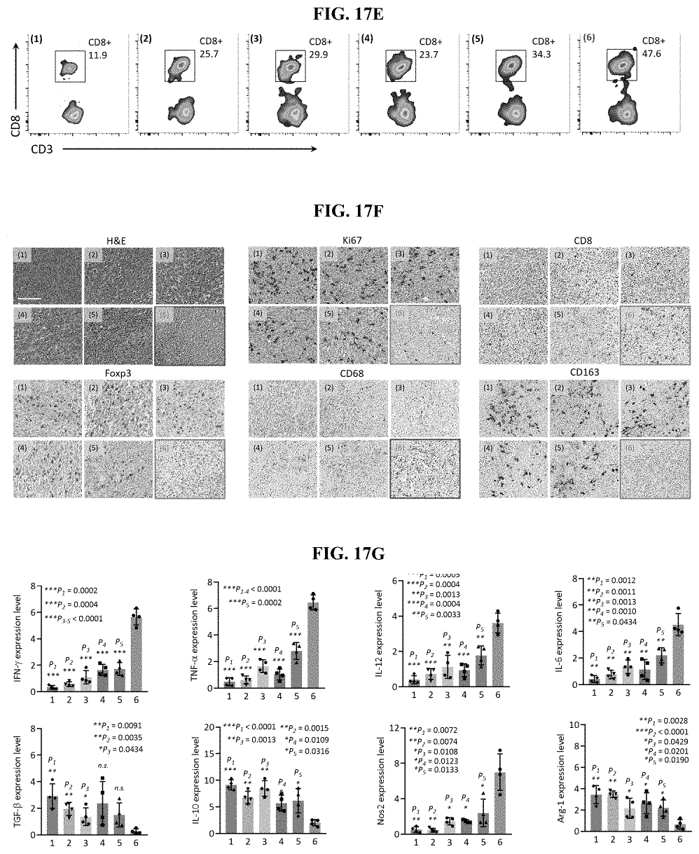

Therapeutic efficacy study was performed in syngeneic orthotopic 4T1 breast cancer-bearing mice. Mice were randomly divided into six groups, each received a different treatment regimen: (1) Saline; (2) (EK)₃-KLVFFK(Pa)/(EK)₃-KLVFFK(R848); (3) proLLP2A-KLVFFK(R848) (single monomer); (4) LXY30-KAAGGK(Pa)/proLLP2A-KAAGGK(R848) (untransformable UT-NPs); (5) LXY30-KLVFFK(Pa)/proLLP2A-KLVFFK(Pa) (fibrillar-transformation but absence of R848); (6) LXY30-KLVFFK(Pa)/proLLP2A-KLVFFK(R848). Regimen 6 is the complete T-NPs, containing all 4 critical components: LXY30, proLLP2A, R848, and KLVFF, whereas regimen 2, 3, 4 or 5 all lack some components of T-NPs. When tumour volume reached about 50 mm³, all treatment regimens were tail vein injected consecutively eight times q.o.d. and the mice were continuously observed for 21 days (FIG. 17A). As shown in FIG. 17B, regimen 2, 3 and 4 were inactive. Regimen 5 (fibrillar-transformation but no R848) demonstrated significant tumour suppression compared to group 2, 3 and 4. Regimen 6 (T-NPs, both fibrillar-transformation and R848) was found to be the most efficacious with significant tumour growth suppression (FIG. 17B) and prolonged survival (FIG. 17D), indicating the importance of combination T cells homing strategy and sustained release of TLR7/8 agonist. None of the mice in this therapeutic study showed any symptoms of dehydration nor significant body weight loss during the entire treatment period (FIG. 17C). The survival curves correlated well with tumour growth results. The mice treated by regimen 6 (or T-NPs) achieved a longer median survival time (62 d) compared with other treatment groups (29, 32.5, 33.5, 33.5 and 39 d for regimen 1, 2, 3, 4, and 5, respectively).

To elucidate the mechanism of immunotherapeutic effects induced by transformable nanoparticles, the tumour tissues were collected and used flow cytometry to quantify tumour-infiltrating CD3⁺ (CD45⁺CD3⁺) and CD8⁺ (CD45⁺CD3⁺CD8⁺) T cells (FIG. 17E). Only the treatment regimen capable of in situ fibrillar transformation and presentation of proLLP2A (regimen 5 and 6) significantly increased the frequency of CD3⁺ and CD8⁺ T cells within the tumours, particularly in combination with immune adjuvant R848 in T-NPs (regimen 6), which was consistent with the observed strongest anti-tumour effects in T-NPs. Tumour sections (H&E) obtained from mice treated with T-NPs revealed a marked decrease in Ki-67 expression, an increase in CD8⁺ T cells, and a decrease in Foxp3 (Treg cells), compared with other control groups (FIG. 17F). There was an increase in CD68 and a decrease in CD163, indicating that the phenotype of macrophages was reversed after 8 doses of T-NPs. It is known that CD8⁺ T cells secrete cytokines IFN-γ and TNF-α to kill tumour cells. The expression levels of IFN-γ and TNF-α in the tumour tissue were further evaluated by qPCR. As shown in FIG. 17G, treatment regimen 6 (T-NPs) was the most efficacious in restoring the immunoactive state of the tumour microenvironment, with the highest expression levels of IFN-γ and TNF-α. In addition, T-NPs also significantly induced expression of IL-12, IL-6 and Nos2, and suppressed expression of TGF-β, IL-10 and Arg-1, leading to the suppression of the Treg cells recruitment and re-education of M2-like macrophages to M1 phenotype.

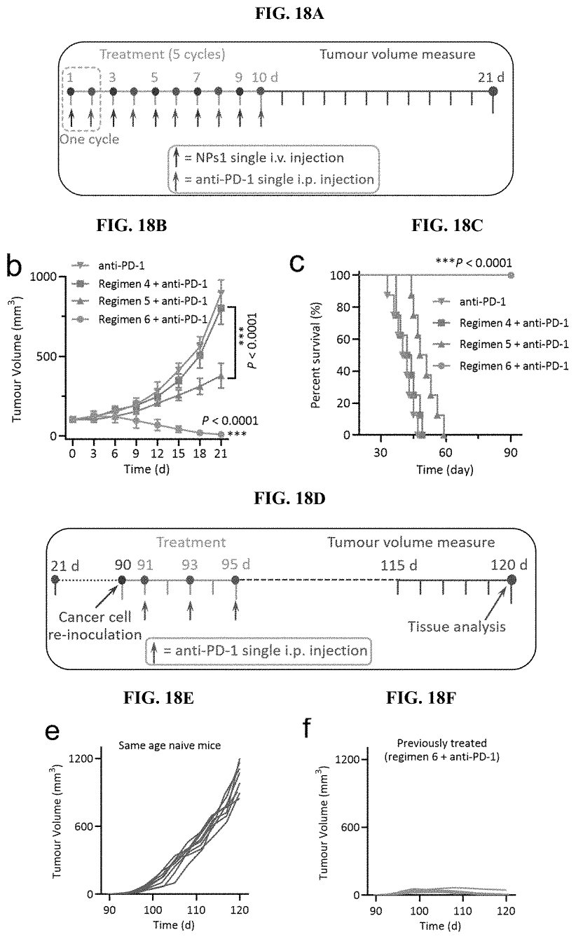

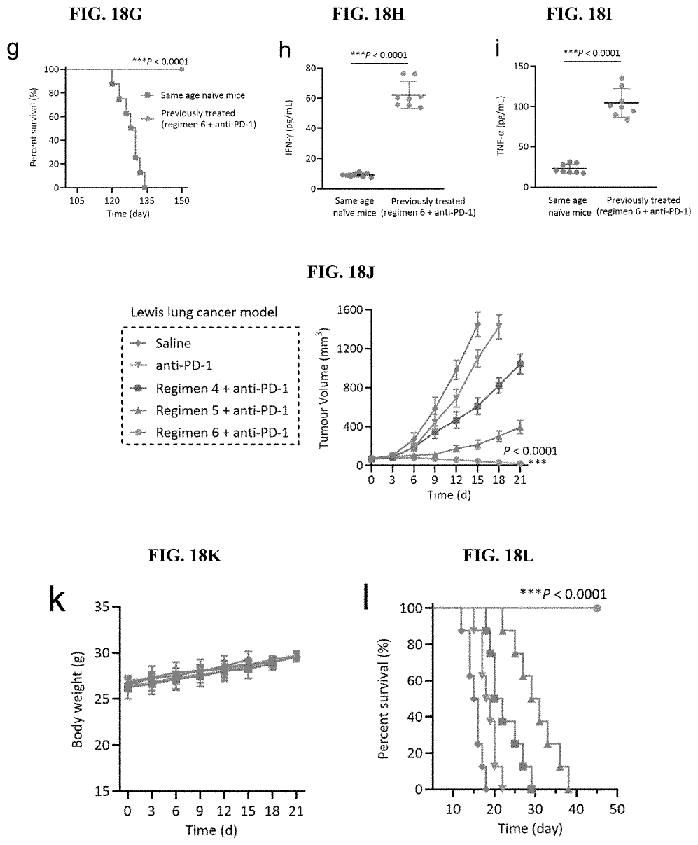

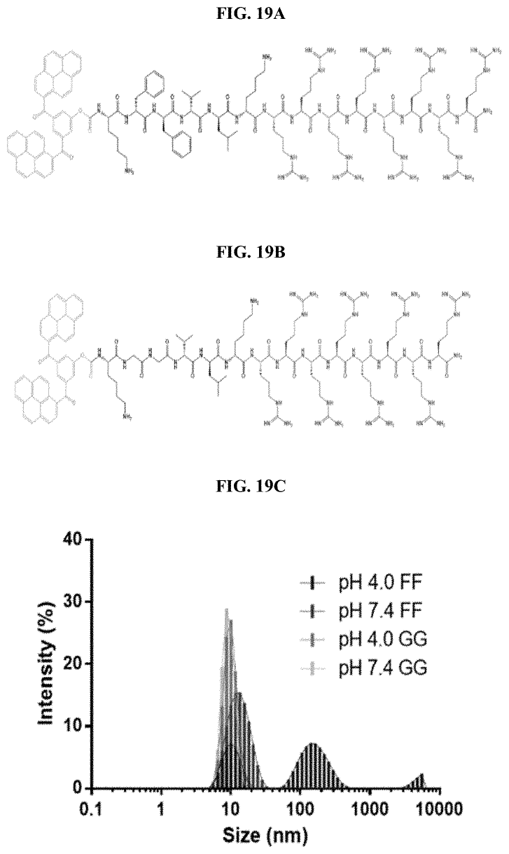

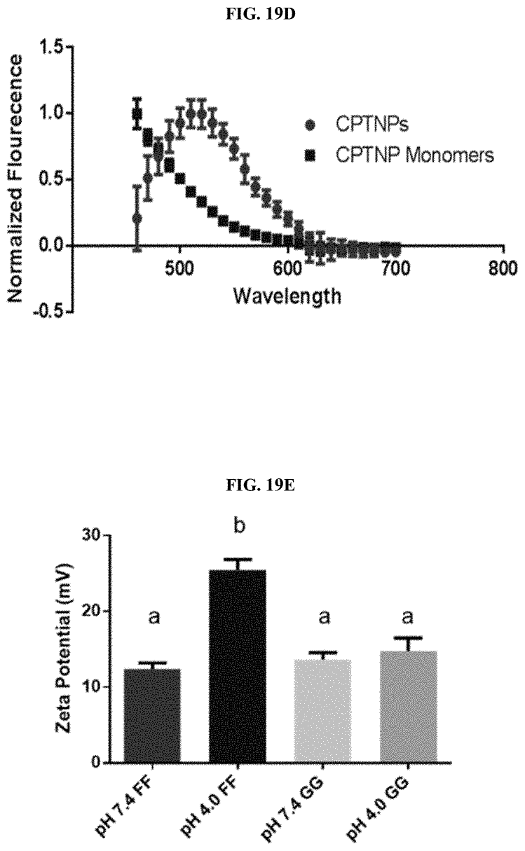

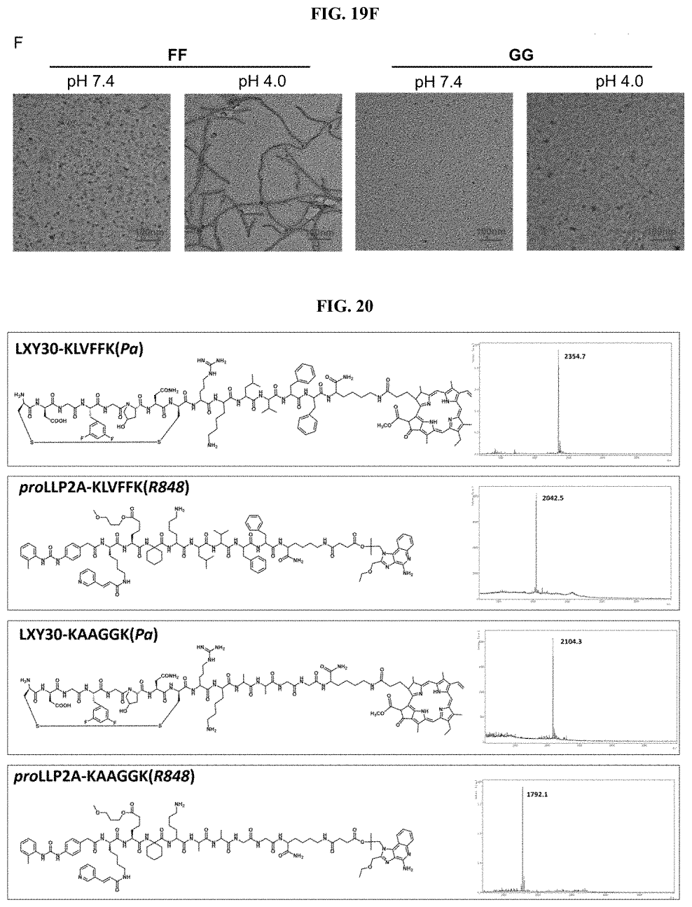

Although promising, T-NPs alone, however, was not able to completely eliminate the tumour. This may be caused by insufficient activation and homing of T effector cells in the tumour microenvironment. It is well known that tumour cells hijack PD-1 receptors of T cells by overexpression of PD-L1, which can activate PD-1, leading to inhibition of T cell proliferation, activation, cytokine production, altered metabolism and cytotoxic T lymphocytes killer functions, and eventual death of activated T cells. Clinically, antibodies targeting PD-1 or PD-L1 have been demonstrated to be able to reinvigorate the “exhausted” T cells in the tumour microenvironment. However, except for melanoma and non-small cell lung cancer, the clinical response rate of ICB anti-PD-1 or anti-PD-L1 therapy is limited and most patients are still refractory. One critical reason is that there are not enough Teff cells in the tumour microenvironment. Our receptor-mediated fibrillar transformable nanoplatform (promoting T cells homing and improving tumour microenvironment) may be able to correct such deficiency, and therefore will greatly synergize PD-1 and PD-L1 checkpoint blockade immunotherapy. Syngeneic orthotopic 4T1 breast cancer-bearing mice were randomized into four groups for anti-PD-1 antibody (anti-PD-1) therapy with or without additional nanoplatform: (1) anti-PD-1 alone; (2) regimen 4 (UT-NPs) plus anti-PD-1; (3) regimen 5 plus anti-PD-1; (4) regimen 6 (T-NPs) plus anti-PD-1. When tumour volume reached about 100 mm³, NPs were given i.v. injected on day 1, and anti-PD-1 given i.p. on day 2. The same cycle was repeated on day 3, 5, 7, and 9 for a total of 5 cycles, and mice were observed continuously for 21 days (FIG. 18A). Not unexpectedly, anti-PD-1 alone and regimen 4 plus anti-PD-1 treatment were ineffective (FIG. 18B). In contrast, regimen 5 plus anti-PD-1 treatment did significantly suppress tumour growth, resulting in a longer median survival, compared with 8 treatments of regimen 5 without anti-PD-1 as shown in FIG. 18B,18D (49.5 d vs. 39 d); both of these treatments however were not able to completely eliminate the tumours. Most remarkably, mice treated with regimen 6 (T-NPs) plus anti-PD-1 resulted in gradual shrinkage and eventual complete elimination of tumours within 21 days, and without any sign of recurrence during the observation period of 90 days (FIG. 18C), validating the synergistic effects of our transformable nano-immuno-platform T-NPs with checkpoint blockade immunotherapy.

Unlike traditional chemotherapy or targeted therapy in clinical oncology, immunotherapy can potentially induce an adaptive response with capacity for memory. Memory is crucial to achieving durable tumour responses and preventing recurrence, which often leads to mortality. To assess whether the synergistic therapy of T-NPs with immune checkpoint anti-PD-1 therapy (T-NPs plus anti-PD-1 Ab) could induce a memory response, the cured mice from previous experiment was re-challenged (FIG. 18A-18C) with 4T1 cells on the opposite mammary fat pad on day 90; naive mice of the same age were used as a negative control (FIG. 18D). In this experiment, the mice were given anti-PD-1 via i.p. three times on day 91, 93 and 95. The tumour volume of all the naïve mice increased rapidly within 30 days even with the injection of anti-PD-1 (FIG. 18E). However, either no tumour growth or significant delay in tumour growth was observed in mice previously treated successfully with T-NPs plus anti-PD-1 treatment (FIG. 18F), confirming the presence of an excellent immune memory response exerted by these previously treated mice. Survival curves of this experimental group correlated well with tumour growth results (FIG. 18G). All mice remained alive during the 60-day observation period (day 90-150). In addition, the serum levels of cytokines such as TNF-α and IFN-γ in this experimental group were found to be much higher than those in the control same age naïve mice group after re-challenged with 4T1 tumour cells for 6 days (FIG. 18H-18I). These results suggest that a durable and robust T cell memory response was generated by regimen 6 (or T-NPs) plus anti-PD-1 given previously.

In addition to 4T1 syngeneic orthotopic breast cancer model, similar therapeutic study in Lewis lung syngeneic subcutaneous murine tumour model was performed with excellent results (FIG. 18J-18L). Complete tumour regression and prolonged survival was obtained for therapy with T-NPs plus anti-PD-1. No systemic toxicity and weight loss were detected.

In spite of the clinical success of checkpoint blockade immunotherapy, only a fraction of cancer patients benefits from this therapy. Defects in Teff cells homing to the tumour sites is probably one the main reasons why many patients remain refractory to such treatment. Development of approaches to convert an immunologically “cold” tumour to a “hot” tumour is undergoing intense investigation around the world. The receptor-mediated transformable nanoparticles (T-NPs) described herein can provide a relatively simple solution to this challenge. By incorporating pro-ligand LLP2A and R848 to the nanoparticle, it has been demonstrated in syngeneic 4T1 breast cancer and Lewis lung cancer model that this non-toxic treatment can (1) facilitate the homing of T-cells to the tumour sites, (2) promote retention of T-cells at close proximity to the tumour cells, and (3) provide sustained release of R848 at the tumour microenvironment, resulting in the re-education of TAMs to M1 phenotype. Since the nanoplatform is modular, there are options of combinatorially incorporating various different ligands, pro-ligands, or immunomodulators to the nanoplatform. One unique feature of the immune-nanoplatform is that the nanofibrillar network formed at the tumour microenvironment is durable, which may explain its remarkable in vivo anti-tumour immune response and memory effects but without any sign of systemic immunotoxicity, even when given in conjunction with anti-PD-1 antibody. The pro-ligand concept of using LLP2A to capture T-cells at the tumour site is innovative, and may be applied for capturing other beneficial immune cells, including natural killer cells. Other potent immunomodulators against other pathways such as the stimulator of IFN genes (STING) pathway may also be tried. The nanoplatform is highly modular and may appear to be complicate. However, in reality, it is highly robust. Each transformable peptide monomer is chemically well-defined, and the final immune-nanoparticle can be assembled by simple mixing in DMSO followed by dilution with water. Scale-up production for clinical development should not be a problem.

Statistical analysis. Data are presented as the mean±standard deviation (SD). The comparison between groups was analyzed with the student's t-test (two-tailed). The level of significance was defined at *p<0.05, **p<0.01 and ***p<0.001. All statistical tests were two-sided.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

Sequence Listings:

SEQ ID NO:1: KLVFF

SEQ ID NO:2: klvff

SEQ ID NO:3: FFVLK

SEQ ID NO:4: YCDGFYACYMDV

What is claimed is:

1. A compound of formula (I):
A-B-C (I)
wherein
A is a hydrophobic moiety;
B is a peptide, wherein the peptide forms a beta-sheet; and
C is the hydrophilic targeting ligand, wherein the hydrophilic targeting ligand is a LLP2A prodrug, LLP2A, LXY30, LXW64, DUPA, folate, a LHRH peptide, a HER2 ligand, an EGFR ligand, or a toll-like receptor agonist CpG oligonucleotides; and
wherein when the hydrophobic moiety is bis-pyrene, then C is a LLP2A prodrug, LLP2A, LXY30, LXW64, DUPA, folate, a LHRH peptide, an EGFR ligand, or a toll-like receptor agonist CpG oligonucleotides.

2. The compound of claim 1, wherein the hydrophobic moiety is a dye or a drug.

3. The compound of claim 1 or 2, wherein the hydrophobic moiety is a chemotherapeutic agent, a fluorescent dye, an immunomodulatory agent, a toll-like receptor agonist, a small molecule agonist of stimulator of interferon gene (STING), porphyrin, cholesterol, vitamin D, or vitamin E.

4. The compound of any one of claims 1-3, wherein the hydrophobic moiety is paclitaxel, bis-pyrene, cyanine dye, resiquimod, gardiquimod, amidobenzimidazole, porphyrin, cholesterol, vitamin D, or vitamin E.

5. The compound of any one of claims 1-4, wherein the hydrophobic moiety is resiquimod or porphyrin.

6. The compound of any one of claims 3-5, where the porphyrin is pyropheophorbide-a, pheophorbide, chlorin e6, purpurin or purpurinimide.

7. The compound of any one of claims 3-6, wherein the porphyrin has the following structure:

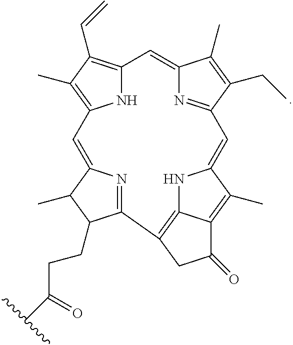 [SMILES: *C(=O)CCC1c2nc(cc3[nH]c(cc4nc(cc5[nH]c6c2CC(=O)c6c5C)C(CC)=C4C)c(C=C)c3C)C1C]

8. The compound of any one of 3-6, wherein the porphyrin is pheophorbide-a.

9. The compound of any one of claims 1-8, wherein the peptide is a peptide sequence 5-20 amino acids in length.

10. The compound of any one of claims 1-9, wherein the peptide is a peptide sequence 5-15 amino acids in length.

11. The compound of any one of claims 1-10, wherein the peptide comprises a peptide sequence from a beta-sheet peptide domain of a beta-amyloid peptide.

12. The compound of claim 11, wherein the beta-amyloid peptide is beta-amyloid 40.

13. The compound of any one of claims 1-12, wherein the peptide comprises at least 50% sequence identity to SEQ ID NO:1.

14. The compound of any one of claims 1-13, wherein the peptide comprises SEQ ID NO:1.

15. The compound of any one of claims 1-12, wherein the peptide comprises at least 50% sequence identity to SEQ ID NO:2.

16. The compound of any one of claims 1-13, wherein the peptide comprises SEQ ID NO:2.

17. The compound of any one of claims 1-12, wherein the peptide comprises at least 50% sequence identity to SEQ ID NO:3.

18. The compound of claim 17, wherein the peptide comprises at least 80% sequence identity to SEQ ID NO:3.

19. The compound of claim 17 or 18, wherein the peptide comprises SEQ ID NO:3.

20. The compound of any one of claims 1-19, wherein the hydrophilic targeting ligand is the HER2 ligand, wherein the HER2 ligand is an anti-HER2 antibody peptide mimic derived from the primary sequence of the CDR-H3 loop of the anti-HER2 rhumAb 4D5.

21. The compound of claim 20, wherein the HER2 ligand has at least 50% sequence identity to SEQ ID NO:4.

22. The compound of claim 20 or 21, wherein the HER2 ligand has at least 80% sequence identity to SEQ ID NO:4.

23. The compound of any one of claims 20 to 22, wherein the HER2 ligand is SEQ ID NO:4.

24. The compound of any one of claims 1-19, wherein the hydrophilic targeting ligand is a LLP2A prodrug, LLP2A, LXY30, DUPA, folate, a LHRH peptide, or an EGFR ligand.

25. The compound of any one of claims 1-19, or 24, wherein the hydrophilic targeting ligand is a LLP2A prodrug, LLP2A, or LXY30.

26. The compound of any one of claims 1-19, or 24-25, wherein the hydrophilic targeting ligand is a LLP2A prodrug, with the following structure:

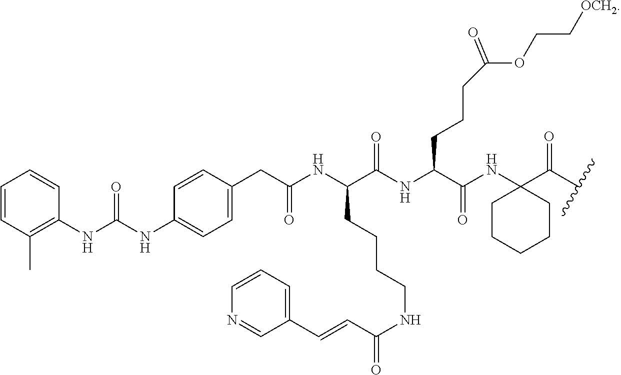

27. The compound of any one of claims 1-19, or 24-25, wherein the hydrophilic targeting ligand is LLP2A, with the following structure:

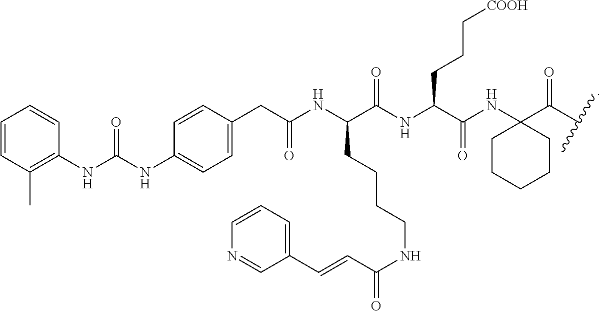 [SMILES: *C(=O)C1(NC(=O)[C@H](CCCC(=O)O)NC(=O)[C@@H](CCCCNC(=O)/C=C/c2cccnc2)NC(=O)Cc2ccc(NC(=O)Nc3ccccc3C)cc2)CCCCC1]

28. The compound of any one of claims 1-19, or 24-25, wherein the hydrophilic targeting ligand is LXY30, with the following structure:

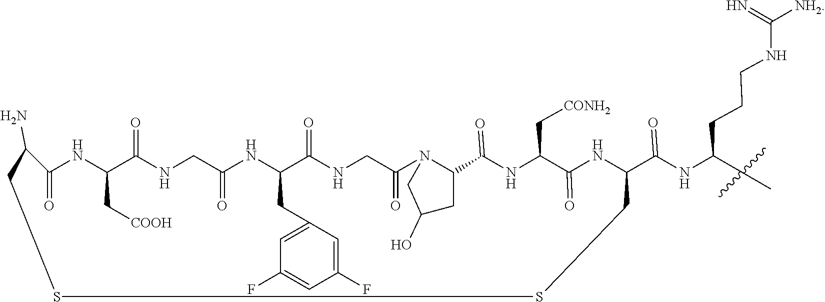

29. The compound of any one of claims 1-7, 9-14, 24-25, or 28, having the structure:

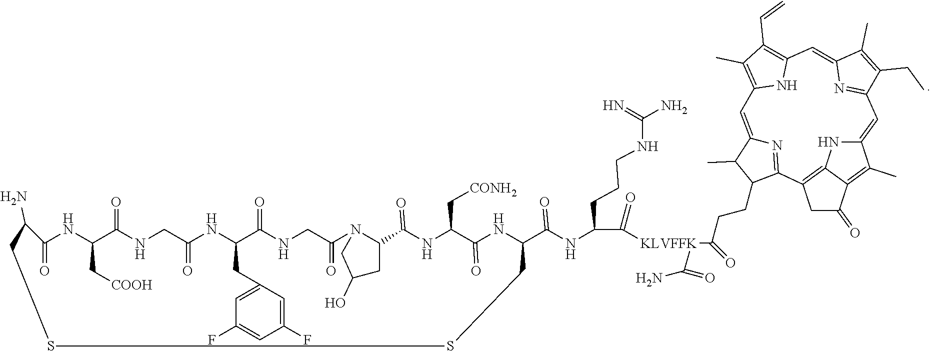

30. The compound of any one of claims 1-5, 9-14, or 24-26, having the structure:

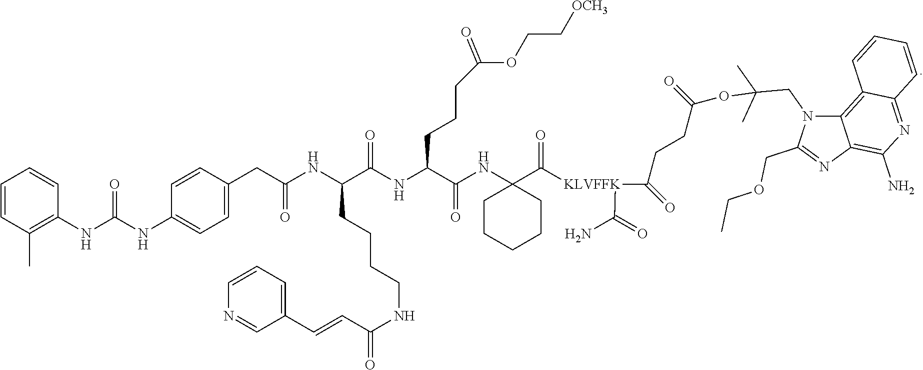

31. The compound of claim 30, wherein the compound is converted in situ to the following structure:

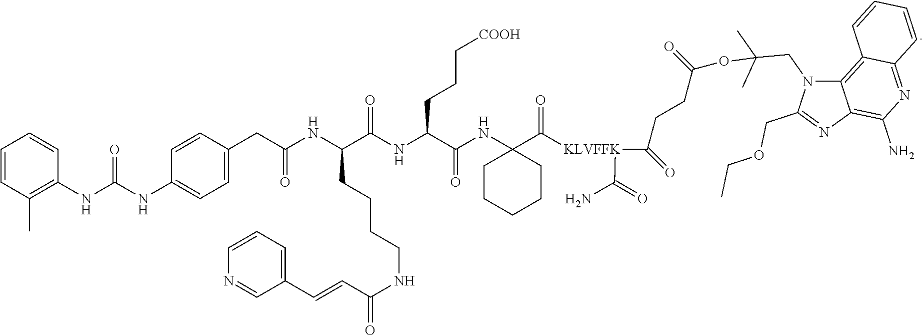

32. The compound of any one of claims 1-5, 9-14, 24-25 or 28, having the structure:

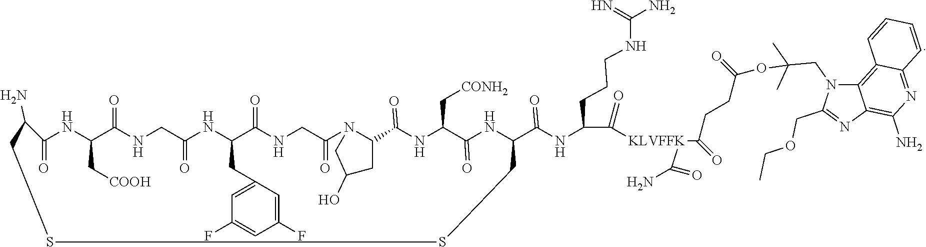

33. A nanocarrier having an interior and an exterior, the nanocarrier comprising a plurality of compounds of any one of claim 1-32, wherein each compound self-assembles in an aqueous solvent to form the nanocarrier such that a hydrophobic pocket is formed in the interior of the nanocarrier, and a hydrophilic group self-assembles on the exterior of the nanocarrier.

34. The nanocarrier of claim 33, wherein the nanocarrier further comprises a hydrophobic drug or an imaging agent sequestered in the hydrophobic pocket of the nanocarrier.

35. A nanocarrier having an interior and an exterior, the nanocarrier comprising a plurality of a first conjugate and a second conjugate wherein the first conjugate comprises formula (I):
A-B-C (I); and
the second conjugate comprises formula (II):
A′-B′-C′ (II)
wherein:
A and A′ are each independently a hydrophobic moiety;
B and B′ are each independently a peptide, wherein each peptide independently forms a beta-sheet; and
C and C′ are each independently a hydrophilic targeting ligands, wherein each hydrophilic targeting ligand is independently a LLP2A prodrug, LLP2A, LXY30, LXW64, DUPA, folate, a LHRH peptide, a HER2 ligand, an EGFR ligand, or a radiometal chelator; and
wherein A and A′ are different hydrophobic moieties and/or C and C′ are different hydrophilic targeting ligands.

36. The nanocarrier of claim 35, wherein each hydrophobic moiety is independently a dye, a drug, or a radiometal chelator.

37. The nanocarrier of claim 35 or 36, wherein each hydrophobic moiety is independently a bis-pyrene, porphyrin, resiquimod, or gardiquimod.

38. The nanocarrier of any one of claims 35-37, wherein each hydrophobic moiety is independently a porphyrin or resiquimod.

39. The nanocarrier of claim 37 or 38, wherein the porphyrin is pyropheophorbide-a, pheophorbide, chlorin e6, purpurin or purpurinimide.

40. The nanocarrier of any one of claims 37-39, wherein the porphyrin is pheophorbide-a.

41. The nanocarrier of any one of claims 37-39, wherein the porphyrin has the following structure:

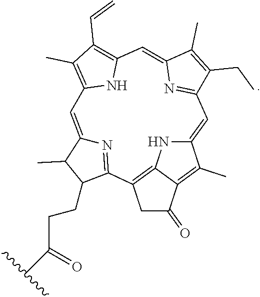 [SMILES: *C(=O)CCC1c2nc(cc3[nH]c(cc4nc(cc5[nH]c6c2CC(=O)c6c5C)C(CC)=C4C)c(C=C)c3C)C1C]

42. The nanocarrier of claim 37 or 38, wherein the resiquimod has the following structure:

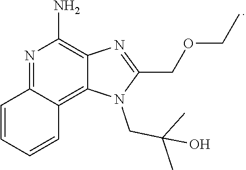 [SMILES: CCOCc1nc2c(N)nc3ccccc3c2n1CC(C)(C)O]

43. The nanocarrier of any one of claims 35-42, wherein each peptide is independently a peptide sequence 5-20 amino acids in length.

44. The nanocarrier of any one of claims 35-43, wherein each peptide independently comprises a peptide sequence from a beta-sheet peptide domain of a beta-amyloid peptide.

45. The nanocarrier of claim 44, wherein the beta-amyloid peptide is beta-amyloid 40.

46. The nanocarrier of any one of claims 35-45, wherein each peptide independently comprises at least 50% sequence identity to SEQ ID NO:1.

47. The nanocarrier of any one of claims 35-46, wherein each peptide independently comprises SEQ ID NO:1.

48. The nanocarrier of any one of claims 35-45, wherein each peptide independently comprises at least 50% sequence identity to SEQ ID NO:2.

49. The nanocarrier of any one of claims 35-46, wherein each peptide independently comprises SEQ ID NO:2.

50. The nanocarrier of any one of claims 35-49, wherein each hydrophilic targeting ligand is independently a LLP2A prodrug, LLP2A, LXY30, folate, a LHRH peptide, a HER2 ligand, an EGFR ligand, a Gd(III) chelator, a DOTA chelator, or a NOTA chelator.

51. The nanocarrier of any one of claims 35-50, wherein each hydrophilic targeting ligand is independently a LLP2A prodrug, LLP2A or LXY30.

52. The nanocarrier of any one of claims 35-51, wherein each hydrophilic targeting ligand is independently a LLP2A prodrug, with the following structure:

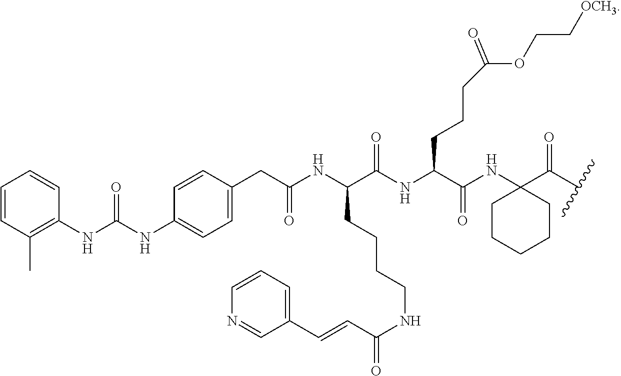 [SMILES: *C(=O)C1(NC(=O)[C@H](CCCC(=O)OCCOC)NC(=O)[C@@H](CCCCNC(=O)/C=C/c2cccnc2)NC(=O)Cc2ccc(NC(=O)Nc3ccccc3C)cc2)CCCCC1]

53. The nanocarrier of any one of claims 35-51, wherein each hydrophilic targeting ligand is independently LLP2A, with the following structure:

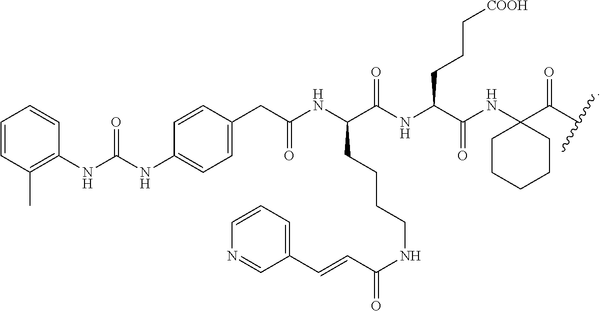 [SMILES: *C(=O)C1(NC(=O)[C@H](CCCC(=O)O)NC(=O)[C@@H](CCCCNC(=O)/C=C/c2cccnc2)NC(=O)Cc2ccc(NC(=O)Nc3ccccc3C)cc2)CCCCC1]

54. The nanocarrier of any one of claims 35-51, wherein each hydrophilic targeting ligand is independently LXY30, with the following structure:

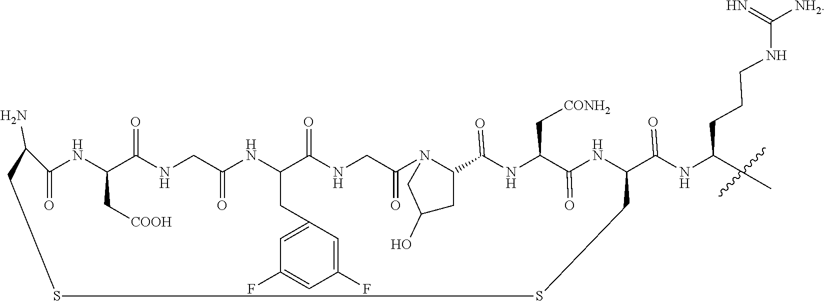

55. The nanocarrier of any one of claims 35-51, wherein the first conjugate has the structure:

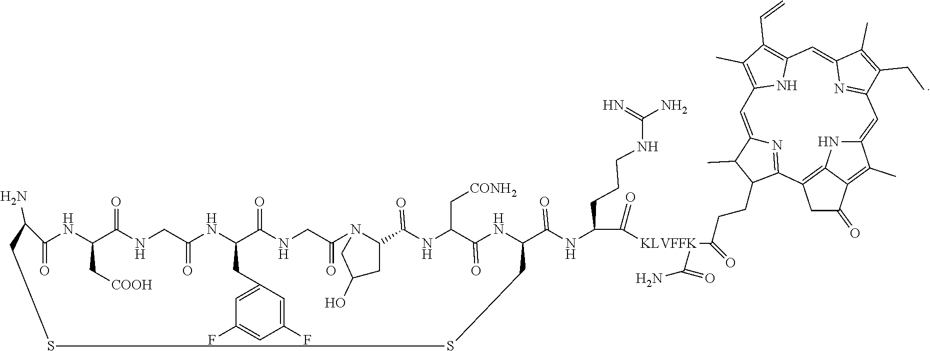

56. The nanocarrier of any one of claims 35-55, wherein the second conjugate has the structure:

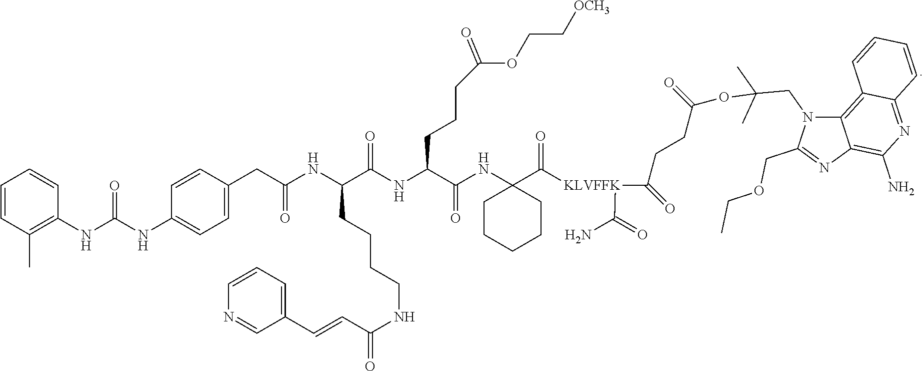

57. The nanocarrier of claim 56, wherein the second conjugate is converted in situ to the following structure:

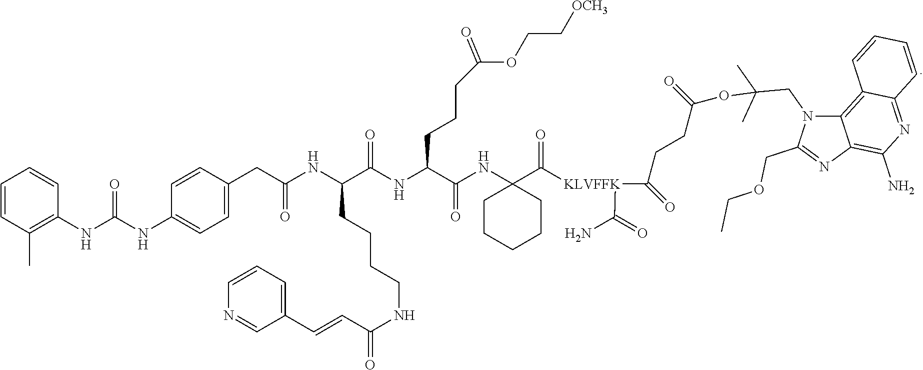

58. The nanocarrier of any one of claims 35-57, wherein the ratio of the first conjugate to the second conjugate is about 10:1 to about 1:10.

59. The nanocarrier of any one of claims 35-58, wherein the ratio of the first conjugate to the second conjugate is about 1:1.

60. A method of forming nanofibrils, comprising contacting a nanocarrier of any one of claims 33-59 with a cell surface or acellular component at a tumor microenvironment, wherein the nanocarrier undergoes in situ transformation to form fibrillary structures, thereby forming the nanofibrils.

61. A method of treating a disease, comprising administering to a subject in need thereof, a therapeutically effective amount of a nanocarrier of any one of claims 33-59, wherein the nanocarrier forms nanofibrils in situ after binding to a cell surface or acellular component at the tumor microenvironment, thereby treating the disease.

62. The method of claim 61, wherein the disease is cancer.

63. The method of claim 61, wherein the disease is selected from the group consisting of bladder cancer, brain cancer, breast cancer, cervical cancer, cholangiocarcinoma, colorectal cancer, esophageal cancer, gall bladder cancer, gastric cancer, glioblastoma, intestinal cancer, head and neck cancer, leukemia, liver cancer, lung cancer, melanoma, myeloma, ovarian cancer, pancreatic cancer and uterine cancer.

64. A method of imaging, comprising administering to a subject to be imaged, an effective amount of a nanocarrier of any one of claims 33-59.
